SEQUENCE LISTING (77 sequences)

SEQ ID NO: 1 - protein, 417 aa, Escherichia coli [sequence omitted]

SEQ ID NO: 2 - protein, 405 aa, Bifidobacterium longum
MALDVGKALKSKTLVVGVLSMSLLMSASNAVSGTIPAMKEAFSDYSAANVELLTTVPTIGSMVGTALTGLFANAIGRKKIAMAGFLISAVTGVIPAFFPYYWPILISRMFFGFGSALFVTLSVSYITDLYDGDMQRKLLGWRQAVGNLGDVVLLFVASLLITINWQSTYLIFFLLFVPMVLVGMFIPKEFDNFSIRSALVDDEGHVVDKSASQKQTTNWQVLWLAFIFLVVSMLYNVMSIKLASYVVDEGIGSASLATLIFSFLVVATILSGVLFDKVAKVTKRLTVTISEVVIGICFIATALTKNVPLMFALVLIAGFAWGIINPALTARFVDYSPAHSMNLTTSIVIIGINIGCLISPYFFALTASIFGNSSAGFAIIVGGALYLVMVVIELITLKVDKKLTV

SEQ ID NO: 3 - protein, 303 aa, Bifidobacterium longum
MTNATAQPDTSVMRKPKRQYIGILYCLPYVVVFLFGMIVPMFYALYLSFFKQSLLGGTTFAGFDNFIRAFKDEALWGGFRNVLIYAAIQIPMNLILSLVAALVLDSQRIRHIAVPRILLFLPYAVPGVIAALMWGYIYGDKYGLFGQIAGMFGVAAPNMLSKQLMLFAIANICTWCFLGYNMLIYYSALIGIPNDLYESARIDGASELRIAWSVKIPQIKSTIVMTVLFSVIGTLQLFNEPNILRTSAPDVINSSYTPNIYTYNLAFNGQNVNYAAAVSLVIGIIVMALVAVVKIIGNKWENK

SEQ ID NO: 4 - protein, 325 aa, Bifidobacterium longum
MSEAIARPRSKSLQRRDAKLALKASKHYKRMQQREPAPKLTGKQRVLNWLLHIIMAVMVIYCLVPLLWVVFSSTKTSEGIFSSFGLWFDDKNVFWQNVQDTFAYQHGVYTRWLFNTIMYAVVAGVGATIIATFAGYAIATMRFPGRNALLAVTLAFMSIPSTVITVPLFLMYSKIGLVGTPWAVIIPQLATPFGLYLMIIYAQTSIPVSLIEAAKLDGANTWTIFWKVGFPLLSPGFVTVLLFTLVGVWNNYFLPLIMLTNTNDYPLTVGLNMWLKMGAQGTSDGQVPNNLIITGSLIAVVPLIIAFMFLQKYWQSGLAAGSVKQ

SEQ ID NO: 5 - protein, 469 aa, Bifidobacterium longum
MTHKGVIMKKSIRLIAAVAALAMTAGAAACGSGTSQKNNKADVSLNDINSALTDTSKTTDLTVWAYSAKQIEGPVKAFQERYPHIKINFVNTGAASDHFTKFQNVVSANKGVPDVVQMSISEYEQYAVSGALLNFESDEIEKAWGTQYAQAAWKNVHFGGGLYGTPQDAAPLALYVRKDILDEHGLKVPTTWQEFYDEGVKLHKQDPSKYMGFISSSDTSLFGVLRTVGAKPWTVKDTTNIDFSLTTGRVAEFIKFIQKCLDDGVLRAAATGTDEFNREVNDGVYATRLEGCWQGNIYKDQNPSLKGKMVVAHPLAWGNDGESYQSESTGSMFSVSSATPKDKQAAALAFIQWVNGSKDGVSEFLTANKGNYFMASNYYQKDKSKRDQQETDGYFANTNVNEIYFESMDKVNMDWDYIPFPAQLTVAFGDTVAPALTGKGDLLTAFTKLQDNLKSYAEDNGFKVTTDAD

SEQ ID NO: 6 - protein, 739 aa, Bifidobacterium longum
MAGLFRTASKQPNPLGTAIRTRGSGKYTFGLALSAGLAGLLYGYDTVSISGAIEFLRARYGLSTLMEGLVISSIMLGAVIGAATAGFLSDRFGRKRILIIGGAFFLVAAVWSALTIGPIALIVARVAGGYGIGLTAALAVTYITESAPANIRGLLAFSYQLLAVCGLFLTNVINYIIASYGSNDWDIAIGWRWMLGLGAIPAAAFLLAMRRAPESPRFLIQVGRTDEGFAVLEHILGTERARLRTDDIQASVKLETEMSHEFHDLFRPGLRRALVIGIFLAVFNQFIGMNAISYYGPVMFSDLGFGGDTQFLAAASVGGVELVATVVGMYLIDTFGRKRLMEIGTGMMCAFALCISGSYFMGNSLLTLIFVMAFTISFAFSMGPIPWIVIPELFPTYLRGRATGLCVMCLLFANWIIAQFTPMMIDGLGGGISFAIFAFLDLICLFGIVALVPETMGRTLEEIEHLWQPKTDLASAKYSLSTADANIRHAEATLRRIENERQQALGIMDAAERARAAAQQKIFAIETAKRAEAERLTAQRAAEEAAKAAANMAAEEDTAATVSEVTTDENVPSLVRRTRGAVLVGKSSSRRHDGEYTGGPADGSVTGSAAGAMLDGAAAEAAASAAVAALADSLPDDPCTLANRPTTRSGADDTDSVDAADAVDFANDANRNRKQTNAVQPAHMASIEHAHSADTPRGVLLPSADEDSYSEHSFAETDDDREAQAIDAALDSLDALIRG

SEQ ID NO: 7 - protein, 441 aa, Bifidobacterium longum
MASATKSAWKNPSYLQSSFGIFMFFCSWGIWWSFFSRWLTDPTHGLGMTSAEQGQIYSINSLATLVIMFAYGAIQDQLGIKRKLVIFVSAIAALVGPFVQFVYAPMLTAGGTTRFIGVLIGSIVLSSGFMAGCSLFEALTERYSRKFGFEYGQSRAWGSFGYAIVALCAGFLFNINPLLNFWVGSICGLGMLCVYAFWVPAKQKEELKKEADPNAAPTNPSFKEMISVLKMPTLWVLIVFMLFTNTFYTVFDQQMFPNYYASLFPTTEIGNATYGTLNSFQVFLESAMMGVVPIIMKKIGVRNSLLLGATVMFLRIGLCGVFHDPVSVSIVKLFHSIEVPLFCLPAFRYFTLHFDTKLSATLYMVGFQIASQIGQVIFSTPMGALHDAMGDRPTFFTISGIVLAALIYGFFVIKKDDQEVGGDPFYTDKQLKAQAAAKANA

SEQ ID NO: 8 - protein, 445 aa, Bifidobacterium longum
MTHFSTVKRVIALAGAGAMLFSVAACGGVTASDGGKTTLKFAAFEGGYGADMYKEVVAAYEKLNPDVKIELTTSKKIEDEITPGMKAGNYPDIVELGQGSTSGLTETLLKDKAIEDVTDVLDMKVPGENKTVKDKLVDGVIGLYTNPYGGDKTYLMPMYYSPSGLVYNKTLLEQNGWKMPTTWDEMFKLGDEAKAKGISLFTYPTAGYFDAFFNALLADIGGDQFYQDVMTYKKDVWKTDEAKEALETTYKLVTEYLNPDTVGYANAQDFTKNQQSILDGKSLFMPNGTWIVNEMKDAPRTSGFEWGFAPVPTVKNGGTRYINTTIEAVWIPAKAKNKEAAKKFMAYLYSDEAASIFAKTGAIQPIKGNTDDLASDMKVFYDAYNEDNVKAVAGSFSATATVEGKNIKDDLYNAVDSVASGKTTLQQWQDKLNETSNALNAAAKQ

SEQ ID NO: 9 - protein, 303 aa, Bifidobacterium longum
MSSTKHTTDLGRMRSRFGILCSLPTAILLIIFIFIPTVNVFRMSVYKWSGYSDEQTFVGLSNFRKLFVDPNFIHAFQNTVLILVLVTSVTMPLAIIFAAVLTQNRIIGGSLIRFILYVPSVLSIVVVAAIFSAIYDQNNGLINGTLSFLHLNGLKQVWLGNQDIVIFSIAFAMLWQSCGYYMVMYMSSMAGIDPSLYEAAQLDGASKTRQLFSITIPLIWANIRTTLTFFIISSVNLSFVLVTALTNGGPDNASQVLLGYMYDQAYSNSSYGYGMAIGVVIFLFSFIMSLIVNRATKREVLQY

SEQ ID NO: 10 - protein, 310 aa, Bifidobacterium longum
MAYSSVSGRPAHSRRVRPAADRGSAHSWTAGRMVGQVVLVIAALGIAIPTLWVFVASFKDKSEFYGSPWTWPKGLHWQNYADAFVDAKLGQYFGTSVLVTVMSMAMVLVIALPCSYVLARFDFPGKKILKLAVQAGLFINVNYIVVPIFLMLVDADKLLYNIMPNGFFVNNVFILALVYASTSLPFTIFLLQDFFASIPKDYEEAAMIDGASQTRIMTRIFFPMAMPAISMSMLFNFLTYWNDYIISMTLMTGNNRTVQVGLLNLMQTQKAATNYGRLYAGMVIVMVPVLIFYAVVQKKLLETSTMGGIK

SEQ ID NO: 11 - protein, 59 aa, Bifidobacterium longum
MSDRTKSVIATTVKVTLYVCFVVLTIVGVGRDGWDGLCLELIGLAGVIGVVWWYNAHNR

SEQ ID NO: 12 - protein, 408 aa, Bifidobacterium longum
MAILLLAVIYVAFISLGLPDSLLGSAWPTMSQNLNVPVAWAGGISAVISMFTIVSALLSDRMTLKFGAGKVTAVSVALTAAALAGFSIAPNYWVLLAIAIPYGLGAGGVDAALNNYVAIHYESRHMSWLHCMWGVGASVGPYIMGYALSQGQGWPWGYRYIAILQVMLTVILVLSLPLWKKRGTTGGGESTDGAASSDGNAEGCGNAEGASVAERKPLGVAGVLAIRGAKEILVMFFCYCAIESTAGLWASSYMVMHSGIDKITAASWAGLFYVGITVGRALSGFLSMRFKDPVMIRLGQVLVLAGILTMFVPLPHHLGVVVGLVVIGFGCAPIYPCVIHSTPAYFGEDKSQAVVGVQMACAYVGSLLMPPLFGIIAQYVTISLYPWYLLVLLVLMVAMHERLRKLRG

SEQ ID NO: 13 - protein, 316 aa, Bifidobacterium longum
MSSATAEERARRKAERKLEKQRQAAQYDLPRSMRPSVATKTITIIVLVLVLIYFLFPIYWAIIASTKTPAQMTGSNGMWFAVPLDQLPDAIATNYSKLLGWTRGNFWRWVLNSLIYSGVSALVGTIVSVMAGYATAKFNFRGKNAAIGIIMACMLMPSALLTIPMYSIFHTLHLTNTMLSIIIPCCVSPFGFFLGRTYAQSSVPDELLEAARIDGASEARIFFTIVLRLLAPAMVTIFLFLFVATWNNFLLPLMMVSSDTLKPVTLGLYGMVSLATFTDRGALMMGALLGVLPVIVLFLGLQRYWQSGLAAGAVKG

SEQ ID NO: 14 - protein, 323 aa, Bifidobacterium longum
MTASTTHAKGKAPRRAAAKPKRSGAQKRENRTGWAFMAPFAVLFAFVFILPIIWAIYSSFFRQVTEGGGAYGGGELVNKFVGFQNFQYVITSGNFWSGVGRVLVYTLIQVPIMIIAALALAIVIDSFVVKHVTGFRLGYFLPYAIPGVVASIIWVYLYNGQISPIVKGLAAIGINVDFFSSNIVLGSMANITTWTFTGYNMLIFLAALQAIPHDLYEAARIDGANGWQIATKIKLPNVRGAALLAMLLSIVGTIQLFNEPQIMSTADPGISKAYTPMMMAMNTSQGTITPSGDGPASAIAIVMALIAGVLAVLYTLAERKVNE

SEQ ID NO: 15 - protein, 438 aa, Bifidobacterium longum
MVSHNKRIVAAFAAVAAMGMGLAGCGGSDTAGDTKSGSDGGVVNITYMHRLPDSKGMTLVNDIVAKWNKEHPNIQVKATKFDGNASEMIKKLETDVKAGNAPDLAQVGYAEVPEVFTKGLLQDVTDEAAKYKDDFASAPFALMQVDGKTYGLPQDTGPLAYFYNAAEFEKLGITVPKTADELIETAKKTAAQGKYIMTFQPDEAMMMMSGQAGASGPWYKVDGNSWVVNTQTKGSKAVADVYQQLIDNKAALTNPRWDASFDNSIQSGQLIGTVAAAWEAPLFIDSAGGTGAGEWKVTQLGDWFGNGTKTGSDGGSGVAVLKGSKHPAEAMEFLDWFNTQVDDLVSQGLVVAATTADAKTPQKWSDYFSGQDVMAEFKTANDNMGDFTYMPGFSAVGAAMKQTAAKAADGSAKVSDVFDTAQKTSVDTLKNLGLSVKE

SEQ ID NO: 16 - protein, 460 aa, Bifidobacterium longum
MKKSIRLVAAIAALAMTAGISACGSSTNGNQAKSDVTAQDVENALTDTSKNVELTVWAYSAKQMEPTVKAFEKKYPHIKINFVNTGAAEDHFTKFQNVVQAQKDIPDVVQMSANKFQQFAVSGALLNFANDSIEKAWSKLYTKTAWAQVHYAGGLYGAPQDATPLANYVRKDILDEHNLQVPESWEDIYNEGIKLHKEDSNKYMGILGSDISFFTNLYRSVGARLWKVNSVDDVELTMNSGKAKEFTEFLQKCLKDGVLEGGTVFTDEFNRSINDGRYATFINENWMGNTYKEQNPSLKGKMVVAAPPSWKGQPYQSSSVGSMMSVSAACPKEKQAAALAFINWLDSDKDAIQSWQDTNNGNFFMAASVYQDDENQRNKKETDGYYANDDVNAVYFDSMDKVNTDWEYLPFMSQVEVVFNDVIVPEMNENGDLVGAMAKAQQKLKAYAEDNGFKVTTDAD

SEQ ID NO: 17 - protein, 325 aa, Bifidobacterium longum; identical to SEQ ID NO: 4

SEQ ID NO: 18 - protein, 303 aa, Bifidobacterium longum; identical to SEQ ID NO: 3

SEQ ID NO: 19 - protein, 427 aa, Bifidobacterium longum
MASNTPTTTSKSTRGKSWIIAFALVTLLFFTWGLTMNLVSAIRDPFAVYLNLNSTESSLLQVAYFGAYFVMAIPATMVAKRFGYKGGMICGLIFFVIGSFLTIPATTSLSYGLFLLAMFVIALGAASLEANCNPYIIKLGDEKGESFRLNLAQSFNAVGSVVSPLILGMILGNTASAKPGDPGFEESKVAFMSQTRMFYIVIGVILAIVIAVFVIAKLPSPPGDEEEAAGGVAQDSSIGALVKKPWVVLGVIAEFVYVGLQTIGFSTFSTFAQQQWEGMSAGMAATFLAVLTLMYTIGRFVTTPFMAKFEPGKILGFYMIGATILMILTAMGLGKISVITFIALYFFMSIAYGTIFALALTHLKGAAAKTGSSLITMSIVGGAIIPLVYGAITDAAGMNVMLWATVPLFLFLVWYAFWGSKIGLKEA

SEQ ID NO: 20 - protein, 219 aa, Bifidobacterium longum
MKRLAVGYEFICRIIMMVAVVNVAFVLHTLLGFVLGGLFPSVAASYSVFRQWLLDVEDRSWTIKRTWTTFHQAWLEEIGAANQLGWPQTIIWLLLIWEYWFVQNNDLGYVGTAVSGVLLVLNVLYGLFVFLSWSVHINFNENLLWTVRTSLVMVVARPLCSFIVLALFLATVWSYYTWPGLMMAFGPAVPIFFTMVAVYSWGKMPGMDVRVIEPLEKKR

SEQ ID NO: 21 - protein, 320 aa, Bifidobacterium longum
MTSTSINPAPVRGAGSDDIPFNPKLRQRRSVSDWVVDIVIWLLIALVVVAIVYPIWFIVIASVSDQTMVSQGKVFLLPAKMNFGGYAKVFTDSRIWVGYRNTIFYSVAGTALNMLVTIPAAFALSRREFKPRRVILFLMTFTMFFSGGMIPSFLLYKQLGLLNSVWVFILPGAVSVWNLIVARSFFESSIPESLHDAAQIDGLGYFGYFLRIVLPLSSAILAVMTLYYFVGHWNDFFTGLVYIRDADKLPLQNVLRSILLSNQTNITGQSSGGMDVVQQRDFANQIKYGVIIVSTLPLLVLYPFLQKYFNKGVMIGAVKG

SEQ ID NO: 22 - protein, 317 aa, Bifidobacterium longum
MSVVKQGEVVDNALAARSAPLPVRLGRHFRRYGALWLMTLPALVFVGLFAYVPMYGLRLAFYDFDPVEGLMGGRFAGLRYFEQFFRSGMFVNIMVNTLRISLWTLVMGFIAPIVLALLINQISSSKIKGFVQTVTYMPHFISTVVIVSMINIFLSPSTGMIGRLFPGTDLLGEPGLFTPIYWISEVWQHMGWNCIIYLAALSSVDLSLYEAAKIDGAGRLQLIRYVDIPAIMPTVGIMLIMNMGSVLNVGFEKVLLMQNPMNLSVSEVIATYTYRMGILGNQFSYTTAIGLFNTVVNFFFLVLANFISKRASDTSIF

SEQ ID NO: 23 - protein, 530 aa, Bifidobacterium longum
MRRTAMRVGSAVCAVALLGSLAACGGDKKPTTTADGKPIVSVLVAKEPNQDKIANMQWAKDLEADCDCKIEWQEVDNGDFANQRNTTLSAGDIPDISLHAFSFVYAEQFSNLFEDLNQDLDKLPNVKAFFKEKPDAQKLNTDSKGHLYTLPNAHGKNNAGTGQLMMINKAWLDKLGLQVPTTWDELENVLKAFKTQDPNGNGQADEIPMNIKKLDPLYYTWHSPMLLLNSTGIPTGFNKGVSTYGFYAKNGVVKSFMTSDEYKQVIKYYHKLISEGLIPADWATKDDDAYNADQTSDGKTAKTGVVFSSSISDTFGDLKDQYITIPVPSAPGVSPDQTVWDGSSSEFDAAGPAVSARAANKDATLKLLNLMYSEKYSVQQFYGSFGKTVTKTGEHEYTVDNDKLIEMRKKNTSPSLSYPAGWIPDEVTIKGDAGSDALYQASKVYEKQRSNFDPVKDYIPDYVNPDADDNTTLASNYNQISNVAMQKTATWMSKGGIDEEWDAYCKQLDSLGLQENVKIWQKWYDIYTKK

SEQ ID NO: 24 - protein, 320 aa, Bifidobacterium longum; identical to SEQ ID NO: 21

SEQ ID NO: 25 - protein, 317 aa, Bifidobacterium longum; identical to SEQ ID NO: 22

SEQ ID NO: 26 - protein, 531 aa, Bifidobacterium longum
MRRRTALKAGAITCAVALLGSLAACGGSKEPTTTADGKPIVSVLVVKRPATDKIANMQWAKDLEADCDCKIEWQEVSEDAWAQQKNATLAAGKIADVSLHAFFPANAAQFPGLFEDLSKDLDKMPNVKQFFKEKPDAQKLTTDPEGHMYALPSSRGKSYSGTGQHMFINKTWLDKLGLQVPTTWDELENVLKAFKTEDPNGNGQADEIPMNIRKLDSYFTYYSPMLLLNSTGIVTGFNKGASPTGFYAKNGVVKSFLTSDEYKQVIKYYHKLISEGLIPADWATKTFDACDTDQLSDGKTAKTGVSFGWSQDASFGTLKDQYIPIPVPSAPGVSPDKTVWDGSSAEFEADRFSLSSHAANKDAALKLANLLYSEKYSVQQFLGSFGNLVTDDGNRHYTVDEDKYTKAMGDNLFPGLADRFSGWIPDGVTIKGDVDGDNLLEANKPYEEQRSHFDPVKDYIPDYVNPDPTDSNTLTNNNAQISNVVMQKTATWMSKGGIDEEWDAYCKQLDSLGLQENVKIWQKWYDIYTKK

SEQ ID NO: 27 - protein, 480 aa, Bifidobacterium longum
MSQQTQSQIDGKLKGKLIAVCTIGAVGGFLFGYDTSVINGAVTAIASPDSGFALSSTMSGFSVSSALLGCVFGAWFAGVVADRMGRIRVLELAAILFVAASLVTGLTTNFTIFIIFRIVGGLGVGFTSAIGPAYISEISPAARRGFFTSFQQMAIALGIIASLVVNDIYVVASGGAEMPFWMGIDAWRWMLVTTALPGVIMFFLCFALPETPRYLVMKGEIAKAKTILIDVIGDNNADVTIAKIQKSFQSEENEKKVSFRELRGKTFGLKGVVWISIIFVLLAQANGQNIIMLYDSSLWNMLGFSEQASLTIAVVRATLAAVATVVGMVLIDRVGRRNLMKYGTLLMTVFLGITAIGFGQGQLSADGTLSLSITWTWISIVCTYAFFLVCCATTNVTVWVIISEIFPNRIRAKGEAVATAANWMFNFLITTIYPPMRDGIGLGWTYGFYTLMALLGFFFVVKFLPETNGVELEDMKAELK

SEQ ID NO: 28 - protein, 308 aa, Bifidobacterium breve
MASADVAAVPMHTDKARENKHKADWRGWKFMWPFTVVFVFVFIIPIVYAVYISFFKKQMIGGTRFVGVENYARLLQDGQFWSSVGRVALFTCVQVPIMLFLSAAMALALDSMKLHGAKFFRISTFLPYAVPAVVSTLVWGFVYGAKYGLVGSFNDFFGTNVDVLQPGVLLASIGNISTWEFTGYNMLIFYSSLSTIPHSLYEAASIDGASEWQIVKSIKLPELKGSLAITVIFSIIGSFQLFNEPSILQNMVPGNAITTYYTPNMYAYNLSFSGNQSNYAAALAIVMAVITMAIAYAVQLKSMKEQMK

SEQ ID NO: 29 - protein, 332 aa, Bifidobacterium breve
MSAAASTRLTDAELKAREKAARKAEKERQKRIAADEAAERKRSARAGFANVNNPRRSTLMTVLCAIFAVYCLFPFVYLVINATKTQADFTSTFGLGFGKSFALWDNIVTVFTYQGGIFGRWLVNTLLYVVVGAGGATLLAIMGGYALAKFRFPGRKAVFAVIIGSISVPGIALAVPQFLLFAKLGLTNTPWAMIIPSLISPFGLYLMWIFSEQAVPTELLEAARVDGASEFRTFWTISLPLLAPGIVTTALFTIVATWNNYFLPLIMLKDADWYPLTIGLNQWKDQASTAGGQAIQNLVITGSLITIIPLVIAFLCLQKYWQSGLAAGAVKE

SEQ ID NO: 30 - protein, 691 aa, Bifidobacterium breve
MEHREFKWPQPLAGGKPRIWYGGDYNPDQWPEEVWDEDVALMQKAGVNLVSVAIFSWAKLEPEEGVYDFDWLDRVIDKLGKAGIAVDLASGTASPPMWMTQAHPEILWVDYRGDVCQPGARQHWRATSPVFLDYALNLCRKMAEHYKDNPYVVSWHVSNEYGCHNRFDYSEDAERAFQKWCEKKYGTIDAVNDAWGTAFWAQRMNNFSEIIPPRFIGDGNFMNPGKLLDWKRFSSDALLDFYKAERDALLEIAPKPQTTNFMVSAGGAGIDYDKWGYDVDFVSNDHYFTPGEAHFDELAYSASLCDGIARKNPWFLMEHSSSAVNWRPINYRVEPGELVRDSLAHLAMGADAICYFQWRQSKAGAEKWHSSMVPHAGPDSQIFRDVCELGADLNKLADEGLLSTKLVKSKVAVVFDYESQWATEHTATPTQEVRHWTEPLAWFRALADNGLTADVVPVRGPWDEYEAVVLPSLTILSEETTRRVREYVANGGKLFVTYYTGLVDDKDHVWLGGYPGSIRDVVGVRVEEFAPMGNDFPGAMDHLDLDNGTVAHDFADVITSVADTAHVVASFKADKWTGFDGAPAITINDFGDGKAAYVGARLGREGLAKSLPALLEELGIETSAEDDRGEVLRVERADETGENHFVFLFNRTHDVAIVDVDGEPLVASLAQVNESEHTAAIQPNGVLVVKL

SEQ ID NO: 31 - protein, 441 aa, Bifidobacterium breve
MNNTTRAVIATAAIGALLIPLAGCGNTTTSSGKTELTVWSWDASIDRAAKSFMKANPDITVKVSNVGSTEETYTALNNAAQAGSGLPDVTLIEYLAIPQFVHSDTLMDLSKVYNTAKLKDTFTPGTWNSVNINGGLYAMPADSGPMAYFYDKDVFDKAGISEPPTTWDEFYEDAKKIRATGSYITSDSGDAGLFNAMMWAMGGHAYKLNDDKLTINIAKDKGAQRFMDLWQKMRDEDLIDVHTKTWTDDWMKSLGDGSIASLISGAWMANNLLQGVPQATGKFRVALLPTIDGTPINGEYGGSGLAITKKIPDGKLDAAKKFVEYVTTNDEGIDARVSNGSFPATTKTLNQKDFLAKTTLRNTDGSDNEFFGGQKYNEVFSQAAKDVTGKWEFLPFEPYARSVYNDNMGSFFAGKTSFRTAADNWQKALKDYAKDQGFTVQ

SEQ ID NO: 32 - protein, 455 aa, Bifidobacterium breve
MTFVARSVRYVLRKRVRTIVLLIVLTIITASMLSTIAVSRAAQQAAGRIEKEAVGGFVLASNLQGSMLTPRGGGMVRPADVRRIAQLPGVDSYMVRQNATADLVGANVAKVPGGDDYDATKEQQFGNAANVIGTNDSSELNVFTSHTLGMAEGRHLKASDKYTSMIHEDLAKANGLKVGDTLTLKANAYDADNESHSTATVKTTIVGIFKGDSARKVSSRAELTANTIYTDLDTTRDLYQYKDGKEIYQDATFVLSKGVDVEKTMDAAKKLPVDWNNYQITRNDQYSSSMLHAARGVRSMMRGALIGVTVSAVLVLSLMLLLWMNDRRQEMGILVSLGVGKPSLVAQYLAEMVLIGLPSLALGWLCAQGMAQWLGTTALHSVNASAAKELSSMGQVGGDLESSMSVRTLDSLTVSVDGTAMLYVALGLLAVMLVCVAVACMPMLRKSPRDLSEIR

SEQ ID NO: 33 - protein, 407 aa, Bifidobacterium breve
MMSMNVWKRATLAIVRKPVRSGIIGLLMLMVFTSLVAQVGVSTALRTMSDSIGAGMGIGFTVSAGENPISAEEASRFSRIPGVTKTAYATKTLAQVDGARPVMPRQGPRLDSDLAMQVSVLGTTDSSLSEEFQSGLYRLEQGRHISGDGDNVLVHRDFALQNGLSVGSTFRLRQEGRNATVRVAGIFSGNVQAQSPLPSDTSENLIYSGRRVTSALTGNDQIGMIRCLSDNPQDLSAAVSQAKTMAGGKYDVTDDSARLSGVLQSVQTVRDLVRMVLLSVCLADVLVLAMALVFWIRSRIHEIGTLLALGIGKMRIVAQLAIETGLMAAVAALCSLGTGAMLSGYVSSRLLRDSGVAPLESLHVEALPPEQTMLILLLGCAVIAVALAVSCAAVLSKSPKSILSSMR

SEQ ID NO: 34 - protein, 211 aa, Bifidobacterium breve
MSNCLQLDHVDYAYGAAKILVLSDVSADFESGKMYAITGPSGAGKSTLLSLLAGLDAPSRGVVRFEGEDIAASGYAKHRREHVSLVFQDHNLIDYLTPEENLRLVSTKADMKILEELGLSREESKRNIMHLSGGQRQRVAVGRALVAPGRAILADEPTGSLDPEMTDEVIHLLQHAAHQIGKCVIVVTHSKRVADSADVVLRLRSKKLARA

SEQ ID NO: 35 - protein, 454 aa, Roseburia hominis
MRKKLVGTLLIAAMTASTLAGCGSSNSTAGTSASGGAAETSPAESATEAEGTDAADGEEVLTVWCWDPTFNVYAMKQAEAIYQKDHPNFKLDIQEKVYTDIEQNIITAAEADNYDTLPDIFLMQDNSFQKNIANYPEVFTELSDSGINFDDFSGGKLGDSTAEGKHYGIPFDNGASIMAIRSDMVEAAGLTVEDFKDTTWSEFMELAKKVVDANGVPMLTSSGGSEIVIEMLQSAGASPIVNGEVHLADNAALKKAIEVYKQLIDEGIMVDYTDWDQYIASMNKGEAAGVIQGCWIMSSIQSAEDQSGKWAIVNMPALDGIDGATNYANCGGASWAVSSNCKNTELAFDFLKTTFGGSVELYDDLLPNAGAISSYLPAAQSDVYNQTSDFYGGQAVYKDIVEFAGKVPAINYGAYYNDIRSALTDAITNVVQNGADIDEEIQNAQDAVEFNISE

SEQ ID NO: 36 - protein, 292 aa, Roseburia hominis
MKQKKAKMSLEKKHNLTGWAFLIPAALLIFIFCFYPMIQAIILSFTKKGSGFAGLANYQRILKDKTFQQCLFNTVFYFIIQVPVMLLLALGLAQLLNSPKIKGKSIFRTLIFLPCATSLVSYSMIFKSLFAQDGLVNNIITTLGFDRVDWFQNAWAARFVIIIALIWRWTGYNMVFYLAGLQNIDDSVYEASYIDGATPFQQFMKITIPLLRPTILLTAIMSTSGTLQLFDESVNLTSGGPGKSTMTLAHYVYNISFVETPKFNYAAAISVFILVSVAVLSAIQMKVGDKRD

SEQ ID NO: 37 - protein, 268 aa, Roseburia hominis
MTKVKTTIAYIILIIASFLSAFPLYYMICGATNSSIDIVRGKLFPGVYLMENYQSLIANQNLGRAMFNSFRNAIVITVIALLICSIAGYGFEIYHDKGKDLLMNILLLAMMLPFVAIMIPLFKMFSSWKLVNTWIALALPSLSTPFLIMMFRQAARSFPHDIIQAARLEGLSEIRIFFTMFLPIMKSTYGAAMTVTFMNAWNNYLWPTIILQDSNQITMPMLVANLKSGYSVDYGMLMLGVLICTLPTAIIFLCLQKSFANGIAGAVK

SEQ ID NO: 38 - protein, 446 aa, Eubacterium ramulus
MKKKVISLMLIGAMVASMAACGSNGGDKGGTKDNDTSAAAESDGGASEGGDTLTVWTWDPKFNIPAIKEAANIYKADHPDFNLKVVETLSDDCESKLIAAAEGGDLSTLPDIVLMQDNSYQKFVTAYPEAFEDLTDSGIDFSQFPAGKLAYSTVDGKNYGVPFDNGAVIGAYRTDILEQAGYTTADLTDIDWNRFIEIGKDVKAKTGTYMLSGQADSPDTIMMMLQSAGASLFDEDGKPAMTDNAALKECIDIYKTMVDEGIYLEVNKWDDYVTSITKGSVAGVINGNWIIATIQGMEDTAGKWEITNMPKLIKTPNATNYSNNGGSSWYITSNCKNKDLAIDFLKSTFAGSVELYDKILASTGAIATYLPAGESEKYAEPVAYWSDQPIYKTIVDYSKLTPANNTSPYYYDGRNAIGTQIQNIMNGADTDSAIADAQAEVEFSMQ

SEQ ID NO: 39 - protein, 297 aa, Eubacterium ramulus
MEPKKKGMTLVAKQKAAGWLFLVPASAMIAIMSFFPMVKAFIISLQTGRGLNMHFAGFSNYVRMFQDKVFLRSVGNTFFYLIIQVPIMLVLAILLAQLLNNRDLKLRGFFRTCVFLPCATSLVSYALIFRFLFAQDGFINNALIGLGILDTGYNFLGNAMSAKFVIIIALVWRWTGYNMVFYLAGLQNIEYSVYEAAKIDGANGWKTFWSITVPLLKPTIIMTFIMSINGTLQLFDESVNLTNGGPANATITMSHYIYNSCFVRSPNFGYASAMSFFVFILVAILAFINMKVGDTRD

SEQ ID NO: 40 - protein, 276 aa, Eubacterium ramulus
MTKKKNKSMIQRRLIPTYIFLAIVSLVSVFPFYWMIMAATNDYVDISRGKLTFGTAALENFRNLIDQQPLALAMKNSFVYAISVTVICLLICSLAGYGYEIYHDKWKDKLFSVILLGMMIPQVATIVPLYRMFTKAHLINTAVGFFLPMISTPFMIMMFRQNSRAFPVDIVEAARIDGLSEIKIFFQMYMPVMKSTYAAAAVITFMNAWNAYLWPKVIMTDGDSLNMVMLIANMTGGYKLDYGMIMMGVLFCSIPTMVVFFVLQKQFAEGITGAVK

SEQ ID NO: 41 - protein, 291 aa, Bifidobacterium catenulatum
MRKPKREYIGILYCLPYVVVFLFGMIIPMFYALYLSFFKQSLLGGTTFAGFDNFLAAFKDEALWSGFRRVLIYAAIQIPLNLILSLAAALILDSQRIRHIAVPRILLFLPYAVPGVIAALMWGYIYGDKYGLVGQIASMIGATAPNMLSEKLMLFAIANICTWCSLGYNMLIYYSALIGIPNELYESARIDGASELRIAWSVKIPQIKGSIMMTVLFSVIGTLQLFNEPSILMNSAPNVITSNYTPNLYAYNLAFNGQNLNYAAAVSLVIGVIVMGLVAVVKVIGNKWENK

SEQ ID NO: 42 - protein, 325 aa, Bifidobacterium catenulatum
MSEASAKPRSKSLQRRDAKLALKASQRYKRMQQREAAPKLTGKQRVLNWLLHIIMAMMVIYCLVPLLWVFFSATKTNEGMFSSFGLWFDDKNVFWQNVQETLAYQHGTYARWLLNTVMYAVVAGFGATIIATLAGYAIATMRFPGRNSLLAITLAFMSIPATVISVPLFLMYSKIGLVGTPWAVIIPQLATPFGLYLMIIYAQTSVPTSLLEAAKLDGANSWTIFWKVAFPLLSPGFVTVLLFTLVGVWNNYFLPLIMLSDTNDYPMTVGLNMWLKMGAEGTADGQIPNNYIVTGCLIAVAPLIIAFMFLQKYWQSGLSAGAVKQ

SEQ ID NO: 43 - protein, 463 aa, Bifidobacterium catenulatum
MKKMTRIVSALAALTMVASVAACGSSTGAKDTKTAVTVEDINSALTDTSKKVELNVWGFSGKQMAGPIEAFQKKYPHIKIKFTNTGAASDHFTKFQNVVAANKDVPDVVQMSADRFEQYAISGALLNFANDDIEKAWSKLYAKTAWSQVHYAGGLWGVPTDATPLAMYVRKDILDEHNLKVPTTWQEFYEEGVKLHKEDPSKYMGVLINNDISCFTDLLRSAGARPWKVNSVDDISLSMTSGTTKEFIEFLQKCLQDGVMEAAPGWTDAFNRATNEGTYATYISQNWQGNSYKGQNPSLKGLIQVTLPPAFGDSADDFKSSSVGSMMSVSAACPKDKQAAALAFINWLDSDKEAIQSWQDNNHGNYFMAASAYQDSEDIRNMKETDGYFVNTDVNSVYFESMDKVNTDWEYLPFMSQVGVVYKDVITPEMKVGGDMVGAMAKAQQKLKTYAKDNGFKVTTDAD

SEQ ID NO: 44 - protein, 463 aa, Bifidobacterium catenulatum; identical to SEQ ID NO: 43

SEQ ID NO: 45 - protein, 325 aa, Bifidobacterium catenulatum; identical to SEQ ID NO: 42

SEQ ID NO: 46 - protein, 303 aa, Bifidobacterium catenulatum
MTNATAKPDTSGMRKPKREYIGILYCLPYVVVFLFGMIIPMFYALYLSFFKQSLLGGTTFAGFDNFLAAFKDEALWSGFRRVLIYAAIQIPLNLILSLAAALILDSQRIRHIAVPRILLFLPYAVPGVIAALMWGYIYGDKYGLVGQIASMIGATAPNMLSEKLMLFAIANICTWCSLGYNMLIYYSALIGIPNELYESARIDGASELRIAWSVKIPQIKGSIMMTVLFSVIGTLQLFNEPSILMNSAPNVITSNYTPNLYAYNLAFNGQNLNYAAAVSLVIGVIVMGLVAVVKVIGNKWENK

SEQ ID NO: 47 - protein, 461 aa, Bifidobacterium pseudocatenulatum
MKKKSVRLVAAIAALAMTAGISACGSDTNGSQAKSEVTAKEVESALTDTSKNVELTVWAYSAKQMEPTVKAFEEKYPHIKIKFVNTGAAADHFTKFQNVVQANKDIPDVVQMSANKFQQFAVSGALLNFANDSIDKAWSKLYTKTAWAQVHYAGGLYGVPQDATPLANYVRKDILDEHNLQVPESWEDIYNEGVKLHKEDSNKYMGILGSDISIFTNLYRSVGARLWKVNSVNDVELTMNSGKAKEFTEFLQKCLKDGVLEGGTVYTDEFNRSVNDGRYATFISENWMGNMYKDQNPSLKGKMVVTTPPSWNNQHFQSSSVGSMMSVAAACPKEKQAAALAFINWLDSAPDAIQSWQDTNSGNYFMAASVYQDDKTVREKKETDGYFANTDVNSVYFDSMDNVNTDWEYLPFMSQVEVVFNDVIVPEMNENGDLVGAMAKAQQKLKAYAEDNGFKVTTDAD

SEQ ID NO: 48 - protein, 325 aa, Bifidobacterium pseudocatenulatum
MSEAIAKPRSKSLQRRDAKLALKASKRYKRMQQREPAPKLTGKQRVLNWLLHIIMVLMVIYCLVPLLWVIFSSTKTSEGIFSSFGLWFDDKNVFWQNVQDTFAYQNGAYTRWLFNTIMYAAVAGVGATIIATFAGYAIATMRFPGRNALLAVTLAFMSIPSTVITVPLFLMYSKIGLVGTPWAVIIPQLATPFGLYLMIIYAQTSIPVSLIEAAKLDGASTWTIFWKVGFLLLSPGFVTVLLFTLVGVWNNYFLPLIMLSDTNDYPLTVGLNMWLKMGAQGTANGQVPNNLIITGSLIAVVPLIIAFMFLQKYWQSGLAAGSVKQ

SEQ ID NO: 49 - protein, 303 aa, Bifidobacterium pseudocatenulatum
MTNATAKPDTSGMRKPKREYIGILYCLPYVVVFLFGMIIPMFYALYLSFFKQSLLGGTTFAGFDNFIRAFKDEALWGGFKNVLIYAAIQIPMNLILSLVAALILDSQRIRHIAVLRILLFLPYAVPGVIAALMWGYIYGDKYGLFGQIAGMLDVTAPNMLSKQLMLFAIANICTWCFLGYNMLIYYSALIGIPNDLYESARIDGASELRIAWSVKIPQIKSTIVMTVLFSVIGTLQLFNEPNILRTSAPDVINSSYTPNIYTYNLAFNGQNVNYAAAVSLVVGIIVMALVAVVKIIGNKWENK

SEQ ID NO: 50 - protein, 473 aa, Bifidobacterium breve
MRTIGRRISRLATIVVAAVLTVSLASCGQATVDNRTEISVWSWEPSMGEVIRRFEKANPDIRVEWTNISGYENLNTAIQDGYGTPDVAQIEYYALPQYEVSGQLLDLTDRTGSDYASFFTLGTWASVQLAGRVYALPMDSGPMGFFYNEDVFQQAGVDATQIKTWNDYYEAAKKLKQIGAYIAADSGDGSFYDAMIWLAGGQPFHTSPDGKTVTIDLDHDTGTQLFTEFWQKMIDEGLVDTTSATWSERWKRRVGTGTIASMFSGAWMPSMLLSNVPGASGLWRVAPMPTYDGQPTNAESGGSALAVLQLTRKPDAAYRFVDFVTHDAQGISARVAGGAFPADYDTLNSGEFLDKTTITNDRGVEIPYFGGQKFNRVLSEAAEDVSVGYQYLPFEVYARNDFKSTVGKAYEWASSKQAYEQREMEVDMGMKDEKGKPLESLDRPGKKVSLEHGLATWQKDLKEYGFNQGFTIK

SEQ ID NO: 51 - DNA, 3472 bp, Bifidobacterium infantis
  source 1..3472: mol_type = other DNA
atgacaaatgcaacggcgcaaccggatacttccgtgatgcgcaagccgaagcgccaatacataggcatattgtattgtcttccatacgtggtggtcttcctgttcggcatgatcgtcccgatgttctacgcgctttatctcagcttcttcaagcagtcccttctcggtggcaccacattcgcgggcttcgacaacttcatccgagcgttcaaggatgaggctttgtggggcggctttaggaacgtgctgatttacgcggcgatccagattccgatgaatctgattctgtccctcgtggcggcgctcgtcttggactcccagaggatccgccatatcgcggtgccgcgaatcctgctgttcctgccttatgccgtcccaggcgtcatcgcggcgctgatgtggggatacatttacggcgataagtatggtctttttggacaaatcgccggtatgtttggcgtagcggctcctaatatgttgtcgaagcagctcatgttgttcgccatcgcgaacatctgcacttggtgcttccttggctataacatgctgatttattattctgcactgatcggcattccgaacgacctgtacgaatctgcccgtattgacggcgcttcggagctgcgcatcgcttggtccgtgaagatcccccagattaagagcaccatcgtgatgacggtcctcttctctgtgatcggcacgctccagctgttcaacgaaccgaacatcttgcgtacttccgccccggacgtgatcaacagcagctatacgccgaacatctacacctacaacctcgcattcaatggccagaacgtcaactatgccgctgccgtctctctggtcatcggcatcatcgtcatggccttggtcgccgtcgttaagatcatcggcaacaagtgggagaacaagtgatgagtgaagcaatcgcaagaccgcgttccaagtcgctgcagcgcagagacgccaaactggcgctcaaggcaagcaagcattacaagcgcatgcagcagcgtgagccggctccgaagctgaccggcaagcagcgtgtgctcaactggctgctgcacatcattatggctgtgatggtcatctactgcctcgtgccgctgctgtgggtcgtcttctcttccacaaagaccagtgagggcatcttcagctctttcggcctgtggttcgatgacaagaatgtgttctggcagaacgtgcaggatacgtttgcttatcagcatggtgtctacactcgttggctgttcaatacgatcatgtacgcagtggtagcaggtgttggtgcaaccatcattgccactttcgctgggtatgccatcgccaccatgcgattccctggacgtaacgccctgctggccgtcactctggcattcatgtcaattccttctacggtgatcaccgtgccgctgttcctcatgtattcgaagatcgggctggtcggcactccgtgggccgtgattattccgcagcttgccacgccgttcggcctgtatctgatgatcatctatgcgcagacctcgattccggtctcgctgatcgaagccgcgaaactggacggtgcaaacacttggaccatcttctggaaggtcggtttcccgctgctgtctccaggtttcgtcaccgtactgctgttcacgctggtcggtgtttggaacaactacttcctgccgctgatcatgcttacgaataccaacgactacccgttgacggtcggtctgaacatgtggctgaagatgggtgcccagggaacttcagacggtcaggttccgaacaacctcatcatcacgggctcgcttatcgcagttgttccgttgatcatcgcattcatgttcctgcaaaagtactggcagtctggcctcgcggccggctctgtcaagcagtgaaccttcggccgcacggtccctacgatcacccattcgttgccttggcgaccgtgcggcatccgccctttgggaaaacaacaaaatagtaattcatgacacacaaaggagtaatcatgaagaagagtatcaggcttgtggccgctatcgcggcgctggcaatgacggccggtatctcggcaagtggttccagcactaatgggaaccaggccaagagcgatgtgaccgcgcaggacgtcgaaaacgctctgaccgacacctcgaagaacgtcgaactgaccgtctgggcctattccgccaagcagatggagcccactgtcaaggcattcgagaagaagtacccgcacatcaaaatcaactttgtcaacaccggtgccgcggaggatcatttcaccaagttccagaacgtcgttcaagcccagaaggatattcccgacgtcgtgcagatgagcgctaataaattccagcagttcgcggtttcgggtgcattgctgaattttgccaacgatagcatcgaaaaggcatggagcaagctatacaccaagactgcatgggctcaggtgcattacgccggcggcctctacggtgcgccgcaggacgccacaccattggccaactatgtgcgaaaggatattctggacgaacataacctgcaggtgccagaaagctgggaagacatctacaacgagggcattaagctccacaaagaagattccaacaaatatatgggaatcctgggatccgacatctccttcttcactaacctctaccgttccgttggagcgcgtctgtggaaggttaattccgtggacgacgtcgaactgactatgaacagcggaaaagcaaaggaattcacggaattcctgcagaagtgcctgaaggatggcgtccttgaaggcggtaccgtcttcactgacgagttcaaccgttcgattaatgacggtcgttacgccacgttcatcaatgaaaactggatgggcaacacctacaaggaacagaacccgagcctcaaaggcaagatggtggttgctgccccaccgtcctggaaaggacagccttaccagtcctcttccgtcggctccatgatgagcgtctccgcggcttgcccgaaggagaagcaggcggcggctctcgcgttcatcaactggcttgattccgacaaggatgccatccagtcatggcaggataccaacaacggcaacttcttcatggcggcctccgtctatcaggacgatgagaaccagcgcaataagaaggagaccgacggttactacgccaatgatgatgtcaacgccgtctacttcgattcaatggacaaggtcaacaccgattgggagtatctcccgttcatgtcccaggtggaggtcgtgttcaacgatgtcatcgtgccggagatgaatgagaacggcgatctggtaggcgccatggccaaggcgcagcagaagctcaaggcatacgcggaagacaacggcttcaaggtcaccaccgacgccgactgacatccggttgcccgccattgctagggaggacggccaaaacggccgtcctccctttttgagattaaaccccggaaccaacgacgtggt

SEQ ID NO: 52 - DNA, 3481 bp, Bifidobacterium infantis
  source 1..3481: mol_type = other DNA
atgacaaatgcaacggcgcaaccggatacttccgtgatgcgcaagccgaagcgccaatacataggcatattgtattgtcttccatacgtggtggtcttcctgttcggcatgatcgtcccgatgttctacgcgctttatctcagcttcttcaagcagtcccttctcggtggcaccacattcgcgggcttcgacaacttcatccgagcgttcaaggatgaggctttgtggggcggctttaggaacgtgctgatttacgcggcgatccagattccgatgaatctgattctgtccctcgtggcggcgctcgtcttggactcccagaggatccgccatatcgcggtgccgcgaatcctgctgttcctgccttatgccgtcccaggcgtcatcgcggcgctgatgtggggatacatttacggcgataagtatggtctttttggacaaatcgccggtatgtttggcgtagcggctcctaatatgttgtcgaagcagctcatgttgttcgccatcgcgaacatctgcacttggtgcttccttggctataacatgctgatttattattctgcactgatcggcattccgaacgacctgtacgaatctgcccgtattgacggcgcttcggagctgcgcatcgcttggtccgtgaagatcccccagattaagagcaccatcgtgatgacggtcctcttctctgtgatcggcacgctccagctgttcaacgaaccgaacatcttgcgtacttccgccccggacgtgatcaacagcagctatacgccgaacatctacacctacaacctcgcattcaatggccagaacgtcaactatgccgctgccgtctctctggtcatcggcatcatcgtcatggccttggtcgccgtcgttaagatcatcggcaacaagtgggagaacaagtgatgagtgaagcaatcgcaagaccgcgttccaagtcgctgcagcgcagagacgccaaactggcgctcaaggcaagcaagcattacaagcgcatgcagcagcgtgagccggctccgaagctgaccggcaagcagcgtgtgctcaactggctgctgcacatcattatggctgtgatggtcatctactgcctcgtgccgctgctgtgggtcgtcttctcttccacaaagaccagtgagggcatcttcagctctttcggcctgtggttcgatgacaagaatgtgttctggcagaacgtgcaggatacgtttgcttatcagcatggtgtctacactcgttggctgttcaatacgatcatgtacgcagtggtagcaggtgttggtgcaaccatcattgccactttcgctgggtatgccatcgccaccatgcgattccctggacgtaacgccctgctggccgtcactctggcattcatgtcaattccttctacggtgatcaccgtgccgctgttcctcatgtattcgaagatcgggctggtcggcactccgtgggccgtgattattccgcagcttgccacgccgttcggcctgtatctgatgatcatctatgcgcagacctcgattccggtctcgctgatcgaagccgcgaaactggacggtgcaaacacttggaccatcttctggaaggtcggtttcccgctgctgtctccaggtttcgtcaccgtactgctgttcacgctggtcggtgtttggaacaactacttcctgccgctgatcatgcttacgaataccaacgactacccgttgacggtcggtctgaacatgtggctgaagatgggtgcccagggaacttcagacggtcaggttccgaacaacctcatcatcacgggctcgcttatcgcagttgttccgttgatcatcgcattcatgttcctgcaaaagtactggcagtctggcctcgcggccggctctgtcaagcagtgaacccttggccgcacggtccctacgatcacacattcgttgccttggcgaccgtgcggcatccatctgtttgaaaaaacaacaagatagtaattcatgacacacaaaggagtaatcatgaagaagagtattcgccttatagctgctgtagcagcattggccatgactgctggagcggctgcctgcggctctggtacttcgcagaaaaacaataaggctgatgtatctttgaatgatattaattccgctttaaccgatacctcaaaaacaacggatttgacggtatgggcatattcggctaagcaaatcgaaggtccggtcaaggctttccaggagagatatcctcatatcaaaattaacttcgtcaatacaggcgctgcatctgatcatttcacgaaattccaaaacgttgtttctgccaataaaggcgttccggatgtagtacaaatgtcgatcagcgaatatgagcagtatgcggttagcggagcgctcctaaactttgaatcagatgaaattgaaaaggcttggggaacgcagtatgcacaagcagcgtggaaaaatgtgcactttggtggtggactgtatggcaccccgcaggatgctgcacctttggccctatatgttcgaaaggacattctcgatgagcatgggctgaaagttcccactacttggcaagaattctatgatgaaggagtcaagcttcataagcaggatccaagtaagtacatggggttcatttcctctagcgatacttctttgttcggtgtattacgtaccgttggcgcaaagccttggacggtaaaggatacaacgaatattgatttctcgctaaccacaggtcgggtggcggagttcattaaattcattcagaagtgcctggatgatggagttcttcgtgctgctgctaccggtacagatgaatttaatcgtgaagtcaatgatggcgtatatgcaaccagattggaagggtgctggcaaggcaatatctacaaggatcagaatccttctttgaagggtaagatggtcgtcgcccatcctttggcatggggtaatgatggagaatcgtatcagtctgagtctactggctccatgttctctgtttcatcagctacaccaaaggataagcaagcagcggcattggctttcattcagtgggtcaatggcagcaaggatggagtttctgagttcttgacggcaaataaaggtaattactttatggcctctaactattatcagaaggataagagcaagcgagatcagcaagaaacagacggatatttcgcaaatactaatgtgaatgagatctattttgagtctatggataaagtgaacatggactgggattatattccattccctgcgcagctcacagttgcgtttggtgataccgtagctcctgctctgacaggtaagggagacttgttgactgcatttaccaagttgcaggataaccttaagtcttacgccgaggataacggttttaaagtcaccactgacgcagactgataaccgcccataatatgttgttgcggggaggatggctggtctgccgtcctccctttttgagattaaacatctttccaaggaagcgcact

SEQ ID NO: 53 - DNA, 3297 bp, synthetic construct
  misc_feature 1..3297: note = Codon optimized sequence encoding TP9
  source 1..3297: mol_type = other DNA
atgcgtaaaaagctggttggcaccctgctgattgcagcaatgaccgcaagcaccctggcaggtagcggtagcagcaatagcaccgcaggtacaagcgcaagcggtggtgcagcagaaaccagtccggcagaaagcgcaaccgaagccgaaggcaccgatgcagcagatggtgaagaggttctgaccgtttggtgttgggatccgacctttaatgtttatgcaatgaaacaggccgaagccatctatcagaaagatcatccgaacttcaaactggacatccaagaaaaagtgtataccgatatcgagcagaacattattaccgcagccgaagcagataattatgacaccctgccggatatttttctgatgcaggataatagctttcagaaaaacattgcgaactacccggaagtttttaccgaactgagcgatagcggtatcaactttgatgatttttcaggtggtaaactgggtgatagcacagccgaaggtaaacattatggtattccgtttgataacggtgcaagcattatggcaattcgtagcgatatggttgaagcagcaggtctgaccgtggaagattttaaagataccacctggtccgaatttatggaactggcaaagaaagttgttgatgcaaatggtgttccgatgctgaccagcagcggtggtagcgaaattgttattgaaatgctgcagagcgcaggcgcaagcccgattgttaatggtgaagttcatctggcagataatgcagcactgaaaaaggccattgaagtgtataaacagctgatcgatgaaggcatcatggtggattataccgattgggatcagtatattgccagcatgaataaaggtgaagcagccggtgttattcaaggttgttggattatgagcagcattcagagtgccgaagatcagagcggtaaatgggcaattgttaatatgcctgcactggatggtattgatggtgcaaccaattatgcaaattgtggtggtgcaagctgggcagtgagcagcaattgtaaaaatacagaactggccttcgacttcctgaaaaccacctttggtggtagtgttgaactgtatgatgatctgctgccgaatgccggtgcaattagcagctatctgcctgcagcacagagtgatgtttataatcagaccagcgatttttatggtggtcaggccgtttataaagacattgttgaatttgcaggtaaggtgcctgcaattaactatggtgcctattataacgatattcgcagcgcactgaccgatgcaattaccaatgttgttcagaatggtgcggatatcgacgaagaaattcagaatgcacaggatgcagtggaattcaacattagcgaataagtaagtgaaaaatgagccagtaccgggatcagcgtattgcgttcccggtattttttcacaggcgaggatgcacaggtttttacagaaatatatgtcgcccaaagcggcgtaaaagcggcgcagaatatcgcgaaacgatattctttttaatcaggagaatttatatgaaacagaaaaaggcgaagatgagcctggaaaagaaacataatctgaccggttgggcatttctgattccggcagcactgctgatttttatcttttgcttttatccgatgatccaggccattattctgagctttaccaagaaaggtagtggttttgcaggtctggcaaattatcagcgcatcctgaaagacaaaacctttcagcagtgtctgtttaataccgtgttctactttattatccaggttccggttatgctgctgctggcactgggtttagcccagctgctgaatagcccgaaaatcaaaggtaaaagcatttttcgcaccctgatttttctgccgtgtgcaaccagcctggttagctatagcatgatctttaaatcactgtttgcccaggatggtctggtgaataatatcattacaaccctgggttttgatcgcgttgattggtttcagaacgcatgggcagcacgttttgttattatcattgcactgatttggcgttggaccggctataatatggtgttttatctggcaggtctgcagaatattgatgatagcgtttatgaagccagctatatcgatggtgcgaccccgttccagcagtttatgaaaattaccattccgctgctgcgtccgacaattctgctgaccgcaattatgagtaccagcggtacactgcagctgtttgatgaaagcgtgaatctgaccagtggtggtccgggtaaaagtaccatgacactggcacattatgtgtacaatatcagctttgttgaaaccccgaaattcaattatgccgcagcaattagcgtatttatccttgtgagtgtggcagttctttctgcaatccagatgaaagtaggtgacaaacgtgactaaagtaaagacaactatcgcttatattattttaatcattgccagtttcctgtcagcgttcccgctgtattatatgatttgtggtgccaccaatagcagcattgatattgttcgcggtaaactgtttccgggtgtttatctgatggaaaactatcagagcctgattgcgaatcagaatctgggtcgtgcaatgtttaatagttttcgcaatgccattgtgattaccgttattgcgctgctgatctgtagcattgcaggttatggttttgagatctatcacgacaaaggcaaagatctgctgatgaacattctgctgttagcaatgatgctgccgtttgttgccattatgattccgctgtttaaaatgtttagcagctggaaactggtgaacacctggattgccctggcactgccgagcctgagcaccccgtttctgattatgatgtttcgtcaggcagcccgtagctttccgcatgacattattcaggcagcgcgtctggaaggtctgagcgaaattcgtattttctttaccatgtttctgccgatcatgaaaagtacctatggtgcagccatgacagtgacctttatgaatgcatggaacaattatctgtggccgaccatcattctgcaggatagcaatcagattacaatgccgatgctggttgccaatctgaaaagcggttatagcgttgattatggcatgctgatgctgggtgttctgatttgtaccctgccgaccgccattatctttctgtgcctgcagaaaagctttgccaatggtattgcgggtgcagtgaaataatgaatcggaaatattgtctggataaaaaagaatgggaggaggcgcaggcagcccttcttcttgcaaaacagttcgggctcattgaagatg

SEQ ID NO: 54 - DNA, 3515 bp, synthetic construct
  misc_feature 1..3515: note = Codon optimized sequence encoding TP18
  source 1..3515: mol_type = other DNA
atgaccaatgcaaccgcaaaaccggataccagcggtatgcgtaaaccgaaacgtgaatatattggcattctgtattgtctgccgtatgttgtggtttttctgtttggtatgatcatcccgatgttctatgcactgtatctgagctttttcaaacagagcctgttaggtggcaccacctttgcaggttttgataactttattcgtgccttcaaagatgaagcactgtggggtggctttaaaaacgttctgatttatgcagcaattcagatcccgatgaatctgattctgagcctggttgcagcactgattctggatagccagcgtattcgtcatattgcagttctgcgtattctgctgtttctgccttatgcagttccgggtgttattgcagccctgatgtggggttatatctatggtgataaatatggtctgtttggccagattgcaggtatgctggatgttaccgcaccgaatatgctgagcaaacagctgatgctgttcgcaattgccaatatttgtacctggtgttttctgggctataacatgctgatctattatagcgcactgattggtattccgaacgatctgtatgaaagcgcacgtattgatggtgcaagcgaactgcgtattgcatggtcagttaaaattccgcagattaaaagcaccattgttatgaccgttctgtttagcgttattggcaccctgcagctgtttaatgaaccgaatattctgcgtaccagcgcaccggatgtgattaatagcagctataccccgaacatctatacctataatctggcctttaatgggcagaatgttaattatgcagccgcagttagcctggtggttggtattattgttatggccttggtcgccgtcgttaagattatcggcaacaagtgggagaacaagtgatgagtgaagcaatcgcaaaaccacgttccaagtcgctgcagcgcagagacgcaaaactggcactgaaagcaagcaaacgttataaacgtatgcagcagcgtgaaccggcaccgaaactgaccggtaaacagcgtgttctgaactggctgctgcacattattatggttctgatggtgatttattgtctggtgccgctgctgtgggtgatttttagcagcaccaaaaccagcgaaggtatctttagcagctttggtctgtggttcgatgataaaaatgtgttttggcagaacgtgcaggatacctttgcatatcagaatggtgcatatacccgttggctgtttaacaccattatgtatgcagcggttgccggtgttggtgcaaccattattgcaacctttgccggttatgccattgcaaccatgcgttttccgggtcgtaatgcactgctggcagttaccctggcatttatgagcattccgagcaccgttattaccgttcctctgtttctgatgtatagcaaaattggtctggttggtacaccgtgggcagttattattccgcagctggcaaccccgtttggtttatatctgatgattatctatgcccagaccagcattccggttagcctgattgaagcagccaaactggatggtgccagcacctggaccatcttttggaaagttggttttctgctgctgagtccgggttttgttaccgtgctgctgtttacactggttggtgtttggaataactatttcctgccgctgattatgctgtccgataccaatgattatccgctgaccgttggtctgaatatgtggctgaaaatgggtgcacagggcaccgcaaatggtcaggttccgaataacctgattattaccggtagtctgattgcagttgttccgctgatcattgcatttatgtttctgcaaaaatactggcagagcggtctggcagcaggtagcgttaaacagtaaaccctcggccgcacggtccctacgatcacccattcgttgccttggtgaccgtgcggcatccgtccttttgaaaaaacaacaaaatagtaattcatgacacacaaaggagtaatcatgaaaaagaaaagcgttcgtctggttgccgcaattgcagcgctggccatgaccgcaggtattagcgcatgtggtagcgatacaaatggtagccaggcaaaaagcgaagttaccgcaaaagaagttgaatcagcactgaccgataccagcaaaaatgttgaactgaccgtttgggcatatagcgcaaaacaaatggaaccgaccgttaaagccttcgaagaaaaatatccgcacatcaagatcaagtttgttaataccggtgccgcagcagatcactttaccaaatttcagaatgttgtgcaggccaacaaagatattccggatgttgttcagatgagcgccaataaattccagcagtttgcagtttcaggtgcgctgctgaattttgcaaatgatagcattgataaggcctggtccaaactgtataccaaaaccgcatgggcacaagtgcattatgccggtggtctgtatggtgttccgcaggatgcaacaccgctggcaaattatgttcgtaaagatatcctggatgaacataatctgcaggttccggaaagctgggaagatatctataatgaaggcgtcaaactgcacaaagaggacagcaacaaatatatgggtattctgggtagcgacatcagcatttttaccaatctgtatcgtagcgttggtgcacgtctgtggaaagttaatagcgttaatgatgtggaactgacgatgaatagcggtaaagcaaaagaatttaccgagttcctgcagaaatgtctgaaagatggtgttctggaaggtggtacagtttataccgatgaatttaaccgtagtgtgaacgatggtcgttatgccacctttattagcgaaaattggatgggcaacatgtataaagatcagaatccgagcctgaaaggtaaaatggttgttaccacaccgcctagctggaataaccagcattttcagagcagcagtgttggtagcatgatgagcgttgcagcagcatgtccgaaagaaaaacaggcagcagcactggcgtttattaactggctggatagcgctccggatgccattcagagctggcaggatacaaatagtggtaactattttatggcagccagcgtgtatcaggatgacaaaaccgttcgtgaaaagaaagaaaccgatggctattttgccaataccgatgtgaatagcgtgtatttcgatagcatggacaatgtgaataccgattgggaatatctgccgtttatgagccaggttgaagttgtttttaacgatgttattgtgcccgagatgaatgaaaatggcgatctggtgggtgcaatggccaaagcacagcagaaactgaaagcctatgccgaagataacggttttaaagttaccaccgatgccgattaacatccggttgcccgccaccgctagggagggcggccgaatggccgccctccctttttgagattaaacatttttccaaagaaggaagcgcatcgtgagcgttattaccaaagtcaccacctatgattt

SEQ ID NO: 55 - DNA, 5108 bp, synthetic construct
  misc_feature 1..5108: note = Codon optimized sequence encoding TP11
  source 1..5108: mol_type = other DNA
atggcaagcgcagatgttgcagcagttccgatgcataccgataaagcacgtgaaaataaacacaaagcagattggcgtggctggaaatttatgtggccgtttaccgttgtttttgtgttcgtttttatcatcccgattgtgtatgccgtgtatatcagcttctttaaaaagcagatgattggtggcacccgttttgttggtgttgaaaattatgcacgtctgctgcaggatggccagttttggagcagcgttggtcgtgttgcactgtttacctgtgttcaggttccgattatgctgtttctgagcgcagcaatggcactggcactggatagcatgaaactgcatggtgcaaaattctttcgcattagcacctttctgccgtatgcagttccggcagttgttagcaccctggtttggggttttgtttatggtgccaaatatggtctggtgggtagctttaatgatttctttggtacaaatgtggatgtgctgcagcctggtgttctgctggcaagcattggtaatatttcaacctgggaatttaccggctataacatgctgatcttttatagcagcctgagcaccattccgcatagcctgtatgaagcagccagcattgatggtgcaagcgaatggcagattgtgaaaagcattaaactgcctgagctgaaaggtagcctggcaattaccgtgatttttagcattattggtagctttcagctgtttaacgaaccgagtatcctgcagaatatggttccgggtaatgcaattaccacctattataccccgaacatgtatgcctataatctgagctttagcggcaatcagagcaattatgcagcagcactggcaattgttatggcagttattaccatggcaattgcatatgccgtgcagctgaaaagtatgaaagaacaaatgaaataacgacgaccgcccgtattgccggattggtttcgattgtgaaggagcgtgcgtgatgtcagcagcagcaagcacccgtctgaccgatgcagaactgaaagctcgcgaaaaagcagcacgtaaagcagaaaaagaacgtcagaaacgtattgcagcagatgaagccgcagaacgtaaacgtagcgcacgtgcaggttttgcaaatgttaataatccgcgtcgtagtaccctgatgaccgttctgtgtgcaatttttgcagtttattgcctgtttccgtttgtgtatctggtgattaatgcaaccaaaacgcaggcagattttaccagcacctttggtttaggttttggtaaaagctttgccctgtgggataatattgttaccgtgtttacctatcaaggtggcatttttggtcgttggctggttaataccctgctgtatgttgttgttggtgccggtggtgcaaccctgctggccattatgggtggttatgcactggccaaatttcgttttccgggtcgtaaagccgtttttgccgttattattggttcaattagcgttcctggtattgcactggcggttccgcagtttctgctgtttgccaaactgggtctgaccaatacaccgtgggcaatgattattccgagcctgattagcccgtttggtctgtatctgatgtggatttttagtgaacaggccgttccgaccgaactgctggaagcagcccgtgtggatggtgcatcagaatttcgtaccttttggaccattagcctgccgctgctggcaccgggtattgtgaccactgcgctgtttaccattgttgcaacctggaataactattttctgccgctgatcatgctgaaagatgcagattggtatccgctgaccattggtctgaatcagtggaaagatcaggcaagcaccgcaggcggtcaggcaattcagaatctggttattaccggtagtctgattaccattattccgctggttattgcatttctgtgcctgcagaaatattggcagagcggtctggcagccggtgcagttaaagaataaccgtacttcggctcccttctggtggagggagccgagataaaggcataacacaaaacagtttgaatcgttctagacgattcgaggaaggagaacagaacgatgaaatttaccaccgcaaagaaagcagttgcactgaccggtgccgttgcaatgctggttagcgttgcagcaagcggtagcgatagcggtaaaagcagtcagcctgcacaggatagtgatgttaaagaaattaccgtttgggcatgggaaccgagtctgacccaggttgccaaagattttgaaaaagaaaccggcatcaaagtggacctgaaaaatgttggcaccaataccaaagagtatacccagctggataatgccattgaagcaggtagtggtgcaccggatgttgcacaggtggaatattatgcagtgccgcagtatgcaattaaaggtaatctgctggatatcaccgataaaaccagcggctataaagatttctatacccctggtccgtgggccagcgttcagtttgcaggtaaagtttatggtctgccgatggatagcggtccgatggcattcttttataacaaagaggtgtttgacaaagccggtgttgatgcagagcagattaaaacctgggatcagtattatgatgcagccaaaaagattcatgccctgggcgataattactatattaccagcgataccggtgatgcaggctttttcgatagtatgacctggctggcaggcgcaaaaccgtttcagaccagctcagatggtagcgaagttaccgttaatctgaccgaagataaaggtgtgaaaacctttaccgatttctggcagaaacttctggatgaaggtctgctggacaccaaaacagccggttggagcgaagattggtttaaaggtatggttgatggcaccattgcaagcctgtttaccggtgcatggatgcctgcaaatctggcaaatagcgcagccgatggcgcaggtaaatggcgtgttacccagatgccgaccgcagatggttcaaccaccaatagcgaaaatggtggtagcagtctggcagttctggccagcaccaaaaaggcagatgcagcatatcagtttatcgaatatgcaaatcatggtgcgggtgttgcgacccgtgttgcaggcggtgcctttccggcagataaagcaagcctggaaaaagatagctttaaaaacgcaaccaccgtgaaaaatgccgatggtcaggatgttgattattttggtggtcagaaatataacgaagttctggcacaggcagccgaaaatgttagcagcggttatcagttcctgccgtttgaagttaaagcccgtaccatttttggcgattatttcggcaaaagctatacgggtgatcagaaactgagtgatggtgttgccgcatggcagaaagcactgcaggattatggtaaagatcaaggctttaccgtgaaataagccttataattccttctctatatatccacagcagccccgtgacgcatctcgccacggggctgctgccatattggctcccgctggcaggagctgactcgcgaggcgagactgagggtggttcatcatacggcgttttaagccccgcctagtagactcgtcaccatgcgtaccattggccgtcgtattagccgtctggcaaccattgtggttgcagccgttctgaccgttagcctggcgagtagtggtcaggccaccgttgataatcgtaccgaaattagtgtttggagttgggaacctagcatgggtgaagttattcgtcgttttgagaaagcgaatccggatattcgtgttgaatggaccaatattagcggctatgaaaatctgaataccgccattcaggatggttatggtactccggatgtggcccagattgagtattatgccctgcctcagtacgaagttagcggtcagctgctggatctgacagatcgtaccggcagcgattatgccagctttttcaccttaggcacctgggcaagtgtgcagctggcaggtcgtgtttatgcattaccgatggattcaggtcctatgggctttttctataacgaggatgtttttcagcaggcaggcgttgatgctacccagatcaaaacatggaacgattattatgaagctgccaagaaactgaaacaaatcggtgcatatattgccgcagatagtggtgatggtagtttttatgatgctatgatttggttagccggtggtcagccgtttcatacctcaccggatggtaaaaccgttaccattgatctggatcatgataccggtacacagctgttcaccgaattttggcaaaagatgattgatgaaggcctggttgataccacctcagccacttggagtgaacgttggaaacgtcgtgttggtacaggtacaattgcgagcatgtttagtggtgcctggatgccgagcatgctgctgagcaatgttccgggtgccagcggtctgtggcgtgtggcaccgatgccgacatatgatggccagccgaccaatgcagaaagcggtggtagcgcactggccgttctgcaactgacccgtaaaccggatgcagcctatcgctttgttgattttgtgacccatgatgcacagggtatttcagcacgtgttgccggtggcgcatttcctgccgattatgataccctgaatagcggtgaatttctggacaaaaccaccattacaaatgatcgcggtgtggaaattccgtattttggcggacagaagtttaatcgcgttctgagtgaagcagcagaagatgtgagcgttggctatcaatatctgccgttcgaggtttatgcccgtaatgactttaaaagcaccgttggtaaagcatatgaatgggcaagcagcaaacaggcatatgaacagcgtgaaatggaagttgatatgggcatgaaagatgaaaaaggtaaaccgctggaaagcctggatcgtccgggtaaaaaggttagtctggaacatggtctggcgacatggcaaaaagatctgaaagaatatggctttaaccagggctttaccattaaatgactcctcgctaacctccctctgacgagggaggtggccgccgaaggcggacggagggagagacctgagggaagtcctagctaaaggccgatttcgtcacttc

SEQ ID NO: 56 - DNA, 3529 bp, synthetic construct
  misc_feature 1..3529: note = Codon optimized sequence encoding TP8
  source 1..3529: mol_type = other DNA
atggtgagccataataaacgtattgttgcagcatttgcagcagttgccgcaatgggtatgggtttagcaggtagcggtggtagcgataccgcaggcgataccaaaagcggtagtgatggtggtgttgtgaatatcacctatatgcatcgtctgccggatagcaaaggtatgaccctggttaatgatattgtggccaaatggaataaagagcatccgaacattcaggtgaaagccaccaaatttgatggtaatgcaagcgagatgatcaaaaagctggaaaccgatgttaaagcaggtaatgcaccggatctggcacaggttggttatgcagaagttccggaagtttttaccaaaggtctgctgcaggatgttaccgatgaagcagcaaaatacaaagatgattttgcaagcgcaccgtttgcactgatgcaggttgatggcaaaacctatggcctgccgcaggataccggtccgctggcatatttctataatgcagcagaatttgagaaactgggtattaccgttccgaaaaccgcagatgaactgattgaaaccgccaaaaagaccgcagcacagggcaaatatatcatgacctttcagccggatgaagccatgatgatgatgtcaggtcaggcaggcgcaagcggtccgtggtataaagtggatggtaatagctgggttgttaacacacagaccaaaggtagcaaagcagttgcagatgtttatcagcagctgattgataataaagcagcactgaccaatccgcgttgggatgcaagctttgataatagcattcagagcggtcagctgatcggcaccgttgcagcagcatgggaagcaccgctgtttattgatagtgccggtggtacaggtgccggtgaatggaaagttacccagttaggtgattggtttggtaatggcaccaaaaccggttcagatggtggtagtggtgttgcagttctgaaaggtagtaaacatccagcagaagcaatggaatttctggattggtttaatacacaggtggatgatctggttagccaaggtctggttgtggcagcaaccacagcagatgccaaaacaccgcagaaatggtcagattatttcagcggtcaggatgtgatggcagaattcaaaaccgcgaatgataatatgggcgattttacgtatatgcctggttttagcgcagttggtgcagcaatgaaacagacagcagccaaagcagcagatggtagcgcaaaagttagtgatgtttttgataccgcacagaaaaccagcgttgataccctgaaaaatctgggtctgagcgtgaaagaataacctcactaggtcttgtcaacgggccggcttgaaatagccggccatccaagacctccgatgcctcgcatcaccatagcgaaaggccggggcacatactccggcctttcgcattgtttgagggtattgctgcaagggcgctgtggcccttgtcatttgttgtactgttctttgcaaagacaggttttgtattatgaccgcaagcaccacacatgcaaaaggtaaagcaccgcgtcgtgcagccgcaaaaccgaaacgtagcggtgcacagaaacgtgaaaatcgtaccggttgggcatttatggctccgtttgccgttctgtttgcctttgtttttattctgccgattatctgggccatctatagcagctttttccgtcaggttaccgaaggtggtggtgcatatggtggtggcgaactggtgaataaattcgttggttttcagaattttcagtatgtgattaccagcggcaatttttggagcggtgttggtcgtgttctggtttataccctgattcaggttccgattatgattattgcagcactggcactggcaattgtgattgatagctttgttgttaaacacgtgaccggttttcgtctgggttatttcctgccgtatgcaattccgggtgttgtggcaagcattatttgggtgtatctgtataatggccagattagcccgattgttaaaggtctggcagcaattggtattaacgtggatttctttagcagcaacattgttctgggtagcatggcaaatatcaccacctggacctttaccggttataacatgctgatttttctggcagccctgcaggcaattccgcatgatctgtatgaagccgcacgtattgatggtgcaaatggttggcagattgcaaccaaaatcaaactgccgaatgttcgtggtgccgcactgctggctatgctgctgagcattgttggcaccattcagctgtttaatgaacctcagattatgagcaccgcagatccgggtattagcaaagcctatacacctatgatgatggcaatgaataccagccagggtacaattaccccgagtggtgatggtccggcaagcgcaattgccattgttatggcactgattgccggtgtgctcgctgtgctgtacaccctggccgaaaggaaggtgaacgagtgatgagctccgcaaccgctgaagaacgcgcccgcaggaaggccgagcgcaaactggaaaaacagcgtcaggcagcacagtatgatctgcctcgtagcatgcgtccgagcgttgcgaccaaaaccattaccattattgtgctggttctggtgctgatctattttctgtttccgatttattgggcgattatcgccagcacaaaaacaccggcacagatgaccggtagtaatggtatgtggtttgcggttccgctggatcagctgccggatgccattgccaccaattatagcaaactgttaggttggacccgtggtaatttttggcgttgggttctgaatagcctgatttatagcggtgtgagcgcactggttggtacaattgttagcgttatggcaggttatgcaaccgcgaaatttaactttcgtggtaaaaatgcagccatcggtattatcatggcatgtatgctgatgccgagtgcactgctgaccattccgatgtatagcatttttcataccctgcatctgaccaataccatgctgagtatcattattccgtgttgtgttagcccgtttggtttctttctgggtcgtacctatgcacagagcagcgttccggatgaactgctggaagcagctcgcattgatggcgcaagtgaagcacgtattttctttaccattgtgctgcgtctgctggctccggcaatggttaccattttcctgtttctgtttgttgccacctggaataactttctgctgccgctgatgatggttagcagcgatacactgaaaccggttacactgggtctgtatggtatggttagcctggcaacctttacagatcgtggcgcattaatgatgggtgccctgctgggtgttctgccggttattgttctgttcctgggcctgcagcgctattggcagagtggcctggcagccggtgcagttaaaggttaaccaatccgctgcccggtatgtcgtgtcactccttccggtgccgggcaacgtccccctaccctcattaccagtgacacgattccaatcgtccggcgtcacc

SEQ ID NO: 57 - DNA, 4761 bp, Bifidobacterium breve
  source 1..4761: mol_type = other DNA
atgacgttcgttgcacggtcggtgcgctatgtgctcagaaaacgagtgcgaaccatagtgctcctcatagtactgaccatcataacggcgagtatgctatccacgatcgcggtttctcgggcagctcaacaggccgccggacgaatcgagaaggaagcggtcggtggattcgtgctggccagcaacctgcagggcagcatgctcacgcctcgcggaggcggcatggtgcgtcccgccgacgtgcgccggatcgcccaactgcccggggtcgattcgtatatggtccgtcagaacgccaccgccgatctcgtgggggcgaatgtggcgaaggtccccggcggcgatgattacgatgcgaccaaggagcagcagttcggcaacgccgccaacgtcataggcacgaatgattcgtcggaactcaacgtgttcacgtcgcacacgctcggcatggccgaaggacgacatctgaaggcctccgacaagtacacgtcgatgatccatgaggatctcgccaaagccaacgggctcaaagtcggagacacgctcacgctcaaggccaacgcctatgacgccgacaacgaatcacattcgacggcgaccgtcaagaccaccatcgtgggcatattcaagggcgacagcgccagaaaggtatccagccgagcggaactcaccgccaacaccatctacaccgatctggataccacacgcgatctgtaccagtacaaggacggcaaggaaatataccaggacgccaccttcgtgctgtccaagggcgttgacgtggagaagaccatggatgccgcgaagaaactcccggtggattggaacaactatcagatcacgcgcaacgaccagtattcgtcgagcatgctgcatgccgcccgcggcgtgcgctccatgatgcgcggcgccctgatcggcgtgaccgtctcggccgtgctcgtgctcagtctcatgctgctgctgtggatgaacgaccgccgtcaggagatgggcatactggtgtcccttggcgtcggcaagccctccctggtcgcccaatatctcgctgagatggtgctgatcggtctgccgtcccttgccctcggctggctgtgcgcgcagggaatggcccagtggctcggcaccacggcgctccattccgtgaacgcctccgcggcgaaggagctgagcagcatggggcaggtcggcggcgatcttgaatccagcatgtcggtgcgcaccctggattcgctcacggtgtcggtcgacgggaccgccatgctgtacgtggcgctcggactgttggcggtgatgctggtgtgcgtcgcggtggcgtgcatgccgatgctgcgcaaatcgccgcgagacctgtcggagattcgatgatgagcatgaatgtgtggaagcgtgccacgttggcgatcgtgcgcaagccggtacgcagcggcatcatcggtctgctgatgctgatggtcttcacgagcctggtggcgcaggtcggagtctcgacggccctgcgtaccatgtccgacagcatcggcgcgggcatgggcatagggtttaccgtcagcgccggcgagaatcccatcagcgccgaggaggcgagccggttctcgcgtatccccggtgtgacgaagaccgcctatgcgacgaagaccttggcgcaggtggatggcgcgcgtccggtcatgccccggcagggcccccgactggactccgatctggccatgcaggtcagcgtgctgggcaccaccgattcctcattgtccgaggaattccagagtggcctgtaccgcttggagcagggccgccacatctcgggcgacggcgacaatgtgctggtccaccgcgacttcgcgctgcagaacggcctatcggtcggctcgacattccgtctccggcaggaggggcgcaacgccacggtgcgtgtggccgggatcttctccggcaacgtgcaggcccaaagccccctgccctcggacacttccgagaacctcatctattcgggcaggcgagtgacctccgctctgaccggaaacgaccagatcggcatgatccgatgcctgtcggataatccgcaggacctgtccgcggcggtcagtcaggccaagaccatggccggcggcaagtacgacgtcaccgacgattcggcccggctctccggcgtcctgcaatccgtgcagacggtgcgtgatctggtgcgcatggtgctgctgtccgtctgtctcgcggacgtactggtgctggcgatggcgctggtcttctggattcgttcacgcatccatgagataggcacgcttcttgccctcggcatcggcaagatgcggattgtcgcacaactcgccatcgaaaccggactcatggcggcggtcgccgccctgtgttcgctgggcaccggcgccatgctgtcgggatacgtgtcttcccgactgttgcgcgattcgggcgtcgcgccgctggaatcgttgcatgtggaggccttgccgcctgagcagacgatgctcatcctgctgctcggatgcgcggtcatcgccgtggcccttgccgtgtcatgcgccgccgtgctgtccaaatccccgaaatccattctttcctctatgcgttaggagtcaccaatcatgtctaactgtctgcaactggaccacgtcgattacgcctacggcgcggcgaagatccttgttctttcggatgtcagcgcggatttcgaatccgggaagatgtatgcgatcaccggcccgtccggcgccggcaagagcacgctgctgtcgctgctggccggcctggacgccccctcccggggagtggtccgcttcgaaggggaggacatcgccgcatcgggctatgcgaagcaccgtcgggagcacgtgtccctggtgttccaggaccataacctgatcgactatctcacccccgaagagaacctgcggctggtcagcaccaaggccgatatgaagatcctcgaggaactgggtttgagccgtgaggaatccaagcgcaacatcatgcacctgtcgggcggccagcgtcaacgggtggcggtcggccgcgcgctggtcgcgccgggccgcgcgattctcgcggacgagccgacgggcagcctcgacccggagatgaccgacgaggtgattcatctgctgcagcacgccgcccatcagatcggcaaatgcgtgatcgtggtgacgcattccaagcgcgtcgccgactccgccgatgtcgtgctgcgtctcaggagcaagaaactggcccgggcgtgacccgcctgggccgggcggcacccatacaccgctccggcaccccggcgacccactcgcgttgccgggggccgtattcgttctgcgccataatgtgcgtgaatgaacaacaccacgcgcgcagtcatagccacggctgccatcggagccctgctcatccctctcgcgggaagcggcaacacgaccacaagctcgggcaagaccgaactgacggtatggtcgtgggacgccagcatcgatcgcgccgccaaatccttcatgaaggccaatcccgacatcacggtgaaggtgagcaacgtcggcagcaccgaggagacctacaccgcactcaacaacgcggcccaagccggcagcggtctgcctgacgtcaccctgatcgaatacctcgccatcccgcagttcgtgcattcggacacgctcatggacctgagcaaggtctacaacaccgccaagctcaaggacacattcacccccggcacatggaactccgtgaacatcaacggcggtctgtacgcgatgcccgccgactcgggccccatggcctacttctacgacaaggacgtgttcgacaaggccggcatcagcgaaccaccgaccacctgggacgagttctacgaagacgcgaagaagatccgcgccaccggctcctacatcacctccgactccggcgacgctggcctgttcaacgcaatgatgtgggccatgggcggccacgcctacaaactcaacgacgacaaactgaccatcaatatcgccaaggacaaaggcgcccagcggttcatggacctctggcagaagatgcgcgacgaagacctcatcgacgtccacacgaagacctggaccgacgactggatgaagtcactgggtgacggctccatcgccagtctcatcagcggcgcatggatggccaacaacctcctgcagggcgtgccccaagccaccggcaaattccgcgtagccctcctgcccaccatcgacggaacgcccatcaacggcgaatacggcggcagcggactagccatcaccaagaagatccccgacggcaaactcgacgccgccaagaagttcgtcgaatacgtcaccaccaacgacgaaggcatcgacgcccgcgtatccaacggcagcttccccgccaccaccaagaccctcaaccagaaggacttcctcgccaagaccacgctcaggaacaccgacggcagcgacaacgagttcttcggaggacagaaatacaacgaggtcttctcccaagccgccaaggatgtcaccggcaaatgggagttcctgccctttgaaccgtacgcacgatccgtctacaacgacaacatgggatcgttcttcgcgggcaaaacctccttcagaacagccgcagacaactggcagaaggctctaaaggattacgccaaggatcaaggcttcaccgttcagtgaccaataatccaaacggtgaataacgtcggtatcttccgtcgacaaagataggcaccctaccccaatctccttccggggtagggtgcttttgcattcatta

SEQ ID NO: 58 - DNA, 3622 bp, Bifidobacterium infantis
  source 1..3622: mol_type = other DNA
atgactcacttcagcactgtgaaacgcgtcatcgctctcgccggtgccggtgccatgctgttttccgtggcggccagcggtggggtaaccgcttccgatggcggcaagaccacactcaaattcgcggcgttcgagggcggctacggtgccgacatgtacaaggaggtggtcgccgcatacgagaagctcaatcccgacgtcaagatcgaactgaccacatccaagaagatcgaggacgagatcacgccgggcatgaaggccggcaactatcccgacatcgtggagctgggacagggctcgacctccggactgaccgagacgctgctcaaggacaaggccatcgaggacgtgaccgatgtgcttgacatgaaggtccccggggaaaacaagaccgtcaaggacaagctcgtcgacggcgtgatcggcctgtacaccaacccctacggcggagacaagacctacctcatgcccatgtactactcaccctccggcttggtgtacaacaagacgctgcttgaacaaaacggctggaagatgcccaccacatgggacgagatgttcaagctgggcgatgaggccaaagccaagggcatctcgctgttcacctacccgaccgcaggttacttcgacgccttcttcaacgcgctgctcgccgacatcggcggcgaccagttctaccaggacgtgatgacctacaagaaggacgtgtggaagaccgacgaggccaaggaggcgttggagaccacctataagctggtcaccgaatacctcaaccccgacaccgtgggctacgccaacgcccaggacttcaccaagaaccagcagtccatcctcgacggcaagtccctgttcatgcccaacggcacctggattgtcaacgagatgaaggatgcgccgcgcaccagcggcttcgagtggggcttcgccccggtgcccaccgtcaagaatggcggcacccgctacatcaacaccacaatcgaagccgtgtggattcccgccaaggccaagaacaaggaagcggcgaagaagttcatggcctacctgtactccgacgaagccgcgtccatcttcgccaagaccggtgccatccagccgatcaagggcaacaccgatgacctggcctccgacatgaaggtgttctacgacgcctacaacgaggacaacgtcaaggccgtggccggctcgttctccgcaaccgccaccgtggaaggcaagaacatcaaggacgacctgtacaacgcggtcgactccgtggcctccggcaagaccacgctccagcagtggcaggacaagctcaacgagaccagcaacgcactgaacgcggcggccaagcagtaacctcgccgtcgaacaacggccggagcagatggtcccacggtccggcccgtcgcaccgatgcattcatcggcggcgggcggggcggcggaccgctccggccccgctttgcctcagcgccatacaggcaacgcttttcagggggaacgatgtcttcaacgaaacatacaaccgaccttggcaggatgcgcagccgtttcggcattctttgctcgctgcccaccgcaatcctgctcatcatcttcatcttcatacccacggtcaacgtgttccgcatgtccgtatacaaatggagcggctactcggacgaacagaccttcgtggggctttcgaacttccggaaactgttcgtcgatccgaacttcatccacgcgttccagaacaccgtgctcatcctcgtgctggtcacctccgtcaccatgccgctggccatcatcttcgccgcggtgctcacgcagaaccgcatcatcggaggctcgctgattcgcttcatcctctatgtgccctcggtgctgtccatcgtggtggtcgcggccatattctccgccatctacgatcagaacaacggcctgatcaacggcaccctgagcttcctgcacctgaacgggctcaagcaggtgtggctgggcaatcaggacatcgtcatcttctccatcgccttcgccatgctatggcagtcctgcggatactacatggtgatgtacatgtcgtcgatggcgggcatcgacccgagcctgtacgaggcggcgcagctggacggcgccagcaagactcgacagctgttctcgatcaccatcccattgatctgggcgaacatccgcaccacgctcaccttcttcatcatcagttcggtgaacctcagcttcgtgctcgttacggccctgaccaacggcgggcccgataacgcgtctcaagtgctgctcggctacatgtacgaccaggcgtattcgaactcgtcctacggctacggcatggcgatcggcgtggtgatcttcctgttcagcttcatcatgtcgttgatcgtgaaccgtgccaccaagcgcgaagtgctgcaatactaaaggagggcgagatatcatggcttattcatcagtttccggaagaccggcccactccaggcgcgtgcgtccggcggccgatcgaggttcggcccattcatggacggcgggccgcatggttggccaggtggtgctggtaatcgccgccttgggcatcgccatccccaccctgtgggtgttcgtggcttcgttcaaggataaaagtgagttctacggatcaccctggacctggcccaagggcctgcactggcagaactatgcggatgctttcgtggacgccaagctcggacagtacttcggcacatcggtgctggtcaccgtcatgtcgatggccatggtgctggtgatcgcgctgccctgctcctacgtgctcgcgcgcttcgattttccgggcaagaagattcttaaactcgccgttcaggccggcctgttcatcaacgtgaactacatcgtcgtgccgatcttcctgatgcttgtggacgccgacaagctgctctacaacatcatgccgaacgggttcttcgtgaacaacgtgttcatcctggcgctggtctacgcctcgacgtcgctgccattcaccatcttcctgctccaggacttcttcgcctccattccgaaggactacgaggaggccgccatgattgacggcgcctcgcaaaccaggatcatgacccgcatcttcttcccgatggccatgcccgccatctccatgtcgatgctgttcaacttcctgacctactggaacgactacatcatctccatgaccctcatgaccggcaacaaccgcaccgtgcaagtgggcctgctcaacctcatgcagacgcagaaggcggccaccaactacgggcgcctgtacgccggcatggtgatcgtcatggtgccggtgctgatcttctacgccgtcgtgcagaagaaactcctcgaaacctcgaccatgggaggaatcaaatgagcgacaggacgaaaagcgtgatcgccacgacggtcaaagtgacgctgtatgtctgtttcgtggtgctgaccatcgtcggcgtcggtcgtgacggatgggacgggctctgcctggaactcatcggattggccggcgtgatcggcgtggtgtggtggtacaacgcgcataaccgatgatccggccggacggtgtgaggcaacggcctggtcacccaggccgttgcctcacaccgtttctcgcccctcccatgaaaaagggcaagaaacgaaggcgcacg

SEQ ID NO: 59 - DNA, 4513 bp, Bifidobacterium infantis
  source 1..4513: mol_type = other DNA
atgagaagaaccgcgatgagggtgggctctgctgtgagcgccgttgccttgctgggttcgctggccgcatgcggcggcgacaagaagccgaccacgaccgccgacggcaagccgatcgtgagcgtcctggttgcgaaggaacccaatcaggacaagatcgccaacatgcagtgggccaaggacctcgaggccgactgcgactgcaagatcgaatggcaggaagtcgataacggagatttcgcgaatcagcgcaataccacgctgagcgccggcgacattccggacatcagcctccatgccttcagcttcgtgtacgcggagcagttctccaacctgttcgaggatctgaaccaggatctcgacaagctgccgaacgtcaaggcgttcttcaaggagaagccggacgcgcagaagctcaataccgattccaagggccacctgtacacgctccccaatgcccacggcaagaacaatgccggcactggccagctcatgatgatcaacaaggcctggcttgacaagctgggtctgcaggtgccgaccacgtgggacgagctggagaacgtgctcaaggcgttcaagacccaggatccgaacggcaacggccaggccgacgagatcccgatgaacatcaagaagctcgacccgttgtactacacctggcacagcccgatgctgctgctcaactccaccggcatcccgaccggcttcaacaagggcgtgtccacatacggcttctacgccaagaacggcgtggtcaagagcttcatgacctccgacgaatacaagcaggtcatcaagtactaccacaagctcatctccgagggcctgatccccgccgactgggccaccaaggacgatgacgcctacaatgcggaccaaaccagcgacggcaagaccgcgaagaccggcgtggtgttcagcagctccatctccgataccttcggcgatctgaaggaccagtacatcaccatcccggtgccgtccgccccgggcgtctcgccggaccagaccgtgtgggacggctcctcgagcgaattcgacgccgccggccccgccgtctccgcgcgcgccgccaacaaggacgccacgttgaagctgctcaacctgatgtactccgagaagtactccgtgcagcagttctacggctccttcggcaagaccgtgaccaagaccggtgagcacgaatacaccgtggacaacgacaagcttatcgagatgcgcaagaagaacacctccccgagcctgtcctaccccgccggctggattcccgacgaggtcaccatcaagggcgatgccggtagcgacgccctgtaccaggcgagcaaggtgtatgagaagcagcgcagcaacttcgatcccgtcaaggactacattccggactacgtgaacccggacgcggacgacaacaccacgttggccagcaactacaaccagatttccaacgtcgccatgcagaagactgctacttggatgtccaagggtggtattgacgaggagtgggatgcgtattgcaagcagctcgacagccttggtctgcaggagaacgtgaagatctggcagaagtggtacgacatctacacgaagaagtgatgtagcggttccgccgttgcatgagcgggtcgtcccgtccgtcgcgcgttcgcgtggcgggcggggcggacgtgggaaggatttttatgtctgtggtgaagcagggtgaagtggtggataacgcgttggcggctcgttccgcgccgttgccggtgcggttggggcgtcatttccgtcggtacggcgcgttgtggctgatgacgttgccggcgttggtgttcgtcggtctgttcgcgtacgtgccgatgtacggtcttcgtctcgcgttctatgatttcgatccggtcgagggtctgatgggcggcaggttcgcggggctgcggtatttcgagcagttcttccggtcgggcatgttcgtgaacatcatggtcaacacgttgcggatcagcctgtggacgctggtcatggggttcatcgccccgatcgtgctggcgttgctgatcaaccagatctcgtcgtcgaagatcaaggggttcgtgcagacggtgacgtacatgccgcacttcatctcgacggtcgtgatcgtgtcgatgatcaacatcttcctgtcgccgagcacgggcatgatcgggcgcctgttcccgggcaccgacctgttgggcgagccggggctgttcacgccgatctactggatcagcgaggtgtggcagcacatgggttggaactgcatcatctatctggcggcgttgagttcggtggacctgtccctgtacgaggccgcgaagatcgacggggccggtcgtctgcagctcatccgctacgtcgacatcccggcgatcatgccgacggtgggcatcatgctgatcatgaacatgggttccgtgctcaacgtgggcttcgagaaggtgctgttgatgcagaacccgatgaacctgtccgtgtcggaggtcatcgcgacgtacacgtaccgcatgggtattctgggcaaccagttcagctacacgacggccataggcctgttcaacaccgtggtcaacttcttcttcctggtgctcgccaacttcatttcgaagagggcgtcggacaccagcatcttctagcgggcgggagggtccattatgacaagcacatccatcaatccggcgccggtccgcggcgcggggtcggacgacatcccgttcaacccgaagctgaggcagaggaggagcgtgtccgactgggtcgtcgacatcgtgatctggctgttgatcgcgttggtcgtggtggcgatcgtctatccgatctggttcatcgtgatcgcctcggtctcggaccagacgatggtcagccagggcaaggtgttcctgttgcccgcgaagatgaacttcggcggttacgccaaggtgttcaccgattcgaggatctgggtcggctaccgcaacaccatcttctattcggtggcgggcacggccctgaacatgctggtcacgatcccggcggcgttcgcgttgtctcgccgcgagttcaagccgcgccgcgtgatcctgttcctgatgacgttcacgatgttcttctcgggcggcatgatcccgagcttcctgctgtacaagcagctgggtctgctgaactcggtgtgggtgttcatccttccgggcgcggtgagcgtgtggaacctgatcgtggcgcgttcgttcttcgagagctcgatcccggagagcctgcatgacgccgcgcagatcgacggcctggggtatttcgggtatttcctgcggatcgtgctgccgttgagctccgcgatcctggcggtgatgacgttgtattacttcgtgggtcattggaacgacttcttcactggcttggtgtacatccgcgacgcggacaagctgccgttgcagaacgtgttgcgttccatcctgttgtcgaaccagacgaacatcaccggtcagagttcgggtggcatggacgtggtgcagcagcgtgatttcgcgaaccagatcaagtacggcgtgatcatcgtgtcgacgttgccgttgctggtgttgtatccgttcctgcagaagtatttcaacaagggcgtgatgatcggtgccgtgaagggctgatcgtcgggggtttcgttgcacgacacacaacgaaacccgcccgtgacgtgtatgccattggcggctccatgagagtggatgccgccaattgttttgtagcgatccatgcggcttcccgcatgaaggaggtaggggcattatgaagcgtctggctgtggggtatgagttcatctgccggatcatcatgatggtggctgtggtgaatgtcgccttcgtgctgcacacgctgcttggctttgtcttgggcgggctgtttccctccgtggccgcgtcgtattcggtgttccgccagtggctgctcgacgtcgaagatcgctcctggaccatcaaacgtacatggacgacgttccatcaggcatggctggaggagatcggcgcggcgaatcagctggggtggccgcagacgatcatctggctgctgctcatctgggaatactggttcgtgcagaacaacgacctgggctatgtggggaccgccgtatccggcgtgctgttggtgctcaacgtgctgtacggcctgttcgtgttcctcagctggtccgtgcacatcaatttcaatgagaatctgctatggacggtgcgcacgtcgctggtcatggtggtcgcacgcccgttgtgcagcttcattgtgctggctcttttcctcgccacggtgtggtcctactacacgtggcccggtctgatgatggcgttcgggccggcggtgccgatcttcttcaccatggtggccgtgtattcgtggggcaaaatgccgggcatggacgtgcgggtgattgagccgttggagaagaaacgatgaggcaaggacgacggattcgacgtcgccagattcattccccgttgtctttcagtattaataaactgattccgcttggcgatacctgatggcaacctgattg

SEQ ID NO: 60 - DNA, 3717 bp, Bifidobacterium infantis
  source 1..3717: mol_type = other DNA
atgagaagaagaaccgcactgaaggcgggcgccatcacgagcgccgtcgccttgctgggttcgctggctgcatgcggcggcagcaaggagccgaccacgaccgccgacggcaagccgatcgtgagcgttctggtcgtgaagaggccggccacggacaagattgccaacatgcagtgggccaaggacctcgaggccgactgcgactgcaagatcgagtggcaggaggtcagcgaggacgcctgggcccagcagaagaacgccaccctggccgcgggcaagattgcggacgtcagcctccatgcgttcttccccgccaacgccgcgcagttcccgggcctgttcgaagatctgagcaaggatctcgacaagatgccgaacgtcaagcagttcttcaaggagaagccggacgcgcagaaactcaccaccgatcccgaaggccacatgtacgccctgccttcgtcccgaggcaagtcctactccggcacgggccagcacatgttcatcaacaagacctggcttgacaagctgggtctgcaggtgccgaccacgtgggacgagctggagaacgtgctcaaggcgttcaagaccgaggacccgaacggcaacggccaggccgacgagatcccgatgaacatcaggaagctcgacagctacttcacctactacagcccgatgctgctgctcaactccaccggcatcgtcaccggcttcaacaagggcgcgtccccgaccggcttctacgccaagaacggcgtggtcaagagcttcctgacctccgacgaatacaagcaggtcatcaagtactaccacaagctcatctccgagggcctgatccccgccgactgggccaccaagacgttcgatgcgtgcgataccgaccagctcagcgacggcaagaccgcgaagaccggcgtgtcgttcggatggtcccaggatgcgagcttcggaacactcaaggatcagtacatccccatcccggtgccgtccgccccgggcgtctccccggacaagaccgtgtgggacggctcttccgccgagttcgaagccgataggttctccctgtcctcccacgccgcgaacaaggacgccgcgctgaagctggccaacctgctctactccgagaagtactccgtgcagcagttcctcggctccttcggcaacctggtcaccgatgacggcaaccgccactacaccgtggacgaagacaagtacaccaaggcgatgggcgacaacctcttcccgggcttggccgatcgtttctccggctggattcccgatggcgtcaccatcaagggtgatgtcgacggcgacaacctgcttgaggcgaacaagccgtacgaggagcagcgcagccacttcgatcccgtcaaggactacattccggactacgtgaaccccgatccgacggacagtaatacactgaccaataacaatgcacagatttccaatgttgttatgcagaagactgctacttggatgtccaagggtggtattgacgaggagtgggatgcgtattgcaagcagctcgacagccttggtctgcaggagaacgtgaagatctggcagaagtggtacgacatctacacgaagaagtgatgtagcggttccgccgttgcatgagcgggtcgtcccgtccgtcgcgcgttcgcgtggcgggcggggcggacgtgggaaggatttttatgtctgtggtgaagcagggtgaagtggtggataacgcgttggcggctcgttccgcgccgttgccggtgcggttggggcgtcatttccgtcggtacggcgcgttgtggctgatgacgttgccggcgttggtgttcgtcggtctgttcgcgtacgtgccgatgtacggtcttcgtctcgcgttctatgatttcgatccggtcgagggtctgatgggcggcaggttcgcggggctgcggtatttcgagcagttcttccggtcgggcatgttcgtgaacatcatggtcaacacgttgcggatcagcctgtggacgctggtcatggggttcatcgccccgatcgtgctggcgttgctgatcaaccagatctcgtcgtcgaagatcaaggggttcgtgcagacggtgacgtacatgccgcacttcatctcgacggtcgtgatcgtgtcgatgatcaacatcttcctgtcgccgagcacgggcatgatcgggcgcctgttcccgggcaccgacctgttgggcgagccggggctgttcacgccgatctactggatcagcgaggtgtggcagcacatgggttggaactgcatcatctatctggcggcgttgagttcggtggacctgtccctgtacgaggccgcgaagatcgacggggccggtcgtctgcagctcatccgctacgtcgacatcccggcgatcatgccgacggtgggcatcatgctgatcatgaacatgggttccgtgctcaacgtgggcttcgagaaggtgctgttgatgcagaacccgatgaacctgtccgtgtcggaggtcatcgcgacgtacacgtaccgcatgggtattctgggcaaccagttcagctacacgacggccataggcctgttcaacaccgtggtcaacttcttcttcctggtgctcgccaacttcatttcgaagagggcgtcggacaccagcatcttctagcgggcgggagggtccattatgacaagcacatccatcaatccggcgccggtccgcggcgcggggtcggacgacatcccgttcaacccgaagctgaggcagaggaggagcgtgtccgactgggtcgtcgacatcgtgatctggctgttgatcgcgttggtcgtggtggcgatcgtctatccgatctggttcatcgtgatcgcctcggtctcggaccagacgatggtcagccagggcaaggtgttcctgttgcccgcgaagatgaacttcggcggttacgccaaggtgttcaccgattcgaggatctgggtcggctaccgcaacaccatcttctattcggtggcgggcacggccctgaacatgctggtcacgatcccggcggcgttcgcgttgtctcgccgcgagttcaagccgcgccgcgtgatcctgttcctgatgacgttcacgatgttcttctcgggcggcatgatcccgagcttcctgctgtacaagcagctgggtctgctgaactcggtgtgggtgttcatccttccgggcgcggtgagcgtgtggaacctgatcgtggcgcgttcgttcttcgagagctcgatcccggagagcctgcatgacgccgcgcagatcgacggcctggggtatttcgggtatttcctgcggatcgtgctgccgttgagctccgcgatcctggcggtgatgacgttgtattacttcgtgggtcattggaacgacttcttcactggcttggtgtacatccgcgacgcggacaagctgccgttgcagaacgtgttgcgttccatcctgttgtcgaaccagacgaacatcaccggtcagagttcgggtggcatggacgtggtgcagcagcgtgatttcgcgaaccagatcaagtacggcgtgatcatcgtgtcgacgttgccgttgctggtgttgtatccgttcctgcagaagtatttcaacaagggcgtgatgatcggtgccgtgaagggctgatcgtcgggggtttcgttgcacgacacacaacgaaacccctttgttcattcggccgtcgcgcgcttccccggtcgcccgtcgtggtgccacatattgttag

SEQ ID NO: 61 - DNA, 3400 bp, synthetic construct
  misc_feature 1..3400: note = Codon optimized sequence encoding TP10
  source 1..3400: mol_type = other DNA
atgaagaaaaaggtgattagcctgatgctgattggtgcaatggttgcaagcatggcagcaagcggtagcaatggtggtgataaaggtggcaccaaagataatgataccagcgcagcagcagaaagtgatggtggtgcaagcgaaggtggcgataccctgaccgtttggacctgggatccgaaatttaacattccggcaattaaagaagcagccaacatctataaagcagatcacccggattttaacctgaaagttgttgaaaccctgtccgatgattgtgaaagcaaactgattgcagcagccgaaggtggtgatctgagcaccctgccggatattgttctgatgcaggataattcctatcagaaatttgttaccgcatatccggaagcctttgaagatctgaccgatagcggtattgatttttcacagtttccggcaggtaaactggcatatagcaccgttgatggtaaaaactatggtgtgccgtttgataatggtgcagttattggtgcatatcgcaccgatattctggaacaggcaggttataccaccgcagatttaaccgatattgattggaaccgctttatcgagattggcaaagatgttaaagcaaaaaccggcacctatatgctgagcggtcaggcagatagtccggataccattatgatgatgctgcagagtgccggtgcaagcctgtttgatgaagatggtaaaccggcaatgaccgataatgcagcactgaaagaatgcatcgatatctacaaaaccatggtggatgaaggcatttatctggaagtgaacaaatgggatgattacgttaccagcattaccaaaggtagcgttgccggtgttattaatggcaattggattattgccaccatccaaggtatggaagataccgcaggtaaatgggaaattaccaatatgccgaaactgatcaaaaccccgaatgcaaccaattatagcaataatggtggtagcagctggtacattacctccaattgtaaaaacaaagatctggcgatcgacttcctgaaaagcacctttgcaggtagcgtggaactgtatgataaaattctggcaagtaccggtgcaattgccacctatctgccagccggtgaaagcgaaaaatatgcagaaccggttgcctattggagcgatcagccgatctataaaacaatcgttgattacagtaaactgacccctgccaataataccagtccgtattattacgatggtcgtaatgcaattggcacccagattcagaatattatgaacggtgcagataccgattcagcaattgcagatgcacaggccgaagttgaatttagcatgcagtaattcatttctgattttgagatgacaacctatttttaaaaatatgcggggggctgcctggcagttccccgaatattatttgcgaagggggttttgaggtggaaccgaaaaagaaaggtatgaccctggttgcaaaacagaaagcagcaggttggctgtttctggttccggcaagcgcaatgattgcaattatgagctttttcccgatggtgaaagcctttattatcagcctgcagaccggtcgtggtctgaatatgcattttgcaggttttagcaattacgtgcgcatgtttcaggataaagtttttctgcgtagcgtgggcaataccttcttttatctgattattcaggtgccgattatgctggttctggcaattctgctggcacagctgctgaataaccgtgatctgaaactgcgtggtttctttcgtacctgtgtgtttctgccgtgtgcaaccagcctggttagctatgcactgatttttcgttttctgtttgcccaggatggctttattaacaatgccctgattggtctgggtattctggataccggttataactttctgggtaatgccatgagcgcaaaattcgttattatcattgcactggtttggcgttggaccggctataatatggtgttttatctggcaggtctgcagaacattgaatatagcgtttatgaagccgcaaaaatcgatggtgcaaatggctggaaaaccttttggagcattaccgttccgctgctgaaaccgaccattattatgacgtttatcatgagcattaacggtacgctgcagctgttcgatgaaagcgttaatctgaccaatggcggtccggcaaatgccaccattaccatgagccattatatctataacagctgctttgttcgcagcccgaattttggttatgcaagcgccatgagtttcttcgtatttatccttgtggcaatccttgcgtttatcaatatgaaagtaggtgatacacgtgactaaaaagaaaaacaaatcaatgattcagcgtcgtctgattcctacttatattttcctggcaattgttagcctggtgagcgtttttccgttttattggatgattatggcagccaccaatgactatgttgatattagccgtggcaaactgacctttggcaccgcagcactggaaaactttcgtaatctgattgatcagcagcctctggcactggcaatgaaaaattcatttgtttatgccattagcgtgaccgttatttgtctgctgatttgtagtctggcaggctatggctatgaaatctatcacgataaatggaaagacaaactgttcagcgttatcctgctgggtatgatgattccgcaggttgcgaccattgttccgctgtatcgtatgtttaccaaagcgcatctgattaataccgcagttggctttttcctgccgatgatcagcaccccgtttatgattatgatgtttcgtcagaatagccgtgcatttccggttgatattgttgaagcagcacgtattgatggtctgagcgaaatcaaaatcttctttcagatgtatatgccggtgatgaaatcaacctatgcagccgcagcagttattacctttatgaatgcatggaatgcatacctgtggccgaaagtgattatgaccgatggcgattcactgaatatggttatgctgatcgcaaatatgaccggtggttataaactggattacgggatgatcatgatgggtgtgctgttttgtagcattccgacaatggtagtcttctttgtactccagaaacagtttgcagaaggtattacaggtgccgttaaataatgacgagataaatagtcatttcaaacataatattgcaacttatgcaacccctcgttggtttcgtcgtggtgtttttcagaccgaacagaatcgtaaatgttatagcgtgctgattaacaaaaaggtgctgtatggtattcgcacctaataagttcggaaaggaaatgaaaaatgggcgtgaaatttgatcgtttatgttacggtggagattataatccggatcaatggctggagtgtccggagattc

SEQ ID NO: 62 - DNA, 3473 bp, synthetic construct
  misc_feature 1..3473: note = Codon optimized sequence encoding TP17
  source 1..3473: mol_type = other DNA
atgaccaatgcaaccgcaaaaccggataccagcggtatgcgtaaaccgaaacgtgaatatattggcattctgtattgtctgccgtatgttgtggtttttctgtttggtatgatcatcccgatgttctatgcactgtatctgagctttttcaaacagagcctgttaggtggcaccacctttgcaggttttgataattttctggcagcctttaaagatgaagcactgtggtcaggttttcgtcgtgttctgatttatgcagcaattcagattccgctgaacctgattctgagcctggcagcagcactgattctggatagccagcgtattcgtcatattgcagttccgcgtattctgctgtttctgccttatgccgttccgggtgttattgcagccctgatgtggggttatatctatggtgataaatatggtctggttggtcagattgcaagcatgattggtgcgaccgctccgaatatgctgagcgaaaaactgatgctgtttgccattgccaatatttgtacctggtgtagcctgggctataacatgctgatctattatagcgcactgattggtattccgaacgaactgtatgaaagcgcacgtattgatggtgcaagcgaactgcgtattgcatggtcagttaaaattccgcagattaaaggcagcattatgatgaccgttctgtttagcgttattggcaccctgcagctgtttaatgaaccgagcattctgatgaatagcgcaccgaatgttattaccagcaattataccccgaatctgtatgcatataacctggcatttaatggccagaatctgaactatgcagcagcagttagcctggttattggtgtgattgttatgggtcttgtcgcagttgtgaaggtcattggcaataagtgggagaacaagtgatgagtgaagcaagtgcaaaaccacgttccaagtcgctgcagcgcagagacgcaaaactggcactgaaagcaagccagcgttataaacgtatgcaacagcgtgaagcagcaccgaaactgaccggtaaacagcgtgtgctgaattggctgctgcacattattatggcaatgatggtgatttattgtctggttccgctgctgtgggttttctttagcgcaaccaaaaccaatgaaggtatgtttagcagctttggcctgtggtttgatgataaaaatgtgttttggcagaacgtgcaagaaaccctggcatatcagcatggcacctatgcacgctggctgctgaataccgttatgtatgcagttgttgccggttttggtgcaaccattattgcgaccctggcaggttatgcaattgccaccatgcgttttccgggtcgtaatagcctgctggcaattaccctggcctttatgagcattccggcaaccgttattagcgttcctctgtttctgatgtacagcaaaattggcctggttggcaccccgtgggcagttattattccgcagctggcaaccccgtttggtctgtacctgatgattatctatgcacagaccagcgttccgaccagtctgctggaagcagccaaactggatggtgcgaatagctggaccatcttttggaaagttgcatttcctctgctgagtccgggttttgttaccgtgctgctgtttacactggttggtgtttggaataactattttctgccgctgattatgctgtccgataccaatgattatccgatgaccgtgggtctgaatatgtggctgaaaatgggtgccgaaggcaccgcagatggccagattccgaataattacattgttaccggttgtctgattgcagttgcaccgctgatcattgcatttatgtttctgcaaaaatactggcagagcggtctgagtgccggtgcagttaaacagtaaaccctcgccgcacggttattgacgatcacccattcgttgctctaatgaccgtgcggcatccacacccatttaaagaataacaataagacagtaattcatgacacacaaaggagtaatcatgaagaaaatgacccgtattgtgagcgcactggcagcgctgaccatggttgcaagcgttgcagcaagcggtagcagtaccggtgcaaaagataccaaaacagcagttaccgtggaagatattaactcagcactgaccgataccagcaaaaaggttgaactgaatgtttggggttttagcggtaaacaaatggcaggtccgattgaagcattccagaaaaagtatccgcacatcaaaatcaagtttaccaataccggtgccgcaagcgatcattttaccaaatttcagaatgttgtggcagccaacaaagatgttccggatgttgttcagatgagcgcagatcgttttgaacagtatgcaattagcggtgccctgctgaattttgcgaatgatgatatcgaaaaagcctggtcaaaactgtatgcaaaaaccgcatggtcacaggttcattatgccggtggtctgtggggtgttccgaccgatgcaacaccgctggcaatgtatgttcgtaaagatattctggatgagcacaatctgaaagtgccgaccacctggcaagaattttatgaagaaggtgtgaaactgcacaaagaggatccgagcaaatatatgggtgtgctgattaacaatgacatcagctgttttaccgatctgctgcgttcagccggtgcacgtccgtggaaagttaatagcgttgatgatattagcctgagcatgaccagcggcaccaccaaagaatttattgaattcctgcagaaatgcctgcaggatggtgttatggaagccgcacctggttggaccgatgcgtttaatcgtgcaacaaatgaaggcacatatgccacctatatttcacagaattggcagggcaatagctacaaaggtcagaatccgagcctgaaaggtctgattcaggttaccctgcctccggcatttggtgatagcgcagatgatttcaaaagcagcagcgttggtagcatgatgagcgttagcgcagcatgtccgaaagataaacaggcagcagccctggcgtttattaactggctggatagcgataaagaagccattcagagctggcaggataataatcatggcaactattttatggcagcgagcgcatatcaggatagcgaagatattcgtaatatgaaagaaaccgatggctacttcgttaataccgatgtgaatagcgtgtatttcgagagcatggataaagtgaataccgattgggaatatctgccgtttatgagccaggttggtgtggtttataaagatgtgattacccctgaaatgaaagtcggtggtgatatggttggtgcaatggccaaagcacagcagaaactgaaaacctatgccaaagataacggctttaaagttaccaccgatgccgattaacatccggttgcccgccactgctagggagggcggccgaatggccgtcctccctttttgagattaaacatttttcc

SEQ ID NO: 63 - DNA, 1443 bp, synthetic construct
  misc_feature 1..1443: note = Codon optimized sequence encoding TP4
  source 1..1443: mol_type = other DNA
atgagccagcagacccagagccagattgatggtaaactgaaaggcaaactgattgccgtttgtaccattggtgcagttggtggttttctgtttggttatgataccagcgttattaatggtgccgttaccgcaattgcaagtccggatagcggttttgcactgagcagcaccatgagcggttttagcgttagcagcgcactgctgggttgtgtttttggtgcatggtttgccggtgttgttgcagatcgtatgggtcgtattcgtgttctggaactggcagcaattctgtttgttgcagcaagcctggttaccggtctgaccaccaattttaccatctttatcatctttcgcatcgttggtggcttaggtgttggttttaccagcgcaattggtccggcatatatcagcgaaatttcaccggcagcacgtcgtggtttctttaccagctttcagcagatggcaattgcactgggtattattgccagcctggttgtgaatgatatttatgttgttgccagcggtggtgcagaaatgccgttttggatgggtattgatgcatggcgttggatgctggttaccaccgcactgcctggtgtgattatgtttttcctgtgttttgccctgccggaaacaccgcgttatctggttatgaaaggtgaaattgcaaaagccaaaaccatcctgattgatgtgattggcgataataatgccgatgtgaccattgccaaaatccagaaaagctttcagagcgaagaaaacgaaaagaaagttagctttcgtgaactgcgtggcaaaacctttggtctgaaaggtgttgtttggatcagcattatctttgttctgctggcacaggcaaatggccagaacattattatgctgtatgatagcagcctgtggaatatgctgggttttagtgaacaggcaagcctgacaattgcagttgttcgtgcaaccctggcagcagttgcaaccgttgttggtatggttctgattgatcgcgttggtcgtcgtaatctgatgaaatatggcaccctgctgatgaccgtttttctgggcattaccgccattggttttggtcagggtcagctgagcgcagatggtacactgagcctgagcattacctggacctggattagcattgtttgtacctatgcattctttctggtttgttgtgccaccaccaatgtgaccgtttgggtgattattagtgaaatttttccgaatcgcattcgtgcaaaaggtgaagccgttgccaccgcagcaaattggatgtttaactttctgatcaccaccatctatccgcctatgcgtgatggtattggtttaggttggacctatggtttttataccctgatggccctgctgggctttttctttgttgttaaatttctgcctgaaaccaacggtgttgaactggaagatatgaaagccgaactgaaataa

SEQ ID NO: 64 - DNA, 1218 bp, synthetic construct
  misc_feature 1..1218: note = Codon optimized sequence encoding TP1
  source 1..1218: mol_type = other DNA
atggcactggatgttggtaaagcactgaaaagcaaaaccctggttgttggtgttctgagcatgagcctgctgatgagcgcaagcaatgcagttagcggcaccattccggcaatgaaagaagcatttagcgattatagcgcagccaatgttgaactgctgaccaccgttccgaccattggtagcatggttggcaccgcactgaccggtctgtttgcaaatgcaattggtcgtaaaaagattgcaatggcaggttttctgattagcgcagttaccggtgttattccggcatttttcccgtattattggccgattctgatttcccgcatgtttttcggttttggtagcgcactgtttgttaccctgagcgttagctatatcaccgatctgtatgatggtgatatgcagcgtaaactgttaggttggcgtcaggcagttggtaatctgggtgatgttgttctgctgtttgttgcaagtctgctgattaccattaattggcagagcacctacctgattttctttctgttatttgttccgatggttctggtgggtatgtttatcccgaaagaatttgataactttagcattcgcagcgcactggttgatgatgaaggtcatgttgttgataaaagcgcaagccagaaacagaccaccaattggcaggttctgtggctggcatttatctttctggttgttagcatgctgtataacgtgatgagcattaaactggccagctatgttgtggatgaaggtattggtagtgcaagcctggcaaccctgatttttagttttctggtggttgccaccattctgagcggtgttctgtttgataaagttgccaaagttaccaaacgtctgaccgttaccattagcgaagttgttattggcatttgctttattgcaacagccctgaccaaaaatgttccgctgatgtttgcactggttctgattgcaggttttgcatggggtattatcaatccggcactgacagcacgttttgttgattatagtccggcacatagcatgaatctgaccaccagtattgtgattatcggcattaacattggttgcctgattagcccgtatttctttgccctgaccgcaagtatttttggtaatagcagtgccggttttgccattattgttggtggtgcactgtatctggttatggttgtgattgaactgattaccctgaaagtggataaaaagctgaccgtgtaa

SEQ ID NO: 65 - DNA, 2220 bp, synthetic construct
  misc_feature 1..2220: note = Codon optimized sequence encoding TP2
  source 1..2220: mol_type = other DNA
atggcaggtctgtttcgtaccgcaagcaaacagccgaatccgctgggcaccgcaattcgtacccgtggtagcggtaaatatacctttggtctggcactgagtgccggtctggctggtctgctgtatggttatgataccgttagcattagcggtgccattgaatttctgcgtgcacgttatggtctgagcaccctgatggaaggtctggttattagcagcattatgctgggtgcagttattggtgcagcaaccgcaggttttctgagcgatcgttttggtcgtaaacgcattctgattattggtggtgcatttttcctggttgcagcagtttggagcgcactgaccattggtccgattgcactgattgttgcacgtgttgccggtggttatggtattggtctgaccgcagcactggcagttacctatatcaccgaaagcgcaccggcaaatattcgcggtctgctggcatttagctatcagctgctggcagtttgtggcctgtttctgaccaatgttatcaactatatcattgccagctatggcagcaacgattgggatattgcaattggttggcgttggatgttaggtctgggtgcaattccggcagcagcatttctgctggccatgcgtcgtgcaccggaaagtccgcgttttctgattcaggttggtcgtaccgatgaaggttttgcagttctggaacatattctgggtacagaacgtgcccgtctgcgcaccgatgatattcaggcaagcgttaaactggaaaccgaaatgagccatgaatttcatgacctgtttcgtccgggtctgcgtcgcgcactggtgattggtatttttctggccgtttttaaccagttcattggcatgaatgccatcagctattatggtccggttatgtttagcgatttaggttttggtggtgatacccagtttctggcagcagccagcgttggtggtgttgaactggtggcaaccgttgttggtatgtatctgattgatacatttggacgcaaacggctgatggaaattggcaccggtatgatgtgtgcatttgcactgtgtattagcggcagctattttatgggtaatagcctgctgaccctgatttttgttatggcatttaccattagcttcgcctttagcatgggtccgattccgtggattgttattccggaactgtttccgacctatctgcgtggtcgtgcaaccggtctgtgtgttatgtgtctgctgtttgcaaattggattattgcacagtttaccccgatgatgattgatggtttaggtggtggtattagctttgccatttttgcctttctggatctgatttgcctgtttggtattgttgccctggttccggaaaccatgggtcgtaccctggaagaaattgaacatctgtggcagccgaaaaccgatctggcaagcgcaaaatatagcctgagcaccgcagatgccaatattcgtcatgcagaagcaaccctgcgtcgtattgaaaatgaacgccagcaggcactgggtattatggatgcagccgaacgtgcgcgtgcagcagcacagcagaaaatctttgcaattgaaaccgcaaaacgtgccgaagcagaacgcctgaccgcacagcgtgccgcagaagaagcagcaaaagcagcagcaaatatggcagcggaagaagataccgcagccaccgttagcgaagttaccaccgatgaaaatgttccgagcctggttcgtcgcacccgtggtgccgttctggttggtaaaagcagctcacgtcgtcatgatggtgaatataccggtggtccggcagatggtagcgttaccggtagcgcagccggtgcaatgctggatggtgcagcggcagaagcagccgcaagcgcagcagttgcagccctggcagatagcctgccggatgatccgtgtacactggcaaatcgtccgaccacacgtagcggtgcagatgataccgatagtgttgatgcagcagatgcagttgattttgcaaatgatgcaaatcgcaatcgcaaacagaccaatgcagttcagcctgcacatatggcaagcattgaacatgcacatagcgcagatacaccgcgtggggttctgctgccgagcgcagatgaagatagctatagcgaacatagctttgcagaaacggatgatgatcgtgaagcacaggcaattgatgccgcactggatagcctggatgccctgattcgtggttaa

SEQ ID NO: 66 - DNA, 1326 bp, synthetic construct
  misc_feature 1..1326: note = Codon optimized sequence encoding TP3
  source 1..1326: mol_type = other DNA
atggcaagcgcaaccaaaagcgcatggaaaaatccgagctatctgcagagcagctttggcatttttatgtttttctgtagctggggcatttggtggtcatttttcagccgttggctgaccgatccgacacatggtctgggtatgaccagcgcagaacagggtcagatttatagcattaatagcctggcaaccctggtgattatgtttgcatatggtgcaattcaggatcagctgggtattaaacgtaaactggttatttttgttagcgccattgcagcactggttggtccgtttgttcagtttgtttatgcaccgatgctgaccgcaggcggtacaacccgttttattggtgttctgattggtagcattgttctgagcagcggttttatggcaggttgtagcctgtttgaagcactgaccgaacgttatagccgcaaatttggttttgaatatggtcagagccgtgcatggggtagctttggttatgcaattgttgcactgtgtgcaggctttctgtttaacattaatccgctgctgaatttttgggtgggtagcatttgtggtctgggcatgctgtgtgtttatgccttttgggttcctgccaaacaaaaagaggaactgaagaaagaagcagatccgaacgcagcaccgaccaatccgagttttaaagaaatgattagcgtgctgaaaatgccgacactgtgggtgctgattgtttttatgctgtttaccaacaccttctacaccgtttttgatcagcagatgttcccgaactattatgcaagcctgtttccgaccaccgaaattggtaatgcaacctatggcaccctgaatagttttcaggtttttctggaaagcgcaatgatgggtgttgttccgattatcatgaaaaagattggtgtgcgtaatagtctgctgctgggtgcaaccgttatgtttctgcgtattggtctgtgtggtgtgtttcatgatccggttagcgttagcattgtgaaactgtttcatagcattgaagttccgctgttttgtctgcctgcatttcgttattttaccctgcactttgataccaaactgagcgcaaccctgtatatggttggttttcagattgcaagccagattggtcaggtgatttttagcaccccgatgggtgcactgcatgatgcaatgggtgatcgtccgacctttttcaccattagcggtattgtgctggcagcactgatttatggtttcttcgtgatcaagaaagacgatcaagaggttggtggtgatccgttttataccgataaacagctgaaagcacaggcagcagcaaaagcaaatgcataa

SEQ ID NO: 67 - DNA, 1227 bp, synthetic construct
  misc_feature 1..1227: note = Codon optimized sequence encoding TP13
  source 1..1227: mol_type = other DNA
atggcaattctgctgctggccgttatttatgttgcatttattagcctgggtctgccggatagcctgctgggtagcgcatggccgaccatgagccagaatctgaatgttccggttgcatgggcaggcggtattagcgcagttattagcatgtttaccattgttagcgcactgctgagcgatcgtatgaccctgaaatttggtgcaggtaaagttaccgcagttagcgttgcactgaccgcagcagcactggcaggttttagcattgcaccgaattattgggttctgctggccattgcaattccgtatggtttaggtgccggtggtgttgatgcagcactgaataactatgttgccatccattatgaaagccgtcatatgagctggctgcattgtatgtggggtgttggtgcaagcgttggtccgtatattatgggttatgcactgagccaaggtcaaggttggccgtggggttatcgttatattgccattctgcaggttatgctgaccgtgattctggttctgagcctgccgctgtggaaaaagcgtggtacaaccggtggtggtgaaagcaccgatggtgcagcaagcagtgatggtaatgccgaaggttgtggcaatgcagaaggtgccagcgttgcagaacgtaaaccgctgggtgttgccggtgttctggcaattcgtggtgcaaaagaaattctggttatgttcttttgctattgcgccattgaaagtaccgcaggtctgtgggcaagcagctatatggttatgcatagcggtattgataaaatcaccgcagcaagctgggcaggcctgttttatgttggtattaccgttggtcgtgccctgagcggttttctgagcatgcgttttaaagatccggtgatgattcgtctgggtcaagttctggtgctggcaggtattctgaccatgtttgttccgctgccgcatcatctgggcgttgttgttggtctggttgttattggttttggttgtgcaccgatttatccgtgtgttattcatagcacaccggcatattttggcgaagataaaagccaggcagttgttggtgttcagatggcatgtgcatatgttggttcactgctgatgcctccgctgtttggtattattgcacagtatgttaccattagcctgtatccgtggtatctgctggtgctgctggtactgatggttgcaatgcatgaacgtctgcgtaaactgcgtggttaa

SEQ ID NO: 68 - DNA, 1284 bp, synthetic construct
  misc_feature 1..1284: note = Codon optimized sequence encoding TP14
  source 1..1284: mol_type = other DNA
atggcaagcaataccccgaccaccaccagtaaaagcacccgtggtaaaagctggattattgcatttgcactggttaccctgctgtttttcacctggggtctgaccatgaatctggttagcgcaattcgtgatccgtttgcagtttatctgaatctgaatagcaccgaaagcagcctgctgcaggttgcatattttggtgcctattttgttatggcaattccggcaaccatggtggccaaacgttttggttataaaggtggtatgatttgcggtctgatctttttcgttattggtagctttctgaccattcctgcaaccaccagtctgagctatggtctgtttctgctggcaatgtttgttattgcactgggtgcagcaagcctggaagcaaattgtaatccgtatattatcaaactgggcgacgaaaaaggtgaaagttttcgcctgaatctggcacagagctttaatgcagttggtagcgttgttagtccgctgattctgggtatgatcctgggtaataccgcaagcgcaaaaccgggtgatccgggttttgaagaaagcaaagttgcatttatgagtcagacccgcatgttctatattgtgattggtgttattctggccattgtgatcgccgtttttgtgattgcaaaactgccgagtccgcctggtgatgaagaagaagcagcaggcggagttgcacaggatagcagcattggtgccctggttaaaaagccgtgggttgttctgggtgttattgccgaatttgtttatgttggcctgcagaccattggctttagcacctttagtacctttgcacagcagcagtgggaaggtatgagcgcaggtatggcagcaacctttctggcagttctgaccctgatgtataccattggtcgttttgttaccacaccgtttatggcaaaatttgaaccgggtaaaatcctgggcttttatatgattggtgcaaccattctgatgattctgaccgcaatgggtctgggcaaaattagcgttattacctttattgccctgtatttcttcatgagcattgcctatggcaccatttttgccctggcactgacccatctgaaaggtgcagcagcaaaaaccggtagcagcctgattaccatgagtattgttggtggtgcaattattccgctggtttatggtgcgattaccgatgcagcaggtatgaatgttatgctgtgggcaaccgttccgctgtttctgttcctggtttggtatgcattttggggtagcaaaattggtctgaaagaagcctaa

SEQ ID NO: 69 - DNA, 3472 bp, Bifidobacterium infantis; identical to SEQ ID NO: 51
  source 1..3472: mol_type = other DNA

SEQ ID NO: 70 - DNA, 300 bp, synthetic construct
  source 1..300: mol_type = other DNA
gcggcacgccttgcagattacggtttgccacacttttcatccttctcctggtgacataatccacatcaatcgaaaatgttaataaatttgttgcgcgaatgatctaacaaacatgcatcatgtacaatcagatggaataaatggcgcgataacgctcattttatgacgaggcacacacattttaagttcgatatttctcgtttttgctcgttaacgataagtttacagcatgcctacaagcatcgtggaggtccgtgactttcacgcatacaacaaacattaaccaaggaggaaacagct

SEQ ID NO: 71 - DNA, 185 bp, Escherichia coli
  source 1..185: mol_type = other DNA
tgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctgggtacctaaggaggaaacagct

SEQ ID NO: 72 - DNA, 185 bp, synthetic construct
  source 1..185: mol_type = other DNA
tgtgagttagctcactcattaggcaccccgagctgttgacaattaatcatcggctcgtataatgtgtggaattgtgagcggataacaatttcacacaggaaacagctatgaccatgattacgccaagcgcgcaattaaccctcactaaagggaacaaaagctgggtacctaaggaggaaacagct

SEQ ID NO: 73 - DNA, 185 bp, synthetic construct
  source 1..185: mol_type = other DNA
tgtgagttagctcactcattaggcacccgagctgttgacaattaatcatcgaactagtttaatgtgtggaattgtgagcggataacaatttcacacaggaaacagctatgaccatgattacgccaagcgcgcaattaaccctcactaaagggaacaaaagctgggtacctaaggaggaaacagct

SEQ ID NO: 74 - DNA, 446 bp, synthetic construct
  source 1..446: mol_type = other DNA
ctggcacaggaacgttatccggacgttcagttccaccagacccgcgagcattaattcttgcctccagggcgcggtagccgctgcgccctgtcaatttcccttccttattagccgcttacggaatgttcttaaaacattcacttttgcttatgttttcgctgatatcccgagcggtttcaaaattgtgatctatatttaacaaagtgatgacatttctgacggcgttaaataccgttcaatgcgtagatatcagtatctaaagccgtcgattgtcattctaccgatattaataactgattcagaggctgtaatggtcgttattcatcactcatcgcttttgtgatggcgaccattgacttctgtagagggtgaagtctctccctattcagcaatgcaacctcgtgttgccaggctcaaattacgagcaaaccaaggaggaaacagct

SEQ ID NO: 75 - DNA, 130 bp, Escherichia coli
  source 1..130: mol_type = other DNA
acattgattatttgcacggcgtcacactttgctatgccatagcatttttatccataagattagcggatcctacctgacgctttttatcgcaactctctactgtttctccataccgtttttttgggctagc

SEQ ID NO: 76 - DNA, 1254 bp, synthetic construct
  source 1..1254: mol_type = other DNA
atgtactatttaaaaaacacaaacttttggatgttcggtttattctttttcttttacttttttatcatgggagcctacttcccgtttttcccgatttggctacatgacatcaaccatatcagcaaaagtgatacgggtattatttttgccgctatttctctgttctcgctattattccaaccgctgtttggtctgctttctgacaaactcgggctgcgcaaatacctgctgtggattattaccggcatgttagtgatgtttgcgccgttctttatttttatcttcgggccactgttacaatacaacattttagtaggatcgattgttggtggtatttatctaggcttttgttttaacgccggtgcgccagcagtagaggcatttattgagaaagtcagccgtcgcagtaatttcgaatttggtcgcgcgcggatgtttggctgtgttggctgggcgctgtgtgcctcgattgtcggcatcatgttcaccatcaataatcagtttgttttctggctgggctctggctgtgcactcatcctcgccgttttactctttttcgccaaaacggatgcgccctcttctgccacggttgccaatgcggtaggtgccaaccattcggcatttagccttaagctggcactggaactgttcagacagccaaaactgtggtttttgtcactgtatgttattggcgtttcctgcacccatgatgtttttgaccaacagtttgctaatttctttacttcgttctttgctaccggtgaacagggtacgcgggtatttggctacgtaacgacaatgggcgaattacttaacgcctcgattatgttctttgcgccactgatcattaatcgcatcggtgggaaaaacgccctgctgctggctggcactattatgtctgtacgtattattggctcatcgttcgccacctcagcgctggaagtggttattctgaaaacgctgcatatgtttgaagtaccgttcctgctggtgggctgctttaaatatattaccagccagtttgaagtgcgtttttcagcgacgatttatctggtctgtttctgcttctttaagcaactggcgatgatttttatgtctgtactggcgggcaatatgtatgaaagcatcggtttccagggcgcttatctggtgctgggtctggtggcgctgggcttcaccttaatttccgtgttcacgcttagcggccccggcccgctttccctgctgcgtcgtcaggtgaatgaagtcgcttaa

SEQ ID NO: 77 - DNA, 1254 bp, synthetic construct
  source 1..1254: mol_type = other DNA
atgtactatttaaaaaacacaaacttttggatgttcggtttattctttttcttttacttttttatcatgggagcctacttcccgtttttcccgatttggctacatgacatcaaccatatcagcaaaagtgatacgggtattatttttgccgctatttctctgttctcgctattattccaaccgctgtttggtctgctttctgacaaactcgggctgcgcaaatacctgctgtggattattaccggcatgttagtgatgtttgcgccgttctttatttttatcttcgggccactgttacaatacaacattttagtaggatcgattgttggtggtatttatctaggcttttgttttaacgccggtgcgccagcagtagaggcatttattgagaaagtcagccgtcgcagtaatttcgaatttggtcgcgcgcggatgtttggctgtgttggctgggcgctgtgtgcctcgattgtcggcatcatgttcaccatcaataatcagtttgttttctggctgggctctggctgtgttctcatcctcgccgttttactctttttcgccaaaacggatgcgccctcttctgccacggttgccaatgcggtaggtgccaaccattcggcatttagccttaagctggcactggaactgttcagacagccaaaactgtggtttttgtcactgtatgttattggcgtttcctgcacctacgatgtttttgaccaacagtttgctaatttctttacttcgttctttgctaccggtgaacagggtacgcgggtatttggctacgtaacgacaatgggcgaattacttaacgcctcgattatgttctttgcgccactgatcattaatcgcatcggtgggaaaaacgccctgctgctggctggcactattatgtctgtacgtattattggcacctcgttcgccacctcagcgctggaagtggttattctgaaaacgctgcatatgtttgaagtaccgttcctgctggtgggctgctttaaatatattaccagccagtttgaagtgcgtttttcagcgacgatttatctggtctgtttctgcttctttaagcaactggcgatgatttttatgtctgtactggcgggcaatatgtatgaaagcatcggtttccagggcgcttatctggtgctgggtctggtggcgctgggcttcaccttaatttccgtgttcacgcttagcggccccggcccgctttccctgctgcgtcgtcaggtgaatgaagtcgcttaa

(19) United States
(12) Patent Application Publication
(10) Pub. No.: US 2025/0382649 A1
(43) Pub. Date: Dec. 18, 2025

(54) CELLS WITH TRI-, TETRA- OR PENTASACCHARIDE IMPORTERS USEFUL IN OLIGOSACCHARIDE PRODUCTION

(71) Applicant: DSM IP ASSETS B.V., HEERLEN (NL)

(72) Inventor: Manos PAPADAKIS, Hørsholm (DK)

(21) Appl. No.: 18/860,937

(22) PCT Filed: Dec. 1, 2022
(86) PCT No.: PCT/EP22/84101

§ 371 Date: Oct. 28, 2024

(30) Foreign Application Priority Data
Dec. 1, 2021 (DK) PA202170594

(51) Int. Cl. C12P 19/04; C07K 14/195; C12N 1/14; C12N 1/20; C12N 9/10; C12N 9/12; C12R 1/01; C12R 1/645

(52) U.S. Cl. CPC C12P 19/04; C07K 14/195; C12N 1/14; C12N 1/20; C12N 9/1048; C12N 9/1205; C12R 2001/01; C12R 2001/645

(57) ABSTRACT

The present invention relates to a genetically modified cell comprising a recombinant nucleic acid sequence and/or a cluster of recombinant nucleic acid sequences encoding a transporter protein and/or a cluster of proteins capable of importing an acceptor oligosaccharide of at least three or four monosaccharide units into said cell, wherein said transporter protein and/or cluster of proteins is selected from the group consisting of mutated lactose permease and ABC-importers or MFS importers from a gram-positive bacterium.

The present invention further relates to a method for producing a oligosaccharide, such as a human milk oligosaccharide (HMO), having at least four monosaccharide units, said method comprising culturing a genetically modified cell according to the present invention in a culture medium with a suitable carbon-source and said acceptor oligosaccharide of at least three monosaccharide units; producing said human milk oligosaccharide (HMO) having at least four monosaccharide units by said genetically modified cell, and retrieving the human milk oligosaccharide (HMO) having at least four monosaccharide units from the culture medium and/or the genetically modified cell.

### CROSS-REFERENCE TO RELATED APPLICATIONS

This application is a national stage entry pursuant to 35 U.S.C. § 371 of International Application No. PCT/EP2022/084101, filed on Dec. 1, 2022, which claims priority to Denmark Application No. PA202170594, filed on Dec. 1, 2021, the entire contents of all of which are hereby incorporated by reference in their entirety.

### SEQUENCE LISTING

The computer-readable Sequence Listing submitted on Aug. 15, 2025 and identified as follows: 153,267 bytes ST.26 XML document file named “Sequence Listing.xml,” created Aug. 14, 2025, is incorporated herein by reference in its entirety.

### FIELD

The present invention relates to the production of complex oligosaccharides, such as Human Milk Oligosaccharides (HMOs) from complex precursor oligosaccharides and the genetic engineering of suitable cells for use in said production, as well as to methods for producing said complex HMOs and their precursors.

### BACKGROUND

The design and construction of bacterial cell factories to produce complex Human Milk Oligosaccharides (HMOs) consisting of 3-6 monosaccharide units is of paramount importance to provide innovative and scalable solutions for the more complex products of tomorrow.

To this direction, rational strain engineering principles are commonly applied to single bacterial cells. Such principles usually refer to a) the introduction of a desired biosynthetic pathway to the host, b) the increase of the cellular pools of relevant activated sugars required as donors in the desired reactions, c) the enhancement of lactose import by the native lactose permease LacY and d) the introduction of a heterologous sugar efflux transporter to export the desired newly formed oligosaccharide (for review see Bych et al 2019 Current Opinion in Biotechnology 56:130-137).

Although these approaches usually involve the import of lactose (substrate) by the native lactose permease LacY of E. coli and its further decoration in the cell to form more complex HMOs varying from tri- to hexa-saccharides, there are very few disclosures of the import of oligosaccharides with more than two units by a single cell.

WO2015/032413 describes an internalization mechanism for a trisaccharide acceptor, mentioning LacY as the only specific example of such a transport mechanism. LacY is however naturally present in E. coli cells and neither selective nor highly effective for importing trisaccharides.

EP3848471 describes the use of a saccharide importer for the uptake of an intermediate oligosaccharide consisting of at least three monosaccharide moieties. The application however fails to identify any specific importers capable of transporting an oligosaccharide with three monosaccharides.

### SUMMARY OF THE INVENTION

The present invention relates to a genetically modified cell comprising a recombinant nucleic acid sequence and/or a cluster of recombinant nucleic acid sequences encoding a transporter protein and/or a cluster of proteins capable of importing an acceptor oligosaccharide of at least 3, such as at least 4 or 5, monosaccharide units into said cell, wherein said transporter protein and/or cluster of proteins is selected from the group consisting of mutated LacY, as shown in table 2, and ABC-importers or MFS importers from a gram-positive bacterium, as shown in table 1.

The genetically modified cell according to the present invention can further comprise at least one recombinant nucleic acid sequence encoding at least one glycosyltransferase capable of transferring a glycosyl residue from a glycosyl donor to said acceptor oligosaccharide to synthesize an oligosaccharide product, such as an HMO, having at least four monosaccharide units.

The glycosyltransferase is typically selected from the group consisting of fucosyltransferases, galactosyltransferases, glucosaminyltransferases, sialic acid transferases, N-acetylglucosaminyl transferases and N-acetylglucosaminyl transferases. In one aspect, the glycosyltransferase is selected from the beta-1,4-galactosyltransferases or beta-1,3-galactosyltransferases in table 3.

The genetically modified cell according to the present invention can comprise one or more pathways to produce nucleotide-activated sugar selected from the group consisting of glucose-UDP-GlcNac, GDP-fucose, UDP-galactose, UDP-glucose, UDP-N-acetylglucosamine, UDP-N-acetylgalactosamine and CMP-N-acetylneuraminic acid.

The genetically modified cell according to the present invention can further comprise a nucleic acid sequence encoding an MFS transporter protein capable of exporting the human milk oligosaccharide product having at least four monosaccharide units into the extracellular medium, such as Vag.

The present invention also relates to a method for producing an oligosaccharide, such as a human milk oligosaccharide (HMO), having at least four monosaccharide units, said method comprising culturing a genetically modified cell according to the present invention in the presence of an acceptor oligosaccharide of at least three monosaccharide units.

The present invention thus relates to a method for producing an oligosaccharide, such as a human milk (HMO), having at least four monosaccharide units, said method comprising culturing a genetically modified cell comprising:
a recombinant nucleic acid sequence and/or a cluster of recombinant nucleic acid sequences encoding a transporter protein and/or a cluster of proteins capable of importing an acceptor oligosaccharide of at least three monosaccharide units into said cell, and
at least one recombinant nucleic acid encoding at least one glycosyltransferase capable of transferring a glycosyl residue from a glycosyl donor to said acceptor oligosaccharide to synthesize a human milk oligosaccharide product having at least four monosaccharide units,
wherein the recombinant nucleic acid sequence and/or the cluster of recombinant nucleic acid sequences encoding a transporter protein and/or a cluster of proteins capable of importing said acceptor oligosaccharide of at least three monosaccharide units is selected from the group consisting of mutated lactose permease, as shown in table 2and ABC-importers or MFS importers from a gram-positive bacterium, as shown in table 1.

In one embodiment of the method, the acceptor oligosaccharide having at least three or four or five monosaccharide units is a neutral oligosaccharide, which is added to the culture medium in which the genetically modified cell is cultured. The acceptor oligosaccharide is internalized from the culture medium by the genetically modified cell which produces the oligosaccharide of at least four monosaccharide units.

In one embodiment, the acceptor oligosaccharide does not contain a fucosyl unit.

Typically, the acceptor oligosaccharide having at least three monosaccharide units is LNTII, 2′FL or 3FL and the acceptor oligosaccharide having at least four monosaccharide units is LNT or LNnT.

In one embodiment, the method according to the present invention produces an oligosaccharide of four monosaccharide units, such as human milk oligosaccharide selected from the group consisting of LNT, LNnT, DFL or SFL.

In one embodiment, the method according to the present invention produces an oligosaccharide of five monosaccharide units, such as a human milk oligosaccharide (HMO) selected from the group consisting of LNFP-I, LNFP-II, LNFP-III, LNFP-V, LNFP-VI, LST-a, LST-b and LST-c.

In one embodiment, the method according to the present invention produces a oligosaccharide of six monosaccharide units, such as a human milk oligosaccharide (HMO) selected from the list consisting of LNH, LNnH, pLNnH, pLNH-1, DSLNT and LNDFH-1, LNDFH-II and LNDFH-III.

Various exemplary embodiments and details are described hereinafter, with reference to the figures and sequences when relevant. It should also be noted that the figures are only intended to facilitate the description of the embodiments. They are not intended as an exhaustive description of the disclosure or as a limitation on the scope of the disclosure. In addition, an illustrated embodiment needs not have all the aspects or advantages shown. An aspect or an advantage described in conjunction with a particular embodiment is not necessarily limited to that embodiment and can be practiced in any other embodiments even if not so illustrated, or if not so explicitly described.

### DESCRIPTION OF THE FIGURES

FIG. 1. % Difference in LNnT formation (i.e., adjusted LNnT values) for strains expressing a transporter protein of the present invention (MFS, ABC-transporter, or LacY mutant variant) relative to the control strain MP1 that does not express a recombinant importer protein.

### DETAILED DESCRIPTION

The present invention approaches the biotechnological challenge of in vivo oligosaccharide production, in particular human milk oligosaccharide (HMO) production, namely the import of oligosaccharides that contain at least three monosaccharide units and its further decoration in the microbial host cell, such as an E. coli, Bacillus sp, or yeast cell, to generate more complex oligosaccharides, such as HMOs, of four to six monosaccharide units. The present invention offers specific strain engineering solutions to produce complex oligosaccharides, such as HMOs, by exploiting the potential of importer proteins identified in Gram-positive (Gram+) bacteria, as well as mutant variants of a lactose permease protein.

In other words, microbial stains, such as E. coli, Bacillus sp, or yeast strains or other species described in the section “host cells”, covered by the present invention express genes encoding key enzymes for oligosaccharide, such as HMO, biosynthesis along with one or more genes encoding a mutant variant of the E. coli LacY protein (Table 2) and/or an ABC and/or MFS transporter originating from a Gram+ bacterium (Table 1) to import a precursor oligosaccharide molecule (acceptor oligosaccharide), which has three or more monosaccharide units, such as four monosaccharide units, such as five monosaccharide units, and is further decorated by recombinant enzyme(s) within the cell to produce even more complex molecules, such as oligosaccahrides, in particular HMOs, in the cell.

The advantage of importing an acceptor oligosaccharide of at least three or four or five monosaccharide units into the host cell instead of enabling the host cell to make the acceptor oligosaccharide from lactose as it is conventionally done (see for example WO 2020/115671), is that only one and maximum two glycosyltransferases need(s) to be expressed by the genetically modified cell to make the complex oligosaccharide, e.g., HMO. Typically, by-products are either the major (product) HMO precursors (such as lactose or other acceptor oligosaccharides) or products of further modification of the major (product) HMO. The fewer glycosyltransferases that are needed to generate the major HMO (product HMO), the fewer by-product HMOs or other impurities will be generated, and the purity of the complex HMO (major HMO) will therefore increase. For example, if LNFP-I is produced from lactose in a single cell, three glycosyl transferases are required namely, β-1,3-N-acetylglucosaminyl-transferase forming LNT-II, a β-1,3-galactosyltransferase forming LNT and an alpha-1,2-fucosyl-transferase forming LNFP-I. In this case the desired(product) HMO is LNFP-I, and LNT-II, LNT, 2′-FL and DFL are likely HMO by-products. With the genetically modified cell of the present invention, which is capable of importing LNT, only the alpha-1,2-fucosyl-transferase will be needed in the genetically modified HMO producing cell and no lactose will be present, thereby avoiding undesired HMOs (LNT-II, 2′-FL and DFL) and other impurities (e.g., Gal-LNT, Gal-Lac, GlcNAc-LNT) as by-products and allowing partial or full conversion of LNT to LNFP-I towards the end of fermentation, since LNT is the substrate fed to the genetically modified cell.

There are several applications of biotechnological interest, where the concept of the present invention could be relevant. In the following sections, such applications are described in more detail.

ABC and MFS transporters of gram-positive origin The genetically modified cell according to the present invention comprises at least one recombinant nucleic acid sequence encoding and/or a cluster of recombinant nucleic acid sequences encoding a recombinant transporter protein and/or a cluster of recombinant proteins capable of importing an acceptor oligosaccharide of at least 3, such as at least 4, such as at least 5, monosaccharide units into said cell.

The present invention offers specific strain engineering solutions to produce complex oligosaccharides, such as complex HMOs, by exploiting the potential of importer proteins identified in Gram-positive (Gram+) bacteria, and in particular members of the Bifidobacterium, Roseburia and Eubacterium species

Table 1 shows MFS-transporter proteins of gram-positive origin and ABC-transporter protein clusters of gram-positive origin capable of importing an acceptor oligosaccharide of at least three or four or five monosaccharide units into a cell. The term transporter and importer may be used interchangeably.

The acceptor oligosaccharide is preferably a precursor for a more complex oligosaccharide, such as more complex HMO. In table 1 it is indicated which acceptor oligosaccharide the transporter is expected to import into the cell. Acceptor oligosaccharides are further described in the section “An acceptor oligosaccharide of at least three or four or five monosaccharide units”

TABLE 1
ABC- and MFS-transporters from gram-positive bacteria with an indication
of the precursor oligosaccharide the transporter is expected to import.
The ABC transporters are composed of three to four genes. For ease of
reference each transporter has been given a transporter ID (TP ID)

| | | | | SEQ | Expected | |
|---|---|---|---|---|---|---|
| TP | TP | | | ID | oligosaccharide | |
| ID | type | Gene Name | GenBank ID | NO: | transport | Origin |
| 1 | MFS | BIon_0247 | ACJ51375.1 | 2 | LNnT | B. infantis ATCC |
| | | | | | | 15697 = JCM |
| | | | | | | 1222 |
| 2 | MFS | BIon_0431 | ACJ51548.1 | 6 | oligosaccharide | B. infantis ATCC |
| | | | | | | 15697 = JCM |
| | | | | | | 1222 |
| 3 | MFS | BIon_0788 | ACJ51891.1 | 7 | oligosaccharide | B. infantis ATCC |
| | | | | | | 15697 = JCM |
| | | | | | | 1222 |
| 13 | MFS | BIon_0962 | ACJ52061.1 | 12 | Fucosylated- | B. infantis ATCC |
| | | | | | HMO | 15697 = JCM |
| | | | | | | 1222 |
| 14 | MFS | BIon_2307 | ACJ53365.1 | 19 | Fucosylated- | B. infantis ATCC |
| | | | | | HMO | 15697 = JCM |
| | | | | | | 1222 |
| 4 | MFS | BIon_2400 | ACJ53455.1 | 27 | oligosaccharide | B. infantis ATCC |
| | | | | | | 15697 = JCM |
| | | | | | | 1222 |
| 5 | ABC | BIon_2341 | ACJ53399.1 | 20 | LN(n)T | B. infantis ATCC |
| | | | | | | 15697 = JCM |
| | | | | | | 1222 |
| | | BIon_2342 | ACJ53400.1 | 21 | | B. infantis ATCC |
| | | | | | | 15697 = JCM |
| | | | | | | 1222 |
| | | BIon_2343 | ACJ53401.1 | 22 | | B. infantis ATCC |
| | | | | | | 15697 = JCM |
| | | | | | | 1222 |
| | | BIon_2344 | ACJ53402.1 | 23 | | B. infantis ATCC |
| | | | | | | 15697 = JCM |
| | | | | | | 1222 |
| 6 | ABC | BIon_2345 | ACJ53403.1 | 24 | LN(n)T | B. infantis ATCC |
| | | | | | | 15697 = JCM |
| | | | | | | 1222 |
| | | BIon_2346 | ACJ53404.1 | 25 | | B. infantis ATCC |
| | | | | | | 15697 = JCM |
| | | | | | | 1222 |
| | | BIon_2347 | ACJ53405.1 | 26 | | B. infantis ATCC |
| | | | | | | 15697 = JCM |
| | | | | | | 1222 |
| 15 | ABC | BIon_0341/ | ACJ51465.1/ | 3 | 2′-FL, 3-FL, | B. infantis ATCC |
| | | BIon_2204 | ACJ53264.1 | 18 | DFL, LNFP-I | 15697 = JCM |
| | | | | | | 1222 |
| | | BIon_0342/ | ACJ51466.1/ | 4 | | B. infantis ATCC |
| | | BIon_2203 | ACJ53263.1 | 17 | | 15697 = JCM |
| | | | | | | 1222 |
| | | BIon_2202 | ACJ53262.1 | 16 | | B. infantis ATCC |
| | | | | | | 15697 = JCM |
| | | | | | | 1222 |
| 16 | ABC | BIon_0341/ | ACJ51465.1/ | 3 | 2′-FL, 3-FL, | B. infantis ATCC |
| | | BIon_2204 | ACJ53264.1 | 18 | DFL, LNFP-I | 15697 = JCM |
| | | | | | | 1222 |
| | | BIon_0342/ | ACJ51466.1/ | 4 | | B. infantis ATCC |
| | | BIon_2203 | ACJ53263.1 | 17 | | 15697 = JCM |
| | | | | | | 1222 |
| | | BIon_0343 | ACJ51467.1 | 5 | | B. infantis ATCC |
| | | | | | | 15697 = JCM |
| | | | | | | 1222 |
| 7 | ABC | BIon_0883 | ACJ51983.1 | 8 | LN(n)T, Lewis a, | B. infantis ATCC |
| | | | | | type 1 H- | 15697 = JCM |
| | | | | | trisaccharide | 1222 |
| | | BIon_0884 | ACJ51984.1 | 9 | | B. infantis ATCC |
| | | | | | | 15697 = JCM |
| | | | | | | 1222 |
| | | BIon_0885 | ACJ51985.1 | 10 | | B. infantis ATCC |
| | | | | | | 15697 = JCM |
| | | | | | | 1222 |
| | | BIon_0886 | ACJ51986.1 | 11 | | B. infantis ATCC |
| | | | | | | 15697 = JCM |
| | | | | | | 1222 |
| 8 | ABC | BIon_2177 | ACJ53238.1 | 15 | LNB, LN(n)T, | B. infantis ATCC |
| | | | | | LNO, LNH, | 15697 = JCM |
| | | | | | sialyl | 1222 |
| | | BIon_2176 | ACJ53237.1 | 14 | | B. infantis ATCC |
| | | | | | | 15697 = JCM |
| | | | | | | 1222 |
| | | BIon_2175 | ACJ53236.1 | 13 | LNT | B. infantis ATCC |
| | | | | | | 15697 = JCM |
| | | | | | | 1222 |
| 9 | ABC | RHOM_04095 | AEN95941.1 | 35 | LNT | R. hominis A2- |
| | | | | | | 183 |
| | | RHOM_04100 | AEN95942.1 | 36 | | R. hominis A2- |
| | | | | | | 183 |
| | | RHOM_04105 | AEN95943.1 | 37 | | R. hominis A2- |
| | | | | | | 183 |
| 10 | ABC | HMPREF0373_02960 | ERK42291.1 | 38 | LNT | E. ramulus ATCC |
| | | | | | | 29099 |
| | | HMPREF0373_02961 | ERK42292.1 | 39 | | E. ramulus ATCC |
| | | | | | | 29099 |
| | | HMPREF0373_02962 | ERK42293.1 | 40 | | E. ramulus ATCC |
| | | | | | | 29099 |
| 17 | ABC | BBKW_1838/ | BAQ29973.1/ | 44 | 2-′FL, 3-FL, | B. |
| | | BKAS_1807 | KFI63925.1 | 43 | DFL, LNFP-I | kashiwanohense |
| | | | | | (FL-2) | JCM 15439 |
| | | BBKW_1839/ | BAQ29974.1/ | 45 | | B. |
| | | BKAS_1806 | KFI63924.1 | 42 | | kashiwanohense |
| | | | | | | JCM 15439 |
| | | BBKW_1840/ | BAQ29975.1/ | 46 | | B. |
| | | BKAS_1805 | KFI63923.1 | 41 | | kashiwanohense |
| | | | | | | JCM 15439 |
| 18 | ABC | BBPC_1775 | BAR04453.1 | 47 | 2′-FL, 3-FL, | B. |
| | | | | | LNT-II, DFL, | pseudocatenulatum |
| | | | | | LNFP-I | JCM 1200 |
| | | BBPC_1776 | BAR04454.1 | 48 | LNT-II, LN(n)T | B. |
| | | | | | | pseudocatenulatum |
| | | | | | | JCM 1200 |
| | | BBPC_1777 | BAR04455.1 | 49 | | B. |
| | | | | | | pseudocatenulatum |
| | | | | | | JCM 1200 |
| 11 | ABC | BBR_0527/IntP1 | ABE95224.1 | 28 | | B. breve |
| | | | | | | UCC2003 |
| | | BBR_0528/IntP2 | ABE95225.1 | 29 | | B. breve |
| | | | | | | UCC2003 |
| | | BBR_0530/IntS | ABE95226.1 | 30 | | B. breve |
| | | | | | | UCC2003 |
| | | BBR_0531 | ABE95228.1 | 50 | | B. breve |
| | | | | | | UCC2003 |
| 12 | ABC | BBR_1554/nahS | ABE96225.1 | 31 | LNnT | B. breve |
| | | | | | | UCC2003 |
| | | BBR_1558 | ABE96228.1 | 32 | | B. breve |
| | | | | | | UCC2003 |
| | | BBR_1559 | ABE96229.1 | 33 | | B. breve |
| | | | | | | UCC2003 |
| | | BBR_1560 | ABE96230.1 | 34 | | B. breve |
| | | | | | | UCC2003 |

TABLE 14
Nucleic acid sequences encoding the ABC- and MFS-transporters from
gram-positive bacteria in Table 1. For ease of reference each
transporter has been given a transporter ID (TP ID or just TP)

| | | | SEQ | Expected | |
|---|---|---|---|---|---|
| TP | TP | | ID | oligosaccharide | |
| ID | type | Gene Name | NO: | transport | Origin |
| 1 | MFS | BIon_0247 | 64 | LNnT | Artificial -Codon |
| | | | | | optimized |
| 2 | MFS | BIon_0431 | 65 | oligosaccharide | Artificial -Codon |
| | | | | | optimized |
| 3 | MFS | BIon_0788 | 66 | oligosaccharide | Artificial -Codon |
| | | | | | optimized |
| 13 | MFS | BIon_0962 | 67 | Fucosylated- | Artificial -Codon |
| | | | | HMO | optimized |
| 14 | MFS | BIon_2307 | 68 | Fucosylated- | Artificial -Codon |
| | | | | HMO | optimized |
| 4 | MFS | BIon_2400 | 63 | oligosaccharide | Artificial -Codon |
| | | | | | optimized |
| 5 | ABC | BIon_2341 | 59 | LN(n)T | B. infantis ATCC |
| | | BIon_2342 | | | 15697 = JCM 1222 |
| | | BIon_2343 | | | |
| | | BIon_2344 | | | |
| 6 | ABC | BIon_2345 | 60 | LN(n)T | B. infantis ATCC |
| | | BIon_2346 | | | 15697 = JCM 1222 |
| | | BIon_2347 | | | |
| 15 | ABC | BIon_0341/BIon_2204 | 69 | 2′-FL, 3-FL, | B. infantis ATCC |
| | | BIon_0342/BIon_2203 | | DFL, LNFP-I | 15697 = JCM 1222 |
| | | BIon_2202 | | | |
| 16 | ABC | BIon_0341/BIon_2204 | 52 | 2′-FL, 3-FL, | B. infantis ATCC |
| | | BIon_0342/BIon_2203 | | DFL, LNFP-I | 15697 = JCM 1222 |
| | | BIon_0343 | | | |
| 7 | ABC | BIon_0883 | 58 | LN(n)T, Lewis a, | B. infantis ATCC |
| | | BIon_0884 | | type 1 H- | 15697 = JCM 1222 |
| | | BIon_0885 | | trisaccharide | |
| | | BIon_0886 | | | |
| 8 | ABC | BIon_2177 | 56 | LNB, LN(n)T, | Artificial -Codon |
| | | BIon_2176 | | LNO, LNH, sialyl | optimized |
| | | BIon_2175 | | LNT | |
| 9 | ABC | RHOM_04095 | 53 | LNT | Artificial -Codon |
| | | RHOM_04100 | | | optimized |
| | | RHOM_04105 | | | |
| 10 | ABC | HMPREF0373_02960 | 61 | LNT | Artificial -Codon |
| | | HMPREF0373_02961 | | | optimized |
| | | HMPREF0373_02962 | | | |
| 17 | ABC | BBKW_1838/ | 62 | 2′-FL, 3-FL, | Artificial -Codon |
| | | BKAS_1807 | | DFL, LNFP-I | optimized |
| | | BBKW_1839/ | | (FL-2) | |
| | | BKAS_1806 | | | |
| | | BBKW_1840/ | | | |
| | | BKAS_1805 | | | |
| 18 | ABC | BBPC_1775 | 54 | 2′-FL, 3-FL, | Artificial -Codon |
| | | BBPC_1776 | | DFL, LNFP-I | optimized |
| | | BBPC_1777 | | | |
| 11 | ABC | BBR_0527/IntP1 | 55 | LN(n)T | Artificial -Codon |
| | | BBR_0528/IntP2 | | | optimized |
| | | BBR_0530/IntS | | | |
| | | BBR_0531 | | | |
| 12 | ABC | BBR_1554/nahS | 57 | LNnT | B. breve UCC2003 |
| | | BBR_1558 | | | |
| | | BBR_1559 | | | |
| | | BBR_1560 | | | |

The present invention relates to a genetically modified cell comprising a cluster of recombinant nucleic acid sequences encoding a cluster of proteins capable of importing an acceptor oligosaccharide of at least three or at least four or at least five monosaccharide units into said cell, wherein said cluster of proteins is an ABC transporter from a gram-positive cell. In particular, an ABC transporter as listed in table1, in particular a transporter selected from the group consisting of TP ID: 5, 6, 7, 8, 9, 10, 11, 12, 15, 16, 17 and 18 or a subset of ABC transporters selected from the group consisting of TP ID: 8, 9, 10,11, 17 and 18.

In an embodiment, the cluster of transporter proteins capable of importing an acceptor oligosaccharide of at least three monosaccharide units into said cell is the cluster comprising Blon_2341, Blon_2342, Blon_2343, Blon_2344 (TP5 in table 1). In particular an ABC transporter formed of four sub-units with the amino acid sequences comprising or consisting of SEQ ID NO: 20, 21, 22 and 23 or amino acid sequences with at least 80%, such as 85%, such as 90%, such as 95% identity to SEQ ID: 20, 21, 22 and 23. In a further embodiment the cluster of recombinant nucleic acid sequences encoding the cluster comprising Blon_2341, Blon_2342, Blon_2343 and Blon_2344 (TP5) comprises or consists of SEQ ID NO: 59 or a nucleic acid sequence with at least 80%, such as 85%, such as 90%, such as 95% identity to SEQ ID NO: 59.

In an embodiment, the cluster of transporter proteins capable of importing an acceptor oligosaccharide of at least three monosaccharide units into said cell is the cluster comprising Blon_2345, Blon_2346 and Blon_2347 (TP6 in table 1). In particular an ABC transporter formed of three sub-units with the amino acid sequences comprising or consisting of SEQ ID NO: 24, 25 and 26 or amino acid sequences with at least 80%, such as 85%, such as 90%, such as 95% identity to SEQ ID: 24, 25 and 26. In a further embodiment the cluster of recombinant nucleic acid sequences encoding the cluster comprising Blon_2345, Blon_2346 and Blon_2347 (TP6) comprises or consists of SEQ ID NO: 60 or a nucleic acid sequence with at least 80%, such as 85%, such as 90%, such as 95% identity to SEQ ID NO: 60.

In an embodiment, the cluster of transporter proteins capable of importing an acceptor oligosaccharide of at least three monosaccharide units into said cell is the cluster comprising Blon_0341/Blon_2204, Blon_0342/Blon_2203 and Blon_2202 (TP15 in table 1). In particular an ABC transporter formed of three sub-units with the amino acid sequences comprising or consisting of SEQ ID NO: 3 or 18, 4 or 17 and 16 or amino acid sequences with at least 80%, such as 85%, such as 90%, such as 95% identity to SEQ ID: 3 or 18, 4 or 17 and 16. In a further embodiment the cluster of recombinant nucleic acid sequences encoding the cluster comprising Blon_0341/Blon_2204, Blon_0342/Blon_2203 and Blon_2202 (TP15) comprises or consists of SEQ ID NO: 69 or a nucleic acid sequence with at least 80%, such as 85%, such as 90%, such as 95% identity to SEQ ID NO: 69.

In an embodiment, the cluster of transporter proteins capable of importing an acceptor oligosaccharide of at least three monosaccharide units into said cell is the cluster comprising Blon_0341/Blon_2204, Blon_0342/Blon_2203 and Blon_0343 (TP16 in table 1). In particular an ABC transporter formed of three sub-units with the amino acid sequences comprising or consisting of SEQ ID NO: 3 or 18, 4 or 17 and 5 or amino acid sequences with at least 80%, such as 85%, such as 90%, such as 95% identity to SEQ ID: 3 or 18, 4 or 17 and 5. In a further embodiment the cluster of recombinant nucleic acid sequences encoding the cluster comprising Blon_0341/Blon_2204, Blon_0342/Blon_2203 and Blon_0343 (TP16) comprises or consists of SEQ ID NO: 52 or a nucleic acid sequence with at least 80%, such as 85%, such as 90%, such as 95% identity to SEQ ID NO: 52.

In an embodiment, the cluster of transporter proteins capable of importing an acceptor oligosaccharide of at least four monosaccharide units into said cell is the cluster comprising Blon_0883, Blon_0884, Blon_0885 and Blon_0886 (TP7 in table 1). In particular an ABC transporter formed of three sub-units with the amino acid sequences comprising or consisting of SEQ ID NO: 8, 9, 10 and 11 or amino acid sequences with at least 80%, such as 85%, such as 90%, such as 95% identity to SEQ ID: 8, 9, 10 and 11. In a further embodiment the cluster of recombinant nucleic acid sequences encoding the cluster comprising Blon_0883, Blon_0884, Blon_0885 and Blon_0886 (TP7) comprises or consists of SEQ ID NO: 58 or a nucleic acid sequence with at least 80%, such as 85%, such as 90%, such as 95% identity to SEQ ID NO: 58.

In an embodiment, the cluster of transporter proteins capable of importing an acceptor oligosaccharide of at least four monosaccharide units into said cell is the cluster comprising BBR_1554/nahS, BBR_1558, BBR_1559 and BBR_1560 (TP12 in table 1). In particular an ABC transporter formed of three sub-units with the amino acid sequences comprising or consisting of SEQ ID NO: 31, 32, 33 and 34 or amino acid sequences with at least 80%, such as 85%, such as 90%, such as 95% identity to SEQ ID: 31, 32, 33 and 34. In a further embodiment the cluster of recombinant nucleic acid sequences encoding the cluster comprising BBR_1554/nahS, BBR 1558. BBR_1559 and BBR_1560 (TP12) comprises or consists of SEQ ID NO: 57 or a nucleic acid sequence with at least 80%, such as 85%, such as 90%, such as 95% identity to SEQ ID NO: 57

In an embodiment, the cluster of transporter proteins capable of importing an acceptor oligosaccharide of at least three monosaccharide units into said cell is the cluster comprising Blon_2177, 2176 and 2175 (TP8 in table 1). In particular an ABC transporter formed of three sub-units with the amino acid sequences comprising or consisting of SEQ ID NO: 13, 14 and 15 or amino acid sequences with at least 80%, such as 85%, such as 90%, such as 95% identity to SEQ ID: 13, 14 and 15. In a further embodiment the cluster of recombinant nucleic acid sequences encoding the cluster comprising Blon_2177, 2176 and 2175 (TP8) comprises or consists of SEQ ID NO: 56 or a nucleic acid sequence with at least 80%, such as 85%, such as 90%, such as 95% identity to SEQ ID NO: 56.

In another embodiment, the cluster of transporter proteins capable of importing an acceptor oligosaccharide of at least three monosaccharide units into said cell is the cluster comprising RHOM_04095, 04100, 04105 (TP9 in table 1). In particular an ABC transporter formed of three sub-units with the amino acid sequences comprising or consisting of SEQ ID NO: 35, 36 and 37 or amino acid sequences with at least 80%, such as 85%, such as 90%, such as 95% identity to SEQ ID: 35, 36 and 37. In a further embodiment the cluster of recombinant nucleic acid sequences encoding the cluster comprising RHOM_04095, 04100, 04105 (TP9) comprises or consists of SEQ ID NO: 53 or a nucleic acid sequence with at least 80%, such as 85%, such as 90%, such as 95% identity to SEQ ID NO: 53.

In another embodiment, the cluster of transporter proteins capable of importing an acceptor oligosaccharide of at least three monosaccharide units into said cell is the cluster comprising BBPC_1775, 1776, 1777 (TP18 in table 1). In particular an ABC transporter formed of three sub-units with the amino acid sequences comprising or consisting of SEQ ID NO: 47, 48 and 49 or amino acid sequences with at least 80%, such as 85%, such as 90%, such as 95% identity to SEQ ID: 47, 48 and 49. In a further embodiment the cluster of recombinant nucleic acid sequences encoding the cluster comprising BBPC_1775, 1776, 1777 (TP18) comprises or consists of SEQ ID NO: 54 or a nucleic acid sequence with at least 80%, such as 85%, such as 90%, such as 95% identity to SEQ ID NO: 54.

In another embodiment, the cluster of transporter proteins capable of importing an acceptor oligosaccharide of at least four monosaccharide units into said cell is the cluster comprising BBR_0527, 0528, 0530, 0531 (TP11 in table 1). In particular an ABC transporter formed of four sub-units with the amino acid sequences comprising or consisting of SEQ ID NO: 28, 29, 30 and 50 or amino acid sequences with at least 80%, such as 85%, such as 90%, such as 95% identity to SEQ ID: 28, 29, 30 and 50. In a further embodiment the cluster of recombinant nucleic acid sequences encoding the cluster comprising BBR 0527, 0528, 0530, 0531 (TP11) comprises or consists of SEQ ID NO: 55 or a nucleic acid sequence with at least 80%, such as 85%, such as 90%, such as 95% identity to SEQ ID NO: 55.

In another embodiment, the cluster of transporter proteins capable of importing an acceptor oligosaccharide of at least three monosaccharide units into said cell is the cluster comprising HMPREF0373_02960, 0373_02961, 0373_02962 (TP10 in table 1). In particular an ABC transporter formed of three sub-units with the amino acid sequences comprising or consisting of SEQ ID NO: 38, 39 and 40 or amino acid sequences with at least 80%, such as 85%, such as 90%, such as 95% identity to SEQ ID: 38, 39 and 40. In a further embodiment the cluster of recombinant nucleic acid sequences encoding the cluster comprising HMPREF0373_02960, 0373_02961, 0373_02962 (TP10) comprises or consists of SEQ ID NO: 61 or a nucleic acid sequence with at least 80%, such as 85%, such as 90%, such as 95% identity to SEQ ID NO: 61.

In another embodiment, the cluster of transporter proteins capable of importing an acceptor oligosaccharide of at least three monosaccharide units into said cell is the cluster comprising BBKW_1838, 1839, 1840 (TP17 in table 1). In particular an ABC transporter formed of three sub-units with the amino acid sequences comprising or consisting of SEQ ID NO: 44, 45 and 46 or amino acid sequences with at least 80%, such as 85%, such as 90%, such as 95% identity to SEQ ID: 44, 45 and 46 or with the amino acid sequences comprising or consisting of SEQ ID NO: 41, 42 and 43 or amino acid sequences with at least 80%, such as 85%, such as 90%, such as 95% identity to SEQ ID: 41, 42 and 43. In a further embodiment the cluster of recombinant nucleic acid sequences encoding the cluster comprising BBKW_1838, 1839, 1840 (TP17) comprises or consists of SEQ ID NO: 62 or a nucleic acid sequence with at least 80%, such as 85%, such as 90%, such as 95% identity to SEQ ID NO: 62.

The present invention relates to a genetically modified cell comprising a recombinant nucleic acid sequence encoding a transporter protein capable of importing an acceptor oligosaccharide of at least three or at least four monosaccharide units into said cell, wherein said transporter protein is an MFS transporter from a gram-positive cell. In particular an MFS transporter as listed in table1, in particular a transporter selected from the group consisting of TP ID: 1, 2, 3, 4, 13 and 14.

In one embodiment, the MFS transporter proteins capable of importing an acceptor oligosaccharide of at least three monosaccharide units into said cell is Blon:0247 (TP1 in table 1). In particular a MFS transporter protein with an amino acid sequence comprising or consisting of SEQ ID NO: 2 or an amino acid sequence with at least 80%, such as 85%, such as 90%, such as 95% identity to SEQ ID: 2. In a further embodiment the recombinant nucleic acid sequences encoding Blon:0247 (TP1) comprises or consists of SEQ ID NO: 64 or a nucleic acid sequence with at least 80%, such as 85%, such as 90%, such as 95% identity to SEQ ID NO: 64.

In another embodiment, the MFS transporter proteins capable of importing an acceptor oligosaccharide of at least three monosaccharide units into said cell is Blon_0431 (TP2 in table 1). In particular a MFS transporter protein with an amino acid sequence comprising or consisting of SEQ ID NO: 6 or an amino acid sequence with at least 80%, such as 85%, such as 90%, such as 95% identity to SEQ ID: 6. In a further embodiment the recombinant nucleic acid sequences encoding Blon_0431 (TP2) comprises or consists of SEQ ID NO: 65 or a nucleic acid sequence with at least 80%, such as 85%, such as 90%, such as 95% identity to SEQ ID NO: 65.

In another embodiment, the MFS transporter proteins capable of importing an acceptor oligosaccharide of at least three monosaccharide units into said cell is Blon_0788 (TP3 in table 1). In particular a MFS transporter protein with an amino acid sequence comprising or consisting of SEQ ID NO: 7 or an amino acid sequence with at least 80%, such as 85%, such as 90%, such as 95% identity to SEQ ID: 7. In a further embodiment the recombinant nucleic acid sequences encoding Blon_0788 (TP3) comprises or consists of SEQ ID NO: 66 or a nucleic acid sequence with at least 80%, such as 85%, such as 90%, such as 95% identity to SEQ ID NO: 66.

In another embodiment, the MFS transporter proteins capable of importing an acceptor oligosaccharide of at least three monosaccharide units into said cell is Blon_0962 (TP13 in table 1). In particular a MFS transporter protein with an amino acid sequence comprising or consisting of SEQ ID NO: 12 or an amino acid sequence with at least 80%, such as 85%, such as 90%, such as 95% identity to SEQ ID: 12. In a further embodiment the recombinant nucleic acid sequences encoding Blon_0962 (TP13) comprises or consists of SEQ ID NO: 67 or a nucleic acid sequence with at least 80%, such as 85%, such as 90%, such as 95% identity to SEQ ID NO: 67.

In another embodiment, the MFS transporter proteins capable of importing an acceptor oligosaccharide of at least three monosaccharide units into said cell is Blon_2307 (TP14 in table 1). In particular a MFS transporter protein with an amino acid sequence comprising or consisting of SEQ ID NO: 19 or an amino acid sequence with at least 80%, such as 85%, such as 90%, such as 95% identity to SEQ ID: 19. In a further embodiment the recombinant nucleic acid sequences encoding Blon_2307 (TP14) comprises or consists of SEQ ID NO: 68. or a nucleic acid sequence with at least 80%, such as 85%, such as 90%, such as 95% identity to SEQ ID NO: 68.

In another embodiment, the MFS transporter proteins capable of importing an acceptor oligosaccharide of at least three monosaccharide units into said cell is Blon 2400 (TP4 in table 1). In particular a MFS transporter protein with an amino acid sequence comprising or consisting of SEQ ID NO: 27 or an amino acid sequence with at least 80%, such as 85%, such as 90%, such as 95% identity to SEQ ID: 27. In a further embodiment the recombinant nucleic acid sequences encoding Blon_2400 (TP4) comprises or consists of SEQ ID NO: 63 or a nucleic acid sequence with at least 80%, such as 85%, such as 90%, such as 95% identity to SEQ ID NO: 63.

In the present application the ABC and MFS transporters may also be termed importers.

Mutated lacY

The present invention relates to a genetically modified cell comprising a recombinant nucleic acid sequence encoding a recombinant transporter protein capable of importing an acceptor oligosaccharide of at least three monosaccharide units into said cell, wherein said recombinant transporter protein is a porter protein, such as a mutated lactose permease (LacY) as shown in table 2.

In embodiments the mutated lactose permease is selected from a lactose permease of SEQ ID NO: 1 or a lactose permease with 90% identity to SEQ ID NO: 1, wherein the lactose permease has one or more mutations selected from the group consisting of Y236N, Y236H, S306T, A177V, H322N, 1303F, Y236H+S306T, 177V+Y236H, A177V+1303F, A177V+H322N, A177V+S306T or A177V+Y236N+S306T and wherein the mutation is at the corresponding position in SEQ ID NO: 1.

In preferred embodiments the mutated lactose permease is a lactose permease of SEQ ID NO: 1 with the mutation Y236H.

In another preferred embodiments the mutated lactose permease is a lactose permease of SEQ ID NO: 1 with mutation the following two mutations A177V+S306T.

Lactose permease (LacY) is known in its wild-type form to transport the disaccharide lactose from the cell exterior into the E. coli cell. Mutated variants of LacY have been described to be capable of transporting the trisaccharide maltotriose (Olsen et al 1993 J Bacteriol. 175(19):6269-75). In the present invention these mutants were identified as potential importers of trisaccharides (acceptor oligosaccharides/HMO precursor molecules) of relevance in the HMO production, e.g., 2-fucosyllactose (2′FL), 3-fucosyllactose (3FL), lacto-N-triose (LNT-II).

Typically, the genetically modified cell lacks enzymatic activity liable to degrade the acceptor oligosaccharide of at least three or four monosaccharide units.

### Human Milk Oligosaccharide (HMO)

### Oligosaccharides

In the present context, the term “oligosaccharide” means a sugar polymer containing at least three monosaccharide units, i.e., a tri-, tetra-, penta-, hexa- or higher oligosaccharide. The oligosaccharide can have a linear or branched structure containing monosaccharide units that are linked to each other by interglycosidic linkages. Particularly, the oligosaccharide comprises a lactose residue at the reducing end and one or more naturally occurring monosaccharides of 5-9 carbon atoms selected from aldoses (e.g., glucose, galactose, ribose, arabinose, xylose, etc.), ketoses (e.g., fructose, sorbose, tagatose, etc.), deoxysugars (e.g. rhamnose, fucose, etc.), deoxy-aminosugars (e.g. N-acetyl-glucosamine, N-acetyl-mannosamine, N-acetyl-galactosamine, etc.), uronic acids and ketoaldonic acids (e.g. sialic acid). Preferably, the oligosaccharide is an HMO.

### HMO's

Preferred oligosaccharides of the disclosure are human milk oligosaccharides (HMOs).

The term “human milk oligosaccharide” or “HMO” in the present context means a complex carbohydrate found in human breast milk. The HMOs have a core structure comprising a lactose unit at the reducing end that can be elongated by one or more beta-N-acetyl-lactosaminyl and/or one or more beta-lacto-N-biosyl units, and this core structure can be substituted by an alpha-L-fucopyranosyl and/or an alpha-N-acetyl-neuraminyl (sialyl) moiety. HMO structures are for example disclosed in by Xi Chen in Chapter 4 of Advances in Carbohydrate Chemistry and Biochemistry 2015 vol 72.

HMOs are either neutral or acidic. In this regard, the non-acidic (or neutral) HMOs are devoid of a sialyl residue, and the acidic HMOs have at least one sialyl residue in their structure. The non-acidic (or neutral) HMOs can be fucosylated or non-fucosylated.

Examples of such neutral non-fucosylated HMOs include lacto-N-triose II (LNT-II) lacto-N-tetraose (LNT), lacto-N-neotetraose (LNnT), lacto-N-neohexaose (LNnH), para-lacto-N-neohexaose (pLNnH), para-lacto-N-hexaose (pLNH) and lacto-N-hexaose (LNH).

Examples of neutral fucosylated HMOs include 2′-fucosyllactose (2′FL), lacto-N-fucopentaose I (LNFP-I), lacto-N-difucohexaose I (LNDFH-I), 3-fucosyllactose (3′FL), difucosyllactose (DFL or LDFT), lacto-N-fucopentaose II (LNFP-II), lacto-N-fucopentaose III (LNFP-III), lacto-N-difucohexaose III (LNDFH-III), fucosyl-lacto-N-hexaose II (FLNH-II), lacto-N-fucopentaose V (LNFP-V), lacto-N-difucohexaose II (LNDFH-II), fucosyl-lacto-N-hexaose I (FLNH-I), fucosyl-para-lacto-N-hexaose I (FpLNH-1), fucosyl-para-lacto-N-neohexaose II (F-pLNnH II) and fucosyl-lacto-N-neohexaose (FLNnH).

Examples of acidic HMOs include 3′-sialyllactose (3′SL), 6′-sialyllactose (6′SL), 3-fucosyl-3′-sialyllactose (FSL), 3′-O-sialyllacto-N-tetraose a (LST a), fucosyl-LST a (FLST a), 6′-O-sialyllacto-N-tetraose b (LST b), fucosyl-LST b (FLST b), 6′-O-sialyllacto-N-neotetraose (LST c), fucosyl-LST c (FLST c), 3′-O-sialyllacto-N-neotetraose (LST d), fucosyl-LST d (FLST d), sialyl-lacto-N-hexaose (SLNH), sialyl-lacto-N-neohexaose I (SLNH-1), sialyl-lacto-N-neohexaose II (SLNH-II) and disialyl-lacto-N-tetraose (DSLNT).

In the context of the present invention complex HMOs are composed of at least four monosaccharide units. Preferably, in one embodiment, a complex HMO is one that require at least two different glycosyltransferase activities to be produced from lactose as the starting substrate, e.g. the formation of DFL requires an alpha-1,2-fucosyltransferase and an alpha-1,3-fucosyltransferase activity.

In one method according to the present invention, the oligosaccharide produced by the genetically modified cell has at least four monosaccharide units. Preferably, the oligosaccharide is a human milk oligosaccharide (HMO) of only four monosaccharide units selected from LNT or LNnT or DFL or SFL.

In one method according to the present invention, the oligosaccharide produced by the cell is an oligosaccharide of at least five monosaccharide units. Preferably, the oligosaccharide is a human milk oligosaccharide (HMO) of five monosaccharide units selected from the group consisting of LNFP-I, LNFP-II, LNFP-III, LNFP-V, LNFP-VI, LST-a, LST-b and LST-c.

In one method according to the present invention, the oligosaccharide produced by the cell is an oligosaccharide of at least six monosaccharide units. Preferably, the oligosaccharide is a human milk oligosaccharide (HMO) of six monosaccharide units selected from the group consisting of LNH, LNnH, pLNnH, pLNH-I, DSLNT, LNDFH-I, LNDFH-II and LNDFH-III.

In one aspect, the method according to the present invention produces a human milk oligosaccharide (HMO) of seven or eight monosaccharide units, such as an HMO selected from the group consisting of F-para-LNH I, FDS-LNH, TF-LNH, DS-LNH, F-LNH-II, DF-LNH-II, F-LNH-I, DF-LNH I, F-LNH-III and DF-LNH III. Production of these HMO's may require the presence of three or more glycosyltransferase activities.

### An Acceptor Oligosaccharide of at Least Three or Four or Five Monosaccharide Units

The genetically modified cell according to the present invention comprises a recombinant nucleic acid sequence and/or a cluster of recombinant nucleic acid sequences encoding a recombinant transporter protein and/or a cluster of recombinant proteins exemplary as shown in table 1 and in table 2, which are capable of importing an acceptor oligosaccharide of at least three, such as of at least four or five monosaccharide units into said cell. Typically, the acceptor oligosaccharide does not have more than 5 monosaccharide units.

In the context of the present invention an acceptor oligosaccharide is an oligosaccharide that can act as a substrate for a glycosyltransferase capable of transferring a glycosyl moiety from a glycosyl donor to the acceptor oligosaccharide. The glycosyl donor is preferably a nucleotide-activated sugar as described in the section on “glycosyltransferases”. Preferably, the acceptor oligosaccharide is a precursor for making a more complex HMO and can also be termed the precursor molecule.

The acceptor oligosaccharide containing at least three monosaccharide units can be either an end-product of a separate fermentation process employing a separate genetically modified cell, or an enzymatically or chemically produced molecule. In the former case, the acceptor oligosaccharide molecule can be isolated from a fermenter and subsequently added to the cultivation medium of another fermenter containing the cell that expresses a suitable oligosaccharide importer.

In one embodiment of the method, the acceptor oligosaccharide having at least three or four monosaccharide units is a neutral oligosaccharide. In one embodiment, it does not contain a fucosyl unit. In the present context, said acceptor oligosaccharide is preferably selected from the group consisting of LNT-II, LNT and LNnT.

Typically, the non-fucosylated acceptor oligosaccharide having at least three monosaccharide units is LNT-II and the acceptor oligosaccharide having at least four monosaccharide units is LNT or LNnT. This is in particular relevant when the transporter protein is a mutated lactose permease.

In another embodiment the acceptor oligosaccharide having at least three or four monosaccharide units is a neutral oligosaccharide comprising a fucosyl unit. In the present context, said acceptor oligosaccharide is preferably selected from the group consisting of 2′FL, 3FL and LNFP-I.

Typically, the fucosylated acceptor oligosaccharide having at least three monosaccharide units is 2′FL or 3FL and the and the acceptor oligosaccharide having at least four monosaccharide units is DFL and the acceptor oligosaccharide having at least five monosaccharide units is LNFP-I.

In embodiments the acceptor oligosaccharide of at least three monosaccharide units is supplied to the culture medium comprising a genetically engineered cell described herein. The acceptor oligosaccharide can for example be supplied to the culture by exogenously added it to the culture medium and/or it can be produced by microbial fermentation. In some embodiments the acceptor oligosaccharide is supplied by microbial fermentation of a second genetically modified cell, for example as described in WO 2022/242860 or EP application No. EP22209673.7.

### Glycosyltransferases

The genetically modified cell according to the present invention further comprises at least one recombinant nucleic acid sequence encoding at least one glycosyltransferase capable of transferring a glycosyl residue from a glycosyl donor to said acceptor oligosaccharide to synthesize a human milk oligosaccharide product having at least four monosaccharide units.

The glycosyltransferase is typically selected from the any one of the glycosyltransferases listed in tables 3, 6, 9 and 12.

The glycosyltransferase is preferably selected from the group consisting of fucosyltransferases, galactosyltransferases, glucosaminyltransferases, sialic acid transferases, N-acetylglucosaminyl transferases and N-acetylglucosaminyl transferases. In one aspect, the glycosyltransferase is selected from the beta-1,4-galactosyltransferases or beta-1,3-galactosyltransferases listed in tables 3, 6, 9 or 12.

Typically, the glycosyl donor is a nucleotide-activated sugar or an oligosaccharide, such as selected from the group consisting of glucose-UDP-GlcNac, GDP-fucose, UDP-galactose, UDP-glucose, UDP-N-acetylglucosamine, UDP-N-acetylgalactosamine and CMP-N-acetylneuraminic acid, preferably glucose-UDP-Gal and/or glucose-UDP-GlcNac.

Said glycosyl donor is synthesized by one or more genetically engineered cells and/or is exogenously added to the culture medium. Preferably, the glycosyl donor is a nucleotide-activated sugar which is synthesized by the host cell either using an already existing pathway, which may be modified to increase the pool of the relevant nucleotide-activated sugar or by introducing nucleotide sequences encoding for enzymes needed to produce the relevant nucleotide-activated sugar within the cell.

In the present invention, the at least one functional enzyme capable of transferring a saccharide moiety from a glycosyl donor to an acceptor oligosaccharide can be selected from the list consisting of galT and galTK. These enzymes can for example be used to produce LNnT or LNT, respectively, starting from LNT-II as acceptor oligosaccharide.

In a preferred embodiment the genetically modified cell according to the present invention does not comprise more than two recombinant nucleic acid sequences encoding a glycosyltransferase capable of transferring a glycosyl residue from a glycosyl donor to said acceptor oligosaccharide to synthesize a human milk oligosaccharide product having at least four monosaccharide units. The one or two glycosyltransferase activities are preferably selected from the activities described below.

### Heterologous β-1,3-N-acetyl-glucosaminyl-transferase

A heterologous β-1,3-N-acetyl-glucosaminyl-transferase is any protein which comprises the ability of transferring the N-acetyl-glucosamine of UDP-N-acetyl-glucosamine to lactose or another acceptor molecule, in a beta-1,3-linkage. A β-1,3-N-acetyl-glucosaminyl-transferase used herein does not originate in the species of the genetically engineered cell i.e. the gene encoding the β-1,3-galactosyltransferase is of heterologous origin. In the context of the resent invention the acceptor molecule, is an acceptor oligosaccharide of at least three or four monosaccharide units, e.g., LNT or LnNT, or more complex HMO structures Some of the examples below use the heterologous β-1,3-N-acetyl-glucosaminyl-transferase named LgtA or a variant thereof. In one example LgtA is used in combination with for example galT or galTK to produce pLNH or pLNnH, starting from LNnT as acceptor oligosaccharide.

### Heterologous 3-1,6-N-acetylglucosaminyltransferase

A heterologous β-1,6-N-acetyl-glucosaminyl-transferase is any protein which comprises the ability of transferring the N-acetyl-glucosamine of UDP-N-acetyl-glucosamine to an acceptor molecule, in a beta-1,6-linkage. A β-1,6-N-acetyl-glucosaminyl-transferase used herein does not originate in the species of the genetically engineered cell i.e., the gene encoding the β-1,6-galactosyltransferase is of heterologous origin. In the context of the resent invention the acceptor molecule, is an acceptor oligosaccharide of at least three or four monosaccharide units, e.g., LNT or LNnT, or more complex HMO structures Some of the examples below use the heterologous β-1,6-N-acetyl-glucosaminyl-transferase named Csp2, or a variant thereof to produce for example LNH or LNnH.

### Heterologous β-1,3-galactosyltransferase

A heterologous β-1,3-Galactosyltransferase is any protein that comprises the ability of transferring the galactose of UDP-Galactose to a N-acetyl-glucosaminyl moiety to an acceptor molecule in a beta-1,3-linkage. A β-1,3-galactosyltransferase used herein does not originate in the species of the genetically engineered cell i.e., the gene encoding the β-1,3-galactosyltransferase is of heterologous origin. In the context of the resent invention the acceptor molecule, is an acceptor oligosaccharide of at least three or four monosaccharide units, e.g., LNT-II, or more complex HMO structures. The examples below use the heterologous β-1,3-galactosyltransferase named GalTK or a variant thereof, to produce for example LNT or more complex HMOs in combination with other glycosyl transferases.

### Heterologous 3-1,4-galactosyltransferase

A heterologous β-1,4-Galactosyltransferase is any protein that comprises the ability of transferring the galactose of UDP-Galactose to a N-acetyl-glucosaminyl moiety. A β-1,4-galactosyltransferase used herein does not originate in the species of the genetically engineered cell i.e., the gene encoding the β-1,4-galactosyltransferase is of heterologous origin. In the context of the resent invention the acceptor molecule, is an acceptor oligosaccharide of at least three or four monosaccharide units, e.g., LNT-II, or more complex HMO structures. The examples below use the heterologous β-1,4-galactosyltransferase named GalT or a variant thereof, to produce for example LNnT or more complex HMOs in combination with other glycosyl transferases.

### Heterologous alpha-1, 2-fucosyltransferase

A heterologous alpha-1, 2-fucosyltransferase is a protein that comprises the ability to catalyze the transfer of fucose from a donor substrate, for example, GDP-fucose, to an acceptor molecule in an alpha-1,2-linkage. Preferably, an alpha-1, 2-fucosyltransferase used herein does not originate in the species of the genetically engineered cell i.e., the gene encoding the alpha-1, 2-fucosyltransferase is of heterologous origin. In the context of the present invention the acceptor molecule, is an acceptor oligosaccharide of at least three or four monosaccharide units, e.g., 2′-fucosyllactose, 3-fucosyllactose, LNT, LNFP-I or more complex HMO structures. The examples below use the heterologous alpha-1, 2-fucosyltransferase named FutC, Smob or FucT2, or a variant thereof, to produce for example DFL or LNFP-I or LNDFH-I.

### Heterologous alpha-1, 3-fucosyltranferase

A heterologous alpha-1, 3-fucosyltranferase refer to a glycosyltransferase that catalyzes the transfer of fucose from a donor substrate for example, GDP-fucose, to an acceptor molecule in an alpha-1,3-linkage. Preferably, an alpha-1, 3-fucosyltransferase used herein does not originate in the species of the genetically engineered cell i.e., the gene encoding the alpha-1, 3-fucosyltransferase is of heterologous origin. In the context of the resent invention the acceptor molecule, is an acceptor oligosaccharide of at least three or four monosaccharide units, e.g., 2′-fucosyllactose, 3-fucosyllactose, LNT, LNFP-I or more complex HMO structures. The examples below use the heterologous alpha-1, 2-fucosyltransferase named FutA or FucT, or a variant thereof, to produce for example DFL, LNFP-III, LNFP-V, LNDFH-II or LNDFH-III.

### Heterologous alpha-1,3/4-fucosyltransferase

A heterologous alpha-1,3/4-fucosyltransferase refer to a glycosyltransferase that catalyzes the transfer of fucose from a donor substrate for example, GDP-fucose, to an acceptor molecule in an alpha-1,3- or alpha 1,4-linkage. Preferably, an alpha-1,3/4-fucosyltransferase used herein does not originate in the species of the genetically engineered cell i.e., the gene encoding the alpha-1,3/4-fucosyltransferase is of heterologous origin. In the context of the resent invention the acceptor molecule, is an acceptor oligosaccharide of at least three or four monosaccharide units, e.g., 2′-fucosyllactose, 3-fucosyllactose, LNT, LNFP-I or more complex HMO structures. The examples below use the heterologous alpha-1,3/4-fucosyltransferase named FucTIII, or a variant thereof, to produce for example LNFP-I, LNFP-I, LNFP-V, LNDFH-I or LNDFH-II.

### Heterologous alpha-2,3-sialyltransferase

A heterologous alpha-2,3-sialyltransferase refer to a glycosyltransferase that catalyzes the transfer of sialyl from a donor substrate for example, CMP-N-acetylneuraminic acid, to an acceptor molecule in an alpha-2,3-linkage. Preferably, an alpha-2,3-sialyltransferase used herein does not originate in the species of the genetically engineered cell i.e., the gene encoding the 2,3-sialyltransferase is of heterologous origin. In the context of the resent invention the acceptor molecule, is an acceptor oligosaccharide of at least three or four monosaccharide units, e.g., 3-fucosyllactose, LNT, LNnT or more complex HMO structures. The examples below use the heterologous 2,3-sialyltransferase named PM70 or Ccol or a variant thereof, to produce for example Lst-a or if combined with an alpha-2,6-sialyltransferase to produce DSLNT.

### Heterologous alpha-2,6-sialyltransferase

A heterologous alpha-2,6-sialyltransferase refer to a glycosyltransferase that catalyzes the transfer of sialyl from a donor substrate for example, CMP-N-acetylneuraminic acid, to an acceptor molecule in an alpha-2,6-linkage. Preferably, an alpha-2,6-sialyltransferase used herein does not originate in the species of the genetically engineered cell i.e., the gene encoding the 2,6-sialyltransferase is of heterologous origin. In the context of the resent invention the acceptor molecule, is an acceptor oligosaccharide of at least three or four monosaccharide units, e.g., 3-fucosyllactose, LNT, LNnT or more complex HMO structures. The examples below use the heterologous 2,3-sialyltransferase named Pst6 or HAC1266 or a variant thereof, to produce e.g., Lst-b, Lst-c or if combined with an alpha-2,3-sialyltransferase to produce DSLNT.

### Pathways to Produce Nucleotide-Activated Sugar

When carrying out the method of this invention, a glycosyltransferase mediated glycosylation reaction preferably takes place in which an activated sugar nucleotide serves as donor. An activated sugar nucleotide generally has a phosphorylated glycosyl residue attached to a nucleoside, a specific glycosyl transferase enzyme accepts only a specific sugar nucleotide. Thus, preferably the following activated sugar nucleotides are involved in the glycosyl transfer: glucose-UDP-GlcNAc, GDP-fucose, UDP-galactose, UDP-glucose, UDP-N-acetylglucosamine, UDP-N-acetylgalactosamine (GlcNAc) and CMP-N-acetylneuraminic acid. The genetically modified cell according to the present invention can comprise one or more pathways to produce a nucleotide-activated sugar selected from the group consisting of glucose-UDP-GlcNAc, GDP-fucose, UDP-galactose, UDP-glucose, UDP-N-acetylglucosamine, UDP-N-acetylgalactosamine and CMP-N-acetylneuraminic acid.

In one embodiment of the method, the genetically modified cell is capable of producing one or more activated sugar nucleotide mentioned above by a de novo pathway. In this regard, an activated sugar nucleotide is made by the cell under the action of enzymes involved in the de novo biosynthetic pathway of that respective sugar nucleotide in a stepwise reaction sequence starting from a simple carbon source like glycerol, fructose or glucose (for a review for monosaccharide metabolism see e.g. H. H. Freeze and A. D. Elbein: Chapter 4: Glycosylation precursors, in: Essentials of Glycobiology, 2nd edition (Eds. A. Varki et al.), Cold Spring Harbour Laboratory Press (2009)).The enzymes involved in the de novo biosynthetic pathway of an activated sugar nucleotide can be naturally present in the cell or introduced into the cell by means of gene technology or recombinant DNA techniques, all of them are parts of the general knowledge of the skilled person.

In another embodiment, the genetically modified cell can utilize salvaged monosaccharide for producing activated sugar nucleotide. In the salvage pathway, monosaccharides derived from degraded oligosaccharides are phosphorylated by kinases, and converted to nucleotide sugars by pyrophosphorylases. The enzymes involved in the procedure can be heterologous ones, or native ones of the cell used for genetic modification.

### Major Facilitator Superfamily Transporter Proteins

The oligosaccharide as product can be accumulated both in the intra- and the extracellular matrix. The product can be transported to the supernatant in a passive way, i.e., it diffuses outside across the cell membrane. Alternatively, the HMO transport can be facilitated by major facilitator superfamily transporter proteins that promote the effluence of sugar derivatives from the cell to the supernatant. The major facilitator superfamily transporter can be present exogenously or endogenously and is overexpressed under the conditions of the fermentation to enhance the export of the oligosaccharide derivative produced. The specificity towards the sugar moiety of the product to be secreted can be altered by mutation by means of known recombinant DNA techniques.

Thus, the genetically modified cell according to the present invention can further comprise a nucleic acid sequence encoding a major facilitator superfamily transporter protein capable of exporting the human milk oligosaccharide product having at least four monosaccharide units into the extracellular medium.

Over the past decade several new and efficient major facilitator superfamily transporter proteins have been identified, each having specificity for different recombinantly produced HMOs and development of recombinant cells expressing said proteins are advantageous for high scale industrial HMO manufacturing. Sugar transport relates to the transport of a sugar, such as, but not limited to, an oligosaccharide, such as an HMO.

Thus, in one or more exemplary embodiments, the genetically engineered cell according to the method described herein further comprises a gene product that acts as a major facilitator superfamily transporter. The gene product that acts as a major facilitator superfamily transporter may be encoded by a recombinant nucleic acid sequence that is expressed in the genetically engineered cell. The recombinant nucleic acid sequence encoding a major facilitator superfamily transporter, may be integrated into the genome of the genetically engineered cell.

In one embodiment, the genetically modified cell of the invention comprises a nucleic acid sequence encoding a major facilitator superfamily transporter protein capable of exporting the human milk oligosaccharide product having at least four monosaccharide units into the extracellular medium.

### Vag

In one embodiment, the genetically modified cell of the invention comprises a nucleic acid sequence encoding an efflux transporter protein capable of exporting the human milk oligosaccharide product having at least four monosaccharide units, such as DFL, LNT or LNnT, into the extracellular medium, which is a heterologous gene encoding a putative MFS (major facilitator superfamily) transporter protein, originating from the bacterium Pantoea vagans. More specifically, the invention relates to a genetically modified cell optimized to produce an oligosaccharide, in particular an HMO, comprising a recombinant nucleic acid encoding a protein having at least 80%, such as 85%, such as 90% such as 95% or 100% sequence identity to the amino acid sequence of the amino acid sequence having the GenBank accession ID WP_048785139.1 (https://www.ncbi.nlm.nih.gov/protein/WP_048785139.1). Said MFS transporter protein is further described in WO2021148611 and is identified herein as “Vag protein” or “Vag transporter” or “Vag”, interchangeably; a nucleic acid sequence encoding Vag protein is identified herein as “vag coding nucleic acid/DNA” or “vag gene” or “vag”.

Vag facilitates an increase in the efflux of the produced HMOs of at least four units of monosaccharides, e.g., difucosyllactose (DFL), lacto-N-neotetraose (LNnT) and lacto-N-tetraose (LNT). Further, the total production of the HMOs lacto-N-neotetraose (LNnT) and lacto-N-tetraose (LNT) by the corresponding HMO-producing cells expressing Vag is also increased, while the by-product formation, e.g., para-lacto-N-neohexaose (pLNnH) and para-lacto-N-hexaose II (pLNH-II) in these cells, correspondingly, is often decreased and said by-product oligosaccharides typically accumulate in the cell interior of the HMO production systems. Further, expression of the Vag protein in HMO-producing cells leads to a reduction in biomass formation during high-cell density fermentations and to healthier cell cultures, as it is e.g., reflected by a decrease in the number of dead cells at the end of fermentation, which makes the manufacturing process more efficient as more product is produced per biomass unit.

### LacY Negative

In one aspect, the genetically modified cell according to the present invention does not express a functional lactose importer, such as a lactose permease. In embodiments the genetically modified cell is lacY negative. In particular the genetically modified cell does not express the wild-type lactose permease, but may express the lactose permease mutants in table 2.

The E. coli endogenous native lactose permease (LacY) has specificity towards galactose and simple galactosyl disaccharides like lactose. The disruption of the endogenous native lacY gene in E. coli is thus a highly sufficient genetic tool to specifically hinder the import of lactose from the cell exterior into the cytoplasm and thus for ensuring that preferably oligosaccharides with a more complex structure, such as oligosaccharides of at least 3, such as at least 4, monosaccharide units are imported into said cell by means of the herein described specific transporter protein and/or a cluster of proteins capable of importing an acceptor oligosaccharide of at least 3, such as at least 4, monosaccharide units into said cell.

In the present context, the term “lacY negative” or lactose permease negative” is used to describe the disruption of the native lactose permease (e.g., LacY) in the genetically modified cell and does not exclude that the genetically modified cell comprises a recombinant nucleic acid sequence that is selected from the group consisting of mutated LacY (e.g., as shown in table 2), as long as that recombinant nucleic acid sequence encodes a transporter protein and/or a cluster of proteins capable of importing an acceptor oligosaccharide of at least 3, such as at least 4, monosaccharide units into said cell.

### The Genetically Modified Cell

In the present context, the terms “a genetically modified cell” and “a genetically engineered cell” are used interchangeably. As used herein “a genetically modified cell” is a cell whose genetic material has been altered by human intervention using a genetic engineering technique, such a technique is for example but not limited to transformation or transfection e.g., with a heterologous polynucleotide sequence, Crisper/Cas editing and/or random mutagenesis. In one embodiment the genetically engineered cell has been transformed or transfected with a recombinant nucleic acid sequence.

The genetically engineered cell is preferably a prokaryotic cell, such as a microbial cell. Appropriate microbial cells that may function as a host cell include yeast cells, bacterial cells, archaebacterial cells, algae cells, and fungal cells.

The genetically engineered cell (host cell) may be e.g., a bacterial or yeast cell. In one preferred embodiment, the genetically engineered cell is a bacterial cell.

### Host Cells

Regarding the bacterial host cells, there are, in principle, no limitations; they may be eubacteria (gram-positive or gram-negative) or archaebacteria, as long as they allow genetic manipulation for insertion of a gene of interest and can be cultivated on a manufacturing scale. Preferably, the host cell has the property to allow cultivation to high cell densities. Non-limiting examples of bacterial host cells that are suitable for recombinant industrial production of an HMO(s) according to the invention could be Erwinia herbicola (Pantoea agglomerans), Citrobacter freundii, Pantoea citrea, Pectobacterium carotovorum, or Xanthomonas campestris. Bacteria of the genus Bacillus may also be used, including Bacillus subtilis, Bacillus licheniformis, Bacillus coagulans, Bacillus thermophilus, Bacillus laterosporus, Bacillus megaterium, Bacillus mycoides, Bacillus pumilus, Bacillus lentus, Bacillus cereus, and Bacillus circulans. Similarly, bacteria of the genera Lactobacillus and Lactococcus may be engineered using the methods of this invention, including but not limited to Lactobacillus acidophilus, Lactobacillus salivarius, Lactobacillus plantarum, Lactobacillus helveticus, Lactobacillus delbrueckii, Lactobacillus rhamnosus, Lactobacillus bulgaricus, Lactobacillus crispatus, Lactobacillus gasseri, Lactobacillus casei, Lactobacillus reuteri, Lactobacillus jensenii, and Lactococcus lactis. Corynebacterium glutamicum, Streptococcus thermophiles and Proprionibacterium freudenreichii are also suitable bacterial species for the invention described herein. Also included as part of this invention are strains, engineered as described here, from the genera Enterococcus (e.g., Enterococcus faecium and Enterococcus thermophiles), Bifidobacterium (e.g., Bifidobacterium longum, Bifidobacterium infantis, and Bifidobacterium bifidum), Sporolactobacillus spp., Micromomospora spp., Micrococcus spp., Rhodococcus spp., and Pseudomonas (e.g., Pseudomonas fluorescens and Pseudomonas aeruginosa).

In one or more exemplary embodiments, the genetically engineered cell is selected from the group consisting of wherein said modified cell is selected from the group consisting of Escherichia coli, Corynebacterium glutamicum, Lactococcus lactis, Bacillus subtilis, Streptomyces lividans, Pichia pastoris and Saccharomyces cerevisiae.

In one or more exemplary embodiments, the genetically engineered cell is B. subtilis.

The genetically modified cell according to the present invention is preferably a microbial cell, such as a gram-negative bacterium or a fungus, such as a fungi selected from a yeast cells of the genera Komagataella, Kluyveromyces, Yarrowia, Pichia, Saccaromyces, Schizosaccharomyces or Hansenula or from a filamentous fungi of the genera Aspargillus, Fusarium or Thricoderma, or a gram-negative bacterium selected from the group consisting of Escherichia sp., and Campylobacter sp.

In one or more exemplary embodiments, the genetically engineered cell is S. cerevisiae or P. pastoris.

In one or more exemplary embodiments, the genetically engineered cell is Escherichia coli.

In one or more exemplary embodiments, the invention relates to a genetically engineered cell, wherein the cell is derived from the E. coli K-12 strain or DE3.

### A Recombinant Nucleic Acid Sequence

The present invention relates to a genetically modified cell comprising a recombinant nucleic acid sequence and/or a cluster of recombinant nucleic acid sequences encoding a recombinant transporter protein and/or a cluster of recombinant proteins capable of importing an acceptor oligosaccharide of at least 3, such as at least 4, monosaccharide units into said cell.

In the present context, the term “recombinant nucleic acid sequence”, “recombinant gene/nucleic acid/nucleotide sequence/DNA encoding” or “coding nucleic acid sequence” is used interchangeably and intended to mean an artificial nucleic acid sequence (i.e. produced in vitro using standard laboratory methods for making nucleic acid sequences) that comprises a set of consecutive, non-overlapping triplets (codons) which is transcribed into mRNA and translated into a protein when under the control of the appropriate control sequences, i.e. a promoter sequence. It is understood that a recombinant nucleic acid sequence inserted into a cell is non-identical to endogenous nucleic acid sequence. It may for example be a variant of an endogenous nucleic acid sequence or it may be an additional copy of an endogenous nucleic acid sequence under control of a different promoter than the endogenous equivalent of the recombinant nucleic acid sequence. A protein encoded by a recombinant nucleic acid sequence, where the protein is distinguishable from the endogenous protein is also considered a recombinant protein.

The boundaries of the coding sequence are generally determined by a ribosome binding site located just upstream of the open reading frame at the 5′end of the mRNA, a transcriptional start codon (AUG, GUG or UUG), and a translational stop codon (UAA, UGA or UAG). A coding sequence can include, but is not limited to, genomic DNA, cDNA, synthetic, and recombinant nucleic acid sequences.

The term “nucleic acid” includes RNA, DNA and cDNA molecules. It is understood that, as a result of the degeneracy of the genetic code, a multitude of nucleic acid sequences encoding a given protein may be produced.

The recombinant nucleic acid sequence may be a coding DNA sequence e.g., a gene, or non-coding DNA sequence e.g., a regulatory DNA, such as a promoter sequence.

The recombinant nucleic acid sequence may in addition be heterologous. As used herein “heterologous” refers to a polypeptide, amino acid sequence, nucleic acid sequence or nucleotide sequence that is foreign to a cell or organism, i.e., to a polypeptide, amino acid sequence, nucleic acid molecule or nucleotide sequence that does not naturally occurs in said cell or organism.

The invention also relates to a nucleic acid construct comprising a coding nucleic sequence, i.e. recombinant DNA sequence of a gene of interest, e.g., a fucosyltransferase gene, and a non-coding regulatory DNA sequence, e.g., a promoter DNA sequence, e.g., a recombinant promoter sequence derived from the promoter sequence of lac operon or an glp operon, such as a glpF promoter as described in WO2019/123324 and WO2020/255054, or a promoter sequence derived from another genomic promoter DNA sequence, or a synthetic promoter sequence, wherein the coding and promoter sequences are operably linked.

In exemplary embodiments the promoter is a constitutive or inducible promoter, and may for example be selected from PglpF (SEQ ID NO: 70), Plac (SEQ ID NO: 71), PtacI (SEQ ID NO: 72), PtacII (SEQ ID NO: 73), PosmY (SEQ ID NO: 74) or Pbad (SEQ ID NO: 75).

The term “operably linked” refers to a functional relationship between two or more nucleic acid (e.g., DNA) segments. operably linked refers to the functional relationship of a transcriptional regulatory sequence to a transcribed sequence. For example, a promoter sequence is operably linked to a coding sequence if it stimulates or modulates the transcription of the coding sequence in an appropriate host cell or other expression system. In one embodiment of the invention the cluster of recombinant nucleic acid sequences encoding a transporter protein are operably linked such that they are transcribed by a single promoter sequence.

Generally, promoter sequences that are operably linked to a transcribed sequence are physically contiguous to the transcribed sequence, i.e., they are cis-acting.

In one exemplified embodiment, the nucleic acid construct of the invention may be a part of the vector DNA, in another embodiment, the construct it is an expression cassette/cartridge that is integrated in the genome of a host cell.

Accordingly, the term “nucleic acid construct” means an artificially constructed segment of nucleic acids, in particular a DNA segment, which is intended to be inserted into a target cell, e.g., a bacterial cell, to modify expression of a gene of the genome or express a gene/coding DNA sequence which may be included in the construct.

Integration of the nucleic acid construct of interest comprised in the construct (expression cassette) into the bacterial genome can be achieved by conventional methods, e.g. by using linear cartridges that contain flanking sequences homologous to a specific site on the chromosome, as described for the attTn7-site (Waddell C. S. and Craig N. L., Genes Dev. (1988) February; 2(2):137-49.); methods for genomic integration of nucleic acid sequences in which recombination is mediated by the Red recombinase function of the phage A or the RecE/RecT recombinase function of the Rac prophage (Murphy, J Bacteriol. (1998); 180(8):2063-7; Zhang et al., Nature Genetics (1998) 20: 123-128 Muyrers et al., EMBO Rep. (2000) 1(3): 239-243); methods based on Red/ET recombination (Wenzel et al., Chem Biol. (2005), 12(3):349-56.; Vetcher et al., Appl Environ Microbiol. (2005); 71(4):1829-35); or positive clones, i.e., clones that carry the expression cassette, can be selected e.g., by means of a marker gene, or loss or gain of gene function.

In one or more exemplary embodiments, the present disclosure relates to one or more of the recombinant nucleic acid sequences as illustrated in SEQ ID NO: 52, 53, 54, 55, 56, 57, 58, 59, 60, 61, 62, 63, 64, 65, 66, 67, 68, or 69, encoding an ABC importer protein complex or a MFS importer protein.

In particular, the present disclosure relates to one or more of a recombinant nucleic acid sequence and/or to a functional homologue thereof having a sequence which is at least 70% identical to SEQ ID NO: 52, 53, 54, 55, 56, 57, 58, 59, 60, 61, 62, 63, 64, 65, 66, 67, 68, or 69, such as at least 75% identical, at least 80% identical, at least 85% identical, at least 90% identical, at least, at least 95% identical, at least 98% identical, or 100% identical.

### Sequence Identity

The term “sequence identity” as used herein describes the relatedness between two amino acid sequences or between two nucleotide sequences, i.e., a candidate sequence (e.g., a sequence of the invention) and a reference sequence (such as a prior art sequence) based on their pairwise alignment. For purposes of the present invention, the sequence identity between two amino acid sequences is determined using the Needleman-Wunsch algorithm (Needleman and Wunsch, 1970, J. Mo/. Biol. 48: 443-453) as implemented in the Needle program of the EMBOSS package (EMBOSS: The European Molecular Biology Open Software Suite, Rice et al., 2000, Trends Genet. 16: 276-277,), preferably version 5.0.0 or later (available at https://www.ebi.ac.uk/Tools/psa/emboss needle/). The parameters used are gap open penalty of 10, gap extension penalty of 0.5, and the EBLOSUM62 (EMBOSS version of 30 BLOSUM62) substitution matrix. The output of Needle labeled “longest identity” (obtained using the −nobrief option) is used as the percent identity and is calculated as follows: (Identical Residues×100)/(Length of Alignment−Total Number of Gaps in Alignment)

For purposes of the present invention, the sequence identity between two nucleotide sequences is determined using the Needleman-Wunsch algorithm (Needleman and Wunsch, 1 970, supra) as implemented in the Needle program of the EMBOSS package (EMBOSS: The European Molecular Biology Open Software Suite, Rice et al., 2000, Trends Genet. 16: 276-277), 10 preferably version 5.0.0 or later. The parameters used are gap open penalty of 10, gap extension penalty of 0.5, and the DNAFULL (EMBOSS version of NCBI NUC4.4) substitution matrix. The output of Needle labeled “longest identity” (obtained using the −nobrief option) is used as the percent identity and is calculated as follows: (Identical Deoxyribonucleotides×100)/(Length of Alignment−Total Number of Gaps in Alignment).

### Functional Homologue

A functional homologue or functional variant of a protein/nucleic acid sequence as described herein is a protein/nucleic acid sequence with alterations in the genetic code, which retain its original functionality. A functional homologue may be obtained by mutagenesis or may be natural occurring variants from the same or other species. The functional homologue should have a remaining functionality of at least 50%, such as 60%, 70%, 80%, 90% or 100% compared to the functionality of the protein/nucleic acid sequence.

A functional homologue of any one of the disclosed amino acid or nucleic acid sequences can also have a higher functionality. A functional homologue of any one of the amino acid sequences shown in any of tables 1-12 or a recombinant nucleic acid encoding these sequences or as disclosed in SE ID NO: 52, 53, 54, 55, 56, 57, 58, 59, 60, 61, 62, 63, 64, 65, 66, 67, 68, or 69, should ideally be able to participate in the HMO production, in terms of increased HMO yield, export of HMO product out of the cell or import of substrate for the HMO production, such as a acceptor oligosaccharide of at least three monosaccharide units, improved purity/by-product formation, reduction in biomass formation, viability of the genetically engineered cell, robustness of the genetically engineered cell according to the disclosure, or reduction in consumables needed for the production.

### Use of a Genetically Modified Cell

The disclosure also relates to any commercial use of the genetically modified cell(s) or the nucleic acid construct(s) disclosed herein, such as, but not limited to, in a method for producing a human milk oligosaccharide (HMO) having at least four monosaccharide units.

In an exemplified embodiment, the genetically modified cell and/or the nucleic acid construct according to the invention, is used in the manufacturing of one or more HMO(s), wherein the HMOs are LNT or LNnT.

In one or more exemplary embodiments, the genetically engineered cell and/or the nucleic acid construct is used in the manufacturing of LNT.

In one or more exemplary embodiments, the genetically engineered cell and/or the nucleic acid construct is used in the manufacturing of LNnT.

In one or more exemplary embodiments, the genetically engineered cell and/or the nucleic acid construct is used in the manufacturing of a neutral non-fucosylated HMO selected from the group consisting of LNT, LNnT, LNH, pLNnH, LNnH and pLNH-l.

In one or more exemplary embodiments, the genetically engineered cell and/or the nucleic acid construct is used in the manufacturing of a fucosylated HMO selected from the group consisting of DFL, FSL, LNFP-I, LNFP-II, LNFP-III, LNFP-V, LNFP-VI, LNDFH-1, LNDFH-II and LNDFH-III.

In one or more exemplary embodiments, the genetically engineered cell and/or the nucleic acid construct is used in the manufacturing of a sialylated HMO selected from the group consisting of FSL, DSLNT, LST-a, LST-b and LST-c.

In one or more exemplary embodiments, the genetically engineered cell and/or the nucleic acid construct is used in the manufacturing of an HMO consisting of four monosaccharide units, such as an HMO selected from the group consisting of LNT, LNnT, DFL and SFL,

In one or more exemplary embodiments, the genetically engineered cell and/or the nucleic acid construct is used in the manufacturing of an HMO consisting of five monosaccharide units, such as an HMO selected from the group consisting of LNFP-I, LNFP-II, LNFP-III, LNFP-V, LNFP-VI, DSLNT, LST-a, LST-b and LST-c

In one or more exemplary embodiments, the genetically engineered cell and/or the nucleic acid construct is used in the manufacturing of an HMO consisting of six monosaccharide units, such as an HMO selected from the group consisting of DSLNT, pLNnH, pLNH-I, LNH, LNnH, LNDFH-I, LNDFH-II and LNDFH-III.

In one or more exemplary embodiments, the genetically engineered cell and/or the nucleic acid construct may be modified with additional glycosyltransferase activities to produces a human milk oligosaccharide (HMO) of seven or eight monosaccharide units, such as an HMO selected from the group consisting F-para-LNH I, FDS-LNH, TF-LNH, DS-LNH, F-LNH-II, DF-LNH-II, F-LNH-I, DF-LNH I, F-LNH-III, DF-LNH III. Production of these HMO's may require the presence of three or more glycosyltransferase activities.

### A Method for Producing a Human Milk Oligosaccharide (HMO) Having at Least Four Monosaccharide Units

The present invention also relates to a method for producing a oligosaccharide having at least four monosaccharide units, such as a human milk oligosaccharide (HMO), said method comprising culturing a genetically modified cell according to the present invention.

The present invention thus relates to a method for producing a oligosaccharide, such as a human milk oligosaccharide (HMO) having at least four monosaccharide units, said method comprising culturing a genetically modified cell comprising:
a recombinant nucleic acid sequence and/or a cluster of recombinant nucleic acid sequences encoding a transporter protein and/or a cluster of proteins capable of importing an acceptor oligosaccharide of at least three monosaccharide units into said cell,
at least one recombinant nucleic acid encoding at least one glycosyltransferase capable of transferring a glycosyl residue from a glycosyl donor to said acceptor oligosaccharide to synthesize a human milk oligosaccharide product having at least four monosaccharide units,
   wherein the recombinant nucleic acid sequence and/or the cluster of recombinant nucleic acid sequences encoding a transporter protein and/or a cluster of proteins capable of importing said acceptor oligosaccharide of at least three monosaccharide units is selected from the group consisting of mutated lactose permease shown in table 2 and ABC-importers or MFS importers from a gram-positive bacterium, as shown in table 1.

The method in particular comprises culturing a genetically modified cell comprising a cluster of recombinant nucleic acid sequences encoding a cluster of transporter proteins capable of importing said acceptor oligosaccharide of at least three monosaccharide units which is an ABC importer selected from the group consisting of:
a. Blon2177, 2176 and 2175 (TP8 in table 1) comprising three sub-units with the amino acid sequences comprising or consisting of SEQ ID NO: 13, 14 and 15 or amino acid sequences with at least 80%, such as 85%, such as 90%, such as 95% identity to SEQ ID: 13, 14 and 15;
b. RHOM_04095, 04100, 04105 (TP9 in table 1) comprising three sub-units with the amino acid sequences comprising or consisting of SEQ ID NO: 35, 36 and 37 or amino acid sequences with at least 80%, such as 85%, such as 90%, such as 95% identity to SEQ ID: 35, 36 and 37;
c. BBPC_1775, 1776, 1777 (TP18 in table 1) comprising three sub-units with the amino acid sequences comprising or consisting of SEQ ID NO: 47, 48 and 49 or amino acid sequences with at least 80%, such as 85%, such as 90%, such as 95% identity to SEQ ID: 47, 48 and 49;
d. Bbr_0527, 0528, 0530, 0531 (TP11 in table 1). comprising four sub-units with the amino acid sequences comprising or consisting of SEQ ID NO: 28, 29, 30 and 50 or amino acid sequences with at least 80%, such as 85%, such as 90%, such as 95% identity to SEQ ID: 28, 29, 30 and 50;
e. HMPREF0373_02960, 0373_02961, 0373_02962 (TP10 in table 1) comprising three sub-units with the amino acid sequences comprising or consisting of SEQ ID NO: 38, 39 and 40 or amino acid sequences with at least 80%, such as 85%, such as 90%, such as 95% identity to SEQ ID: 38, 39 and 40; and
f. BBKW_1838, 1839, 1840 (TP17 in table 1) comprising three sub-units with the amino acid sequences comprising or consisting of SEQ ID NO: 44, 45 and 46 or amino acid sequences with at least 80%, such as 85%, such as 90%, such as 95% identity to SEQ ID: 44, 45 and 46 or with the amino acid sequences comprising or consisting of SEQ ID NO: 41, 42 and 43 or amino acid sequences with at least 80%, such as 85%, such as 90%, such as 95% identity to SEQ ID: 41, 42 and 43.

The method in particular comprises culturing a genetically modified cell comprising a cluster of recombinant nucleic acid sequences encoding a cluster of transporter proteins capable of importing said acceptor oligosaccharide of at least three monosaccharide units which is an MFS importer selected from the group consisting of:
a. Blon:0247 (TP1 in table 1) comprising or consisting of an amino acid sequence of SEQ ID NO: 2 or an amino acid sequence with at least 80%, such as 85%, such as 90%, such as 95% identity to SEQ ID: 2;
b. Blon_0431 (TP2 in table 1 comprising or consisting of an amino acid sequence of SEQ ID NO: 6 or an amino acid sequence with at least 80%, such as 85%, such as 90%, such as 95% identity to SEQ ID: 6;
c. Blon_0788 (TP3 in table 1) comprising or consisting of an amino acid sequence of SEQ ID NO: 7 or an amino acid sequence with at least 80%, such as 85%, such as 90%, such as 95% identity to SEQ ID: 7;
d. Blon_0962 (TP13 in table 1) comprising or consisting of an amino acid sequence of SEQ ID NO: 12 or an amino acid sequence with at least 80%, such as 85%, such as 90%, such as 95% identity to SEQ ID: 12;
e. Blon_2307 (TP14 in table 1) comprising or consisting of an amino acid sequence of SEQ ID NO: 19 or an amino acid sequence with at least 80%, such as 85%, such as 90%, such as 95% identity to SEQ ID: 19; and
f. Blon_2400 (TP4 in table 1) comprising or consisting of an amino acid sequence of SEQ ID NO: 27 or an amino acid sequence with at least 80%, such as 85%, such as 90%, such as 95% identity to SEQ ID: 27

In one aspect, the method according to the present invention produces a human milk oligosaccharide (HMO) of only four monosaccharide units, such as DFL, FSL, LNT or LNnT.

In one aspect, the method according to the present invention produces a human milk oligosaccharide (HMO) of at least five monosaccharide units, such as an HMO selected from the list consisting of LNFP-I, LNFP-II, LNFP-III, LNFP-V, LNFP-VI, DSLNT, LST-a, LST-b and LST-c.

In one aspect, the method according to the present invention produces a human milk oligosaccharide (HMO) of six monosaccharide units, such as an HMO selected from the list consisting of DSLNT, pLNnH, pLNH-I, LNH, LNnH, LNDFH-I, LNDFH-II and LNDFH-III.

In one aspect, the method according to the present invention produces a human milk oligosaccharide (HMO) of seven or eight monosaccharide units, such as an HMO selected from the group consisting F-para-LNH I, FDS-LNH, TF-LNH, DS-LNH, F-LNH-II, DF-LNH-II, F-LNH-I, DF-LNH I, F-LNH-III, DF-LNH III. Production of these HMO's may require the presence of three or more glycosyltransferase activities.

The method of producing an oligosaccharide, such as a human milk oligosaccharide (HMO) having at least four monosaccharide units of the present invention further comprises providing a glycosyl donor, which is synthesized separately by one or more genetically engineered cells and/or is exogenously added to the culture medium from an alternative source.

The method of producing an oligosaccharide, such as a human milk oligosaccharide (HMO) having at least four monosaccharide unit of the present invention further comprises providing an acceptor oligosaccharide of at least three monosaccharide units, which is exogenously added to the culture medium and/or it can be produced by microbial fermentation. In some embodiments the acceptor oligosaccharide is supplied by microbial fermentation of a second genetically modified cell, for example as described in WO 2022/242860 or EP application No. EP22209673.7.

The oligosaccharide, such as the human milk oligosaccharide (HMO), having at least four monosaccharide units is preferably retrieved from the culture medium and/or the genetically modified cell.

In particular, the present invention relates to a method for producing LNnT, said method comprising:
a) obtaining a genetically modified cell comprising
   i. a nucleic acid sequence encoding a transporter protein capable of importing LNT-II, selected from the group consisting of a mutant lactose permease of table 2,
   ii. at least one nucleic acid sequence encoding a heterologous β-1,4-galactosyltransferase, such as galT, preferably under control of a PglpF promoter, and
   iii. optionally, a nucleic acid sequence encoding vag, preferably under control of a PglpF or Plac promoter, and
b) culturing said genetically modified cell in a carbon-source containing culture medium in the presence of LNT-II to be internalized into said genetically modified cell, and
c) to produce said human milk oligosaccharide (HMO) having at least four monosaccharide units, in particular LNnT, by said genetically modified cell, and
d) retrieving the human milk oligosaccharide (HMO) having at least four monosaccharide units, in particular LNnT, from the culture medium and/or the genetically modified cell.

In particular, the present invention relates to a method for producing LNT, said method comprising:
a) obtaining a genetically modified cell comprising
   i.a nucleic acid sequence encoding a transporter protein capable of importing LNT-II, selected from the group consisting of a mutant lactose permease of table 2,
   ii. at least one nucleic acid sequence encoding a heterologous β-1,3-galactosyltransferase, such as galTK, preferably under control of a PglpF promoter, and
   iii. optionally, a nucleic acid sequence encoding Vag, preferably under control of a PglpF or Plac promoter, and
b) culturing said genetically modified cell in a carbon-source containing culture medium in the presence of LNT-II to be internalized into said genetically modified cell, and
c) to produce said human milk oligosaccharide (HMO) having at least four monosaccharide units, in particular LNT, by said genetically modified cell, and
d) retrieving the human milk oligosaccharide (HMO) having at least four monosaccharide units, in particular LNT, from the culture medium and/or the genetically modified cell.

In particular, the present invention relates to a method for producing LNFP-I, said method comprising:
a) obtaining a genetically modified cell comprising
   i.a nucleic acid sequence encoding a transporter protein capable of importing LNT, selected from the group of ABC importers or MFS importers in Table 5, in particular, an importer selected from the group consisting of TP ID NO: 1, 2, 3, 8, 9, and 10, and
   ii. at least one nucleic acid sequence encoding a heterologous alpha-1,2-fucosylsyltransferase, such as FutC or Smob, preferably under control of a PglpF promoter, and
   iii. optionally, a nucleic acid sequence encoding vag, preferably under control of a PglpF or Plac promoter, and
b) culturing said genetically modified cell in a carbon-source containing culture medium in the presence of LNT to be internalized into said genetically modified cell, and
c) to produce said human milk oligosaccharide (HMO) having at least four monosaccharide units, in particular LNFP-I, by said genetically modified cell, and
d) retrieving the human milk oligosaccharide (HMO) having at least four monosaccharide units, in particular LNFP-I, from the culture medium and/or the genetically modified cell.

In particular, the present invention relates to a method for producing LNFP-I, LNFP-II, LNFP-V, LNDFH-1 or LNFDH-II, said method comprising:
a) obtaining a genetically modified cell comprising
   i. a nucleic acid sequence encoding a transporter protein capable of importing LNT, selected from the group of ABV importers or MFS importers in Table 5, in particular, an importer selected from the group consisting of TP ID NO: 1, 2, 3, 8, 9, and 10, and
   ii. at least one nucleic acid sequence encoding a fucosyltransferase, such as a heterologous alpha-1,2-fucosylsyltransferase, alpha-1,3-fucosylsyltransferase, or alpha-1,3/4-fucosylsyltransferase as listed in table 6, preferably independently under control of a PglpF promoter, and
   iii. optionally, a nucleic acid sequence encoding vag, preferably under control of a PglpF or Plac promoter, and
b) culturing said genetically modified cell in a carbon-source containing culture medium in the presence of LNT to be internalized into said genetically modified cell, and
c) to produce said human milk oligosaccharide (HMO) having at least four monosaccharide units, in particular LNFP-I, LNFP-II, LNFP-V, LNDFH-1 or LNFDH-II, by said genetically modified cell, and
d) retrieving the human milk oligosaccharide (HMO) having at least four monosaccharide units, in particular LNFP-I, LNFP-II, LNFP-V, LNDFH-1 or LNFDH-II, from the culture medium and/or the genetically modified cell.

In particular, the present invention relates to a method for producing LST-a, LST-b or DSLST, said method comprising:
a) obtaining a genetically modified cell comprising
   i. a nucleic acid sequence encoding a transporter protein capable of importing LNT, selected from the group of ABV importers or MFS importers in Table 5, in particular an importer selected from the group consisting of TP ID NO: 1, 2, 3, 8, 9, and 10, and
   ii. at least one nucleic acid sequence encoding a sialyltransferase, such as a heterologous alpha-2,3-sialyltransferase and/or alpha-2,6-sialyltransferase, or as listed in table 6, preferably independently under control of a PglpF promoter, and
   iii. optionally, a nucleic acid sequence encoding vag, preferably under control of a PglpF or Plac promoter, and
b) culturing said genetically modified cell in a carbon-source containing culture medium in the presence LNT to be internalized into said genetically modified cell, and
c) to produce said human milk oligosaccharide (HMO) having at least four monosaccharide units, in particular LST-a or LST-b, by said genetically modified cell, and
d) retrieving the human milk oligosaccharide (HMO) having at least four monosaccharide units, in particular LST-a or LST-b, from the culture medium and/or the genetically modified cell.

In particular, the present invention relates to a method for producing LNH, said method comprising:
a) obtaining a genetically modified cell comprising
   i. a nucleic acid sequence encoding a transporter protein capable of importing LNT, selected from the group of ABV importers or MFS importers in Table 5, in particular an importer selected from the group consisting of TP ID NO: 1, 2, 3, 4, 8, 9, and 10, and
   ii. a nucleic acid sequences encoding a β-1,6-N-acetylglucosaminyl-transferase, such as Csp2 or as listed in table 6, preferably independently under control of a PglpF promoter, and
   iii. optionally, a nucleic acid sequence encoding vag, preferably under control of a PglpF or Plac promoter, and
b) culturing said genetically modified cell in a carbon-source containing culture medium in the presence LNT to be internalized into said genetically modified cell, and
c) to produce said human milk oligosaccharide (HMO) having at least four monosaccharide units, in particular LNH, by said genetically modified cell, and
d) retrieving the human milk oligosaccharide (HMO) having at least four monosaccharide units, in particular LNH, from the culture medium and/or the genetically modified cell.

In particular, the present invention relates to a method for producing LNFP-III, LNFP-VI, or LNDFH-III, said method comprising:
a) obtaining a genetically modified cell comprising
   i. a nucleic acid sequence encoding a transporter protein capable of importing LNnT, selected from the group of ABV importers or MFS importers in Table 8, in particular an importer selected from the group consisting of TP ID NO: 1, 2, 3, 4, 8, and 11, and
   ii. at least one nucleic acid sequence encoding an alpha-1,3-fucosylsyltransferase as listed in table 9, preferably under control of a PglpF promoter, and
   iii. optionally, a nucleic acid sequence encoding vag, preferably under control of a PglpF or Plac promoter, and
b) culturing said genetically modified cell in a carbon-source containing culture medium in the presence of LNnT to be internalized into said genetically modified cell, and
c) to produce said human milk oligosaccharide (HMO) having at least four monosaccharide units, in particular LNFP-III, LNFP-VI, or LNDFH-III, by said genetically modified cell, and
d) retrieving the human milk oligosaccharide (HMO) having at least four monosaccharide units, in particular LNFP-III, LNFP-VI, or LNDFH-III, from the culture medium and/or the genetically modified cell.

In particular, the present invention relates to a method for producing LNFP-III, LNFP-VI, or LNDFH-III, said method:
a) obtaining a genetically modified cell comprising
   i. a nucleic acid sequence encoding a transporter protein capable of importing LNnT, selected from the group of ABV importers or MFS importers in Table 8, in particular an importer selected from the group consisting of TP ID NO: 1, 2, 3, 4, 8, and 11, and
   ii. at least one nucleic acid sequence encoding FucT, FutA or CafC, preferably under control of a PglpF promoter, and
   iii. optionally, a nucleic acid sequence encoding vag, preferably under control of a PglpF or Plac promoter, and
b) culturing said genetically modified cell in a carbon-source containing culture medium in the presence of LNnT to be internalized into said genetically modified cell, and
c) to produce said human milk oligosaccharide (HMO) having at least four monosaccharide units, in particular LNFP-III, LNFP-VI, or LNDFH-III, by said genetically modified cell, and
d) retrieving the human milk oligosaccharide (HMO) having at least four monosaccharide units, in particular LNFP-III, LNFP-VI, or LNDFH-III, from the culture medium and/or the genetically modified cell.

In particular, the present invention relates to a method for producing LST-c, said method comprising:
a) obtaining a genetically modified cell comprising
   i. a nucleic acid sequence encoding a transporter protein capable of importing LNnT, selected from the group of ABV importers or MFS importers in Table 8, in particular an importer selected from the group consisting of TP ID NO: 1, 2, 3, 4, 8, and 11, and
   ii. at least one nucleic acid sequence encoding an alpha-2,6-sialyltransferase, such as HAC1268, preferably under control of a PglpF promoter, and
   iii. optionally, a nucleic acid sequence encoding vag, preferably under control of a PglpF or Plac promoter, and
b) culturing said genetically modified cell in a carbon-source containing culture medium in the presence of LNnT to be internalized into said genetically modified cell, and
c) to produce said human milk oligosaccharide (HMO) having at least four monosaccharide units, in particular LST-c, by said genetically modified cell, and
d) retrieving the human milk oligosaccharide (HMO) having at least four monosaccharide units, in particular LST-c, from the culture medium and/or the genetically modified cell.

In particular, the present invention relates to a method for producing LNnH, said method comprising:
a) obtaining a genetically modified cell comprising
   i. a nucleic acid sequence encoding a transporter protein capable of importing LNnT, selected from the group of ABV importers or MFS importers in Table 8, in particular an importer selected from the group consisting of TP ID NO: 1, 2, 3, 4, 8, and 11, and
   ii. a nucleic acid sequences encoding a β-1,6-N-acetylglucosaminyl-transferase, such as Csp2, and
   iii. optionally, a nucleic acid sequence encoding vag, preferably under control of a PglpF or Plac promoter, and
b) culturing said genetically modified cell in a carbon-source containing culture medium in the presence of LNnT to be internalized into said genetically modified cell, and
c) to produce said human milk oligosaccharide (HMO) having at least four monosaccharide units, in particular LNnH, by said genetically modified cell, and
d) retrieving the human milk oligosaccharide (HMO) having at least four monosaccharide units, in particular LNnH, from the culture medium and/or the genetically modified cell.

In particular, the present invention relates to a method for producing pLNnH or pLNH-I, said method comprising:
a) obtaining a genetically modified cell comprising
   i.a nucleic acid sequence encoding a transporter protein capable of importing LNnT, selected from the group of ABV importers or MFS importers in Table 8, in particular, an importer selected from the group consisting of TP ID NO: 1, 2, 3, 4, 8, and 11, and
   ii. Two nucleic acid sequences one encoding a β-1,3-N-acetyl-glucosaminyl-transferase, such as LgtA and the second one encoding β-1,4-galactosyl transferase, such as GalT or β-1,3-galactosyl transferase, such as GalTK, preferably independently under control of a PglpF promoter, and
   iii. optionally, a nucleic acid sequence encoding vag, preferably under control of a PglpF or Plac promoter, and
b) culturing said genetically modified cell in a carbon-source containing culture medium in the presence of LNnT to be internalized into said genetically modified cell, and
c) to produce said human milk oligosaccharide (HMO) having at least four monosaccharide units by, in particular pLNnH or pLNH-1, said genetically modified cell, and
d) retrieving the human milk oligosaccharide (HMO) having at least four monosaccharide units, in particular pLNnH or pLNH-1, from the culture medium and/or the genetically modified cell.

In particular, the present invention relates to a method for producing DFL, said method comprising culturing a genetically modified cell comprising:
a) obtaining said genetically modified cell comprising
   i. a nucleic acid sequence encoding a transporter protein capable of importing 2′FL or 3FL, selected from the group of ABV importers or MFS importers in Table 11, in particular an importer selected from the group consisting of TP ID NO: 2, 3, 4, 13, 14, 17 and 18, and
   ii. At least nucleic acid sequences one encoding a heterologous alpha-1,2-fucosylsyltransferase or an alpha-1,3-fucosylsyltransferase in table 12, such as FutC, wbgL, FutA or FucT, preferably independently under control of a PglpF promoter, and
   iii. optionally, a nucleic acid sequence encoding vag, preferably under control of a PglpF or Plac promoter, and
b) culturing said genetically modified cell in a carbon-source containing culture medium in the presence of 2′FL or 3FL to be internalized into said genetically modified cell, and
c) to produce said human milk oligosaccharide (HMO) having at least four monosaccharide units, in particular DFL, by said genetically modified cell, and
d) retrieving the human milk oligosaccharide (HMO) having at least four monosaccharide units, in particular DFL, from the culture medium and/or the genetically modified cell.

In particular, the present invention relates to a method for producing FSL, said method comprising culturing a genetically modified cell comprising:
a) obtaining said genetically modified cell comprising
   i.a nucleic acid sequence encoding a transporter protein capable of importing 3FL, selected from the group of ABV importers or MFS importers in Table 11, in particular an importer selected from the group consisting of TP ID NO: 2, 3, 4, 13, 14, 17 and 18, and
   ii. At least nucleic acid sequences one encoding a alpha-2,3-sialyltransferase, such as Ccol, preferably independently under control of a PglpF promoter, and
   iii. optionally, a nucleic acid sequence encoding vag, preferably under control of a PglpF or Plac promoter, and
b) culturing said genetically modified cell in a carbon-source containing culture medium in the presence of 3FL to be internalized into said genetically modified cell, and
c) to produce said human milk oligosaccharide (HMO) having at least four monosaccharide units, in particular SFL, by said genetically modified cell, and
d) retrieving the human milk oligosaccharide (HMO) having at least four monosaccharide units, in particular SFL, from the culture medium and/or the genetically modified cell.

In particular, the present invention relates to a method for producing LNDFH-1, said method comprising:
a) obtaining a genetically modified cell comprising
   i. a nucleic acid sequence encoding a transporter protein capable of importing LNFP-I, selected from the group of ABV importers or MFS importers in Table 11, in particular, an importer selected from the group consisting of TP ID NO: 2, 3, 4, 13, 14, 17 and 18, and
   ii. At least nucleic acid sequences one encoding a heterologous alpha-1,3/4-fucosylsyltransferase, such as FucTIII, preferably under control of a PglpF promoter, and
   iii. optionally, a nucleic acid sequence encoding vag, preferably under control of a PglpF or Plac promoter, and
b) culturing said genetically modified cell in a carbon-source containing culture medium in the presence of LNDFH-1 to be internalized into said genetically modified cell, and
c) to produce said human milk oligosaccharide (HMO) having at least four monosaccharide units, in particular LNDFH-1, by said genetically modified cell, and
d) retrieving the human milk oligosaccharide (HMO) having at least four monosaccharide units in particular LNDFH-1, from the culture medium and/or the genetically modified cell.

In particular, the present invention relates to a method for producing LNDFH-1, said method comprising:
a) obtaining a genetically modified cell comprising
   i. a nucleic acid sequence encoding a transporter protein capable of importing LNFP-I, selected from the group of ABV importers or MFS importers in Table 11, in particular, an importer selected from the group consisting of TP ID NO: 2, 3, 4, 13, 14, 17 and 18, and
   ii. At least nucleic acid sequences one encoding FucTIII, preferably under control of a PglpF promoter, and
   iii. optionally, a nucleic acid sequence encoding vag, preferably under control of a PglpF or Plac promoter, and
b) culturing said genetically modified cell in a carbon-source containing culture medium in the presence of LNDFH-1 to be internalized into said genetically modified cell, and
c) to produce said human milk oligosaccharide (HMO) having at least four monosaccharide units, in particular LNDFH-1, by said genetically modified cell, and
d) retrieving the human milk oligosaccharide (HMO) having at least four monosaccharide units, in particular LNDFH-1, from the culture medium and/or the genetically modified cell.

### Retrieving/Harvesting

The human milk oligosaccharide (HMO) having at least four monosaccharide units is retrieved from the culture medium and/or the genetically modified cell. In the present context, the term “retrieving” is used interchangeably with the term “harvesting”.

Both “retrieving” and “harvesting” in the context relates to collecting the produced HMO(s) following the termination of fermentation. In one or more exemplary embodiments it may include collecting the HMO(s) included in both the biomass (i.e., the host cells) and cultivation media, i.e., before/without separation of the fermentation broth from the biomass. In other embodiments, the produced HMOs may be collected separately from the biomass and fermentation broth, i.e., after/following the separation of biomass from cultivation media (i.e., fermentation broth).

The separation of cells from the medium can be carried out with any of the methods well known to the skilled person in the art, such as any suitable type of centrifugation or filtration. The separation of cells from the medium can follow immediately after harvesting the fermentation broth or be carried out at a later stage after storing the fermentation broth at appropriate conditions. Recovery of the produced HMO(s) from the remaining biomass (or total fermentation) include extraction thereof from the biomass (i.e., the production cells).

After recovery from fermentation, HMO(s) are available for further processing and purification.

### Manufacturing of HMOs

The present invention further relates to a method for producing a human milk oligosaccharide (HMO) having at least four monosaccharide units, said method comprising culturing a genetically modified cell according to the present invention in a culture medium with a suitable carbon-source and said acceptor oligosaccharide of at least three monosaccharide units; and producing said human milk oligosaccharide (HMO) having at least four monosaccharide units by said genetically modified cell, and retrieving the human milk oligosaccharide (HMO) having at least four monosaccharide units from the culture medium and/or the genetically modified cell.

To produce one or more HMOs, the genetically engineered cells as described herein are cultivated according to the procedures known in the art in the presence of a suitable carbon and energy source, e.g. glucose, sucrose, fructose, xylose and glycerol, and a suitable acceptor, i.e., an acceptor oligosaccharide of at least 3, such as at least 4, monosaccharide units, and the produced HMO blend is harvested from the cultivation media and the microbial biomass formed during the cultivation process. Thereafter, the HMOs are purified according to the procedures known in the art, e.g., such as described in WO2015188834, WO2017182965 or WO2017152918, and the purified HMOs are used as nutraceuticals, pharmaceuticals, or for any other purpose, e.g., for research.

Preferably, the culturing or fermentation (used interchangeably herein) comprises (a) a first phase of exponential cell growth in a culture medium ensured by a carbon-source, and (b) a second phase of cell growth in a culture medium run under carbon limitation, where the carbon-source is added continuously. By carbon (sugar) limitation is meant the stage in the fermentation where the growth rate is kinetically controlled by the concentration of the carbon source (sugar) in the culture broth, which in turn is determined by the rate of carbon addition (sugar feed-rate) to the fermenter.

At the end of culturing, the oligosaccharide as product can be accumulated both in the intra- and the extracellular matrix.

After carrying out the method of this invention, the HMO of at least four or five or six monosaccharide units formed can be collected from the cell culture or fermentation broth in a conventional manner.

The method according to the present invention comprises cultivating the genetically engineered microbial cell in a culture medium which is designed to support the growth of microorganisms, and which contains one or more carbohydrate sources or just carbon-source, such as selected from the group consisting of glucose, sucrose, fructose, xylose and glycerol. In one or more exemplary embodiments, the culturing media is supplemented with one or more energy and carbon sources selected form the group containing glycerol, sucrose and glucose.

Manufacturing of HMOs is typically accomplished by performing cultivation in larger volumes. The term “manufacturing” and “manufacturing scale” in the meaning of the invention defines a fermentation with a minimum volume of 100 L, such as 1000 L, such as 10.000 L, such as 100.000 L, such as 200.000 L culture broth. Usually, a “manufacturing scale” process is defined by being capable of processing large volumes of a preparation containing the product of interest and yielding amounts of the complex HMO product of interest that meet, e.g., in the case of a therapeutic compound or composition, the demands for toxicity tests, clinical trials as well as for market supply. In addition to the large volume, a manufacturing scale method, as opposed to simple lab scale methods like shake flask cultivation, is characterized by the use of the technical system of a bioreactor (fermenter) which is equipped with devices for agitation, aeration, nutrient feeding, monitoring and control of process parameters (pH, temperature, dissolved oxygen tension, back pressure, etc.). To a large extent, the behavior of an expression system in a lab scale method, such as shake flasks, benchtop bioreactors or the deep well format described in the examples of the disclosure, does allow to predict the behavior of that system in the complex environment of a bioreactor.

With regards to the suitable cell medium used in the fermentation process, there are no limitations. The culture medium may be semi-defined, i.e., containing complex media compounds (e.g., yeast extract, soy peptone, casamino acids, etc.), or it may be chemically defined, without any complex compounds. Where sucrose is used as the carbon and energy source, a minimal medium might be preferable.

In one embodiment, the culture medium does not contain lactose.

In one or more exemplary embodiments, the culturing media contains sucrose as the sole carbon and energy source. In one or more exemplary embodiments, the genetically engineered cell comprises one or more heterologous nucleic acid sequence encoding one or more heterologous polypeptide(s) which enables utilization of sucrose as sole carbon and energy source of said genetically engineered cell.

In one or more exemplary embodiments, the genetically engineered cell comprises a PTS-dependent sucrose utilization system, further comprising the scrYA and scrBR operons as described in WO2015197082 (hereby incorporated by reference).

### Manufactured Product

The term “manufactured product” according to the use of the genetically engineered cell or the nucleic acid construct refer to the one or more HMOs intended as the one or more product HMO(s). The various products are described above.

Advantageously, the methods disclosed herein provides both a decreased ratio of by-product to product and an increased overall yield of the product (and/or HMOs in total). This, less by-product formation in relation to product formation facilitates an elevated product production and increases efficiency of both the production and product recovery process, providing superior manufacturing procedure of HMOs.

The manufactured product may be a powder, a composition, a suspension, or a gel comprising one or more HMOs.

Various embodiments of present disclosure are described in the following clauses
1. A genetically modified cell comprising a recombinant nucleic acid sequence and/or a cluster of recombinant nucleic acid sequences encoding a transporter protein and/or a cluster of proteins capable of importing an acceptor oligosaccharide of at least three monosaccharide units into said cell, wherein said transporter protein and/or cluster of proteins is selected from the group consisting of mutated lactose permease, as shown in table 2, and ABC-importers or MFS importers from a gram-positive bacterium, as shown in table 1.
2. The genetically modified cell according to item 1, comprising a recombinant nucleic acid sequence and/or a cluster of recombinant nucleic acid sequences encoding a transporter protein and/or a cluster of proteins capable of importing an acceptor oligosaccharide of at least four monosaccharide units into said cell, wherein the transporter is selected from ABC-importers or MFS importers from a gram-positive bacterium, as shown in table 1.
3. The genetically modified cell according to item 1 or 2, wherein the cluster of transporter proteins capable of importing an acceptor oligosaccharide of at least three monosaccharide units into said cell is an ABC transporter from a gram-positive cell.
4. The genetically modified cell according to item 3, wherein the ABC transporter is selected from the group consisting of TP ID: 5, 6, 7, 8, 9, 10, 11, 12, 15, 16, 17 and 18 or a subset of ABC transporters selected from the group consisting of TP ID: 8, 9, 10, 11, 17 and 18.
5. The genetically modified cell according to item 1 to 3, wherein the cluster of transporter proteins capable of importing an acceptor oligosaccharide of at least three monosaccharide units into said cell is an ABC importer selected from the group consisting of:
   a. Blon2177, 2176 and 2175 (TP ID: 8 in table 1) comprising three sub-units with the amino acid sequences comprising or consisting of SEQ ID NO: 13, 14 and 15 or amino acid sequences with at least 80%, such as 85%, such as 90%, such as 95% identity to SEQ ID: 13, 14 and 15;
   b. RHOM_04095, 04100, 04105 (TP ID: 9 in table 1) comprising three sub-units with the amino acid sequences comprising or consisting of SEQ ID NO: 35, 36 and 37 or amino acid sequences with at least 80%, such as 85%, such as 90%, such as 95% identity to SEQ ID: 35, 36 and 37;
   c. BBPC_1775, 1776, 1777 (TP ID:18 in table 1) comprising three sub-units with the amino acid sequences comprising or consisting of SEQ ID NO: 47, 48 and 49 or amino acid sequences with at least 80%, such as 85%, such as 90%, such as 95% identity to SEQ ID: 47, 48 and 49;
   d. Bbr_0527, 0528, 0530, 0531 (TP ID: 11 in table 1). comprising four sub-units with the amino acid sequences comprising or consisting of SEQ ID NO: 28, 29, 30 and 50 or amino acid sequences with at least 80%, such as 85%, such as 90%, such as 95% identity to SEQ ID: 28, 29, 30 and 50;
   e. HMPREF0373_02960, 0373_02961, 0373_02962 (TP ID: 10 in table 1) comprising three sub-units with the amino acid sequences comprising or consisting of SEQ ID NO: 38, 39 and 40 or amino acid sequences with at least 80%, such as 85%, such as 90%, such as 95% identity to SEQ ID: 38, 39 and 40; and
   f. BBKW_1838, 1839, 1840 (TP ID: 17 in table 1) comprising three sub-units with the amino acid sequences comprising or consisting of SEQ ID NO: 44, 45 and 46 or amino acid sequences with at least 80%, such as 85%, such as 90%, such as 95% identity to SEQ ID: 44, 45 and 46 or with the amino acid sequences comprising or consisting of SEQ ID NO: 41, 42 and 43 or amino acid sequences with at least 80%, such as 85%, such as 90%, such as 95% identity to SEQ ID: 41, 42 and 43.
6. The genetically modified cell according to item 1 or 2, wherein transporter protein capable of importing an acceptor oligosaccharide of at least three monosaccharide units is an MFS transporter from a gram-positive cell.
7. The genetically modified cell according to item 6, wherein the MFS transporter is selected from the group consisting of TP ID: 1, 2, 3, 4, 13 and 14.
8. The genetically modified cell according to item 1 or 2 or 6 or 7, wherein the transporter proteins capable of importing an acceptor oligosaccharide of at least three monosaccharide units into said cell is an MFS transporter selected from the group consisting of:
   a. Blon:0247 (TP ID: 1 in table 1) comprising or consisting of an amino acid sequence of SEQ ID NO: 2 or an amino acid sequence with at least 80%, such as 85%, such as 90%, such as 95% identity to SEQ ID: 2;
   b. Blon_0431 (TP ID: 2 in table 1 comprising or consisting of an amino acid sequence of SEQ ID NO: 6 or an amino acid sequence with at least 80%, such as 85%, such as 90%, such as 95% identity to SEQ ID: 6;
   c. Blon_0788 (TP ID: 3 in table 1) comprising or consisting of an amino acid sequence of SEQ ID NO: 7 or an amino acid sequence with at least 80%, such as 85%, such as 90%, such as 95% identity to SEQ ID: 7;
   d. Blon_0962 (TP ID: 13 in table 1) comprising or consisting of an amino acid sequence of SEQ ID NO: 12 or an amino acid sequence with at least 80%, such as 85%, such as 90%, such as 95% identity to SEQ ID: 12;
   e. Blon_2307 (TP ID: 14 in table 1) comprising or consisting of an amino acid sequence of SEQ ID NO: 19 or an amino acid sequence with at least 80%, such as 85%, such as 90%, such as 95% identity to SEQ ID: 19; and
   f. Blon_2400 (TP ID: 4 in table 1) comprising or consisting of an amino acid sequence of SEQ ID NO: 27 or an amino acid sequence with at least 80%, such as 85%, such as 90%, such as 95% identity to SEQ ID: 27.
9. The genetically modified cell according to item 1, wherein transporter protein capable of importing an acceptor oligosaccharide of at least three monosaccharide units is a mutated lactose permease (LacY) as shown in table 2.
10. The genetically modified cell according to item Error! Reference source not found. or 9, wherein the mutated lactose permease is selected from a lactose permease of SEQ ID NO: 1 or a lactose permease with 90% identity to SEQ ID NO: 1, wherein the lactose permease has one or more mutations selected from the group consisting of Y236N, Y236H, S306T, A177V, H322N, 1303F, Y236H+S306T, 177V+Y236H, A177V+1303F, A177V+H322N, A177V+S306T or A177V+Y236N+S306T and wherein the mutation is at the corresponding position in SEQ ID NO: 1.
11. The genetically modified cell according to any one of items 1 to 10, wherein the cell further comprises at least one recombinant nucleic acid sequence encoding at least one glycosyltransferase capable of transferring a glycosyl residue from a glycosyl donor to said acceptor oligosaccharide to synthesize an oligosaccharide product having at least four monosaccharide units.
12. The genetically modified cell according to item 11, wherein the glycosyltransferase is selected from the group consisting of fucosyltransferases, galactosyltransferases, glucosaminyltransferases, sialic acid transferases, N-acetylglucosaminyl transferases and N-acetylglucosaminyl transferases.
13. The genetically modified cell according to item 11 or 12, wherein genetically modified cell contains one and/or two recombinant nucleic acid sequence encoding one and/or two glycosyltransferases.
14. The genetically modified cell according any one of items 1 to 13, wherein the cell comprise one or more pathways to produce nucleotide-activated sugar selected from the group consisting of glucose-UDP-GlcNac, GDP-fucose, UDP-galactose, UDP-glucose, UDP-N-acetylglucosamine, UDP-N-acetylgalactosamine and CMP-N-acetylneuraminic acid.
15. The genetically modified cell according to any one of the preceding items, wherein the cell further comprises a nucleic acid sequence encoding a MFS transporter protein capable of exporting the human milk oligosaccharide product having at least four monosaccharide units into the extracellular medium.
16. The genetically modified cell according to any one of the preceding items, wherein the MFS transporter protein capable of exporting the human milk oligosaccharide product having at least four monosaccharide units is Vag.
17. The genetically modified cell according to any one of the preceding items, wherein the genetically modified cell does not express a functional lactose importer.
18. The genetically modified cell according to item 17, wherein the genetically modified cell is lacY negative.
19. The genetically modified cell according to any one of the preceding items, wherein said modified cell is a cell.
20. The genetically modified cell according to any one of the preceding items, wherein said modified cell is a bacterium or a fungus.
21. The genetically modified cell according to item 20, wherein said modified cell is selected from the group consisting of Escherichia coli, Corynebacterium glutamicum, Lactococcus lactis, Bacillus subtilis, Streptomyces lividans, Pichia pastoris and Saccharomyces cerevisiae.
22. The genetically modified cell according to item 20, wherein said fungi is selected from a yeast cell of the genera Komagataella, Kluyveromyces, Yarrowia, Pichia, Saccaromyces, Schizosaccharomyces or Hansenula or from a filamentous fungous of the genera Aspargillus, Fusarium or Thricoderma.
23. The genetically modified cell according to item 20, wherein said bacterium is a gram-negative bacterium selected from the group consisting of Escherichia sp., and Campylobacter sp.
24. A method for producing an oligosaccharide having at least four monosaccharide units, said method comprising culturing a genetically modified cell comprising:
i. a recombinant nucleic acid sequence and/or a cluster of recombinant nucleic acid sequences encoding a transporter protein and/or a cluster of proteins capable of importing an acceptor oligosaccharide of at least three monosaccharide units into said cell, and
   ii. at least one recombinant nucleic acid encoding at least one glycosyltransferase capable of transferring a glycosyl residue from a glycosyl donor to said acceptor oligosaccharide to synthesize a human milk oligosaccharide product having at least four monosaccharide units,
wherein the recombinant nucleic acid sequence and/or the cluster of recombinant nucleic acid sequences encoding a transporter protein and/or a cluster of proteins capable of importing said acceptor oligosaccharide of at least three monosaccharide units is selected from the group consisting of mutated lactose permease from table 2 and ABC-importers or MFS importers from a gram-positive bacterium, as shown in table 1.
25. The method according to item 24, wherein the recombinant nucleic acid sequence and/or a cluster of recombinant nucleic acid sequences encoding a transporter protein and/or a cluster of proteins capable of importing an acceptor oligosaccharide of at least three monosaccharide units is selected from the groups of ABC or MFS importers of item Error! Reference source not found. or Error! Reference source not found.
26. The method according to any one of items 24 or 25, wherein the glycosyltransferase is selected from the group consisting of fucosyltransferases, galactosyltransferases, glucosaminyltransferases, sialic acid transferases, N-acetylglucosaminyl transferases and N-acetylglucosaminyl transferases.
27. The method according to any one of items 24 to 26, wherein the genetically modified cell contains one and/or two recombinant nucleic acid sequences encoding one and/or two glycosyltransferases.
28. The method according to any one of items 24 to 27, wherein the genetically modified cell further comprises a nucleic acid sequence encoding a MFS transporter protein capable of exporting the human milk oligosaccharide product having at least four monosaccharide units into the extracellular medium.
29. The method according to any of items 24 to 28, wherein the acceptor oligosaccharide having at least three or four or five monosaccharide units is a neutral oligosaccharide.
30. The method according to item 29, wherein the neutral oligosaccharide does not contain a fucosyl unit.
31. The method according to any of items 24 to 30, wherein the acceptor oligosaccharide having at least three monosaccharide units is LNTII and the acceptor oligosaccharide having at least four monosaccharide units is LNT or LNnT.
32. The method according to any of items 24 to 29, wherein the acceptor oligosaccharide having at least three monosaccharide units is 2′FL or 3FL and the acceptor oligosaccharide having at least five monosaccharide units is LNFP-I.
33. The method according to any of items 24 to 28, wherein the human milk oligosaccharide (HMO) having at least four monosaccharide units is an HMO of only four monosaccharide units.
34. The method according to any of items 24 to 33, wherein the human milk oligosaccharide (HMO) produced by the cell having at least four monosaccharide units is LNT or LNnT or DFL or SFL.
35. The method according to anyone of items 24 to 33, wherein the human milk oligosaccharide (HMO) produced by the cell is an HMO of at least five monosaccharide units.
36. The method according to anyone of items 24 to 32, wherein the human milk oligosaccharide (HMO) produced by the cell has five monosaccharide units and is selected from the list consisting of LNFP-I, LNFP-II, LNFP-III, LNFP-V, LNFP-VI, LST-a, LST-b and LST-c.
37. The method according to anyone of items 24 to 32, wherein the human milk oligosaccharide (HMO) produced by the cell has six monosaccharide units and is selected from the list consisting of LNH, LNnH, pLNnH, pLNH-I, DSLNT, LNDFH-I, LNDFH-II and LNDFH-III.
38. A method according to any one of items 24 to 37, wherein the glycosyl doner is a nucleotide-activated sugar selected from the group consisting of glucose-UDP-GlcNac, GDP-fucose, UDP-galactose, UDP-glucose, UDP-N-acetylglucosamine, UDP-N-acetylgalactosamine and CMP-N-acetylneuraminic acid.
39. The method according to item 38, wherein the glycosyl donor is synthesized by the genetically engineered cells and/or is exogenously added to the culture medium.
40. The method according to any one of items 24 to 39, wherein the acceptor oligosaccharide of at least three monosaccharide units is exogenously added to the culture medium.
41. The method according to any one of items 24 to 39, wherein the acceptor oligosaccharide of at least three monosaccharide units has been produced by microbial fermentation.
42. The method according to any one of items 24 to 41, wherein the genetically modified cell lacks enzymatic activity liable to degrade the oligosaccharide of at least three monosaccharide units.
43. The method according to any one of items 24 to 42, wherein the method comprises cultivating the genetically engineered cell in a culture medium which contains one or more carbohydrate sources.
44. The method according to item 43, wherein the culture medium comprises a carbohydrate source selected from the group consisting of glucose, sucrose, fructose, xylose and glycerol.
45. The method according to any one of items 24 to 44, wherein the culture medium in which the cultivation is conducted does not contain lactose.
46. The method according to any one of items 24 to 45, wherein the human milk oligosaccharide (HMO) having at least four monosaccharide units is retrieved from the culture medium and/or the genetically modified cell.
47. The method according to any one of items 24 to 46, wherein the genetically modified cell is a fungus or a prokaryotic cell.
48. The method according to item 47, wherein said fungus is selected from a yeast cells of the genera Komagataella, Kluyveromyces, Yarrowia, Pichia, Saccaromyces, Schizosaccharomyces or Hansenula or from a filamentous fungi of the genera Aspargillus, Fusarium or Thricoderma.
49. The method according to item 47, wherein the genetically modified cell is a gram-negative bacterium, such as Escherichia sp. or Campylobacter sp.

### EXAMPLES

### Methods

Unless stated otherwise, standard techniques, vectors, control sequence elements, and other expression system elements known in the field of molecular biology are used for nucleic acid manipulation, transformation, and expression. Such standard techniques, vectors, and elements can be found, e.g., in: Ausubel et al. (eds.), Current Protocols in Molecular Biology (1995) (John Wiley & Sons); Sambrook, Fritsch, & Maniatis (eds.), Molecular Cloning (1989) (Cold Spring Harbor Laboratory Press, NY); Berger & Kimmel, Methods in Enzymology 152: Guide to Molecular Cloning Techniques (1987) (Academic Press); Bukhari et al. (eds.), DNA Insertion Elements, Plasmids and Episomes (1977) (Cold Spring Harbor Laboratory Press, NY); Miller, J. H. Experiments in molecular genetics (1972.) (Cold spring Harbor Laboratory Press, NY)

The embodiments described below are selected to illustrate the invention and are not limiting the invention in any way.

### Transporter (Importer) Proteins:

As the outline of the present invention, several importer proteins were collected following an in-silico approach, where information sources such as scientific articles or databases, e.g., the KEGG and CAZY databases, were exploited to identify transporter proteins that are potentially capable of importing oligosaccharides of 3 units or more, such as LNT-II, LNT, LNnT, LNFP-1 etc.

The group of collected sequences is represented by many different types of transporter proteins, including proteins of the Major Facilitator Superfamily (MFS), ATP-binding cassette transporters (ABC transporters) and porter proteins (e.g., mutant variants of the E. coli LacY symporter) (see Table 1 and 2 respectively).

Examples 1 to 4 below illustrates how the identified transporters can be tested for their ability to import the desired acceptor oligosaccharide.

### Example 1: Import of LNT-11 by E. coli DH1 K12 Cells to Produce LNnT or LNT

The present example sets out to test the ability of lactose permease mutants to import LNT into a host cell for further decoration to produce LNT or LNnT.

### Strain Design

In the present example a modified E. coli DH1 K12 strain is engineered to produce the tetrasaccharides lacto-N-neotetraose (LNnT) or lacto-N-tetraose (LNT) by enabling it to take up LNT-II from the culture medium. The strain is engineered to expresses a lactose permease (LacY) mutant with increased LNT-II affinity from Table 2 and a β-1,4- or a β-1,3-galactosyltransferase, respectively from Table 3. Some of the LacY mutants have previously been described as importers of maltotriose by Olsen 1993 et al. J Bacteriol. 175(19):6269-75 but has not been associated with LNT-II import.

TABLE 2
List of exemplary mutants of the E. coli DH1 K12 lactose permease
LacY (SEQ ID NO: 1) that could be useful for the import of LNT-II

| Variant | | | Wild-type | Mutant |
|---|---|---|---|---|
| Name | Mutation | Position | amino acid | amino acid |
| mut1 | Y236N | 236 | Tyr | Asn |
| mut2 | Y236H | 236 | Tyr | His |
| mut3 | S306T | 306 | Ser | Thr |
| mut4 | A177V | 177 | Ala | Val |
| mut5 | H322N | 322 | His | Asn |
| Mut6 | I303F | 303 | Ile | Phe |
| mut6 | Y236H, | 236 | Tyr | His |
| | S306T | 306 | Ser | Thr |
| mut7 | A177V, | 177 | Ala | Val |
| | Y236H | 236 | Tyr | His |
| mut8 | A177V, | 177 | Ala | Val |
| | I303F | 303 | Ile | Phe |
| mut9 | A177V, | 177 | Ala | Val |
| | H322N | 322 | His | Asn |
| mut10 | A177V, | 177 | Ala | Val |
| | S306T | 306 | Ser | Thr |
| mut11 | A177V, | 177 | Ala | Val |
| | Y236N, | 236 | Tyr | Asn |
| | S306T | 306 | Ser | Thr |

It is understood that the first letter in the mutation listed in Table 2 corresponds to the amino acid at the indicated position of SEQ ID NO: 1 and that the second letter in the mutation is the amino acid that substitutes the original amino acid. If multiple mutations are listed in connection with a variant name, it is understood that the variant contains all the mutations listed.

TABLE 3
List of glycosyltransferases that can be expressed by a LNT or LNnT HMO-producing
strain that also expresses a LNT-II importer of bacterial origin (strain X)

| Protein | | | | HMO |
|---|---|---|---|---|
| Name | Enzyme Type | GenBank ID | Origin | product |
| GalT | β-1,4-galactosyl | WP_001262061.1 | Helicobacter pylori | LNnT |
| | transferase | | | |
| LgtB | β-1,4-galactosyl | AAF42257.1 | Neisseria meningitidis | LNnT |
| | transferase | | MC58 | |
| WbgO | β-1,3-galactosyl | WP_000582563.1 | E. coli 055:H7 | LNT |
| | transferase | | | |
| cpslBJ | β-1,3-galactosyl | AB050723.1 | Streptococcus | LNnT |
| | transferase | | agalactiae type Ib OI2 | |
| Jhp0563 | β-1,3-galactosyl | AEZ55696.1 | Helicobacter pylori | LNnT |
| | transferase | | | |
| GalTK | β-1,3-galactosyl | WP_111735921.1 | Helicobacter pylori | LNT |
| | transferase | | ATCC 43504 | |
| CvB3GalT | β-1,3-galactosyl | WP_080969100.1 | Chromobacterium | LNT |
| | transferase | | violaceum | |

### Strain Construction

During strain construction, the genome of the platform strain MOO that is well-suited for HMO production (WO2019/123324, see also table 4) can be engineered using methods that are well-known in the art (e.g., gene gorging by Herring & Blattner 2004 Conditional lethal amber mutations in essential Escherichia coli genes. J Bacteriol 186:2673-2681), so that two novel expression cassettes are integrated at non-essential genomic loci: a) an expression cassette containing a constitutive or inducible promoter (e.g., Plac, PglpF, Ptac, Pbad, PosmY) controlling the expression of a mutated coding sequence of the lacY gene (Table 2) and a terminator sequence (e.g., T1) and b) an expression cassette containing a constitutive or inducible promoter (e.g., Plac, PglpF, Ptac, Pbad, PosmY) controlling the expression of the coding sequence of a gene encoding a given galactosyltransferase (Table 3) and a terminator sequence (e.g., T1). The resulting strain is hereby denoted as strain X (Table 4).

Exemplary genotypes for this example are provided in Table 4 below.

TABLE 4
Exemplary genotypes of strains described in the present example

| Strain ID | Genotype |
|---|---|
| DH1 | F−λ− endA1 recA1 relA1 gyrA96 thi-1 |
| | glnV44 hsdR17(rK−mK−) |
| MDO | E coli DH1 DlacZ DlacA, DnanKETA, |
| | DmelA, DwcaJ, DmdoH wcaF::Plac |
| Strain X1 | MDO, x1 lacY_mut1-PglpF, x1 galT-PglpF |
| Strain X2 | MDO, x1 lacY_mut2-PglpF, x1 galT-PglpF |
| Strain X3 | MDO, x1 lacY_mut6-PglpF, x1 galT-PglpF |
| Strain X4 | MDO, x1 lacY_mut7-PglpF, x1 galT-PglpF |
| Strain X5 | MDO, x1 lacY_mut8-PglpF, x1 galT-PglpF |
| Strain X6 | MDO, x1 lacY_mut9-PglpF, x1 galT-PglpF |
| Strain X7 | MDO, x1 lacY_mut10-PglpF, x1 galT-PglpF |
| Strain X8 | MDO, x1 lacY_mut11-PglpF, x1 galT-PglpF |
| Strain X9 | MDO, x1 lacY_mut1-PglpF, x1 galTK-PglpF |
| Strain X10 | MDO, x1 lacY_mut2-PglpF, x1 galTK-PglpF |
| Strain X11 | MDO, x1 lacY_mut6-PglpF, x1 galTK-PglpF |
| Strain X12 | MDO, x1 lacY_mut7-PglpF, x1 galTK-PglpF |
| Strain X13 | MDO, x1 lacY_mut8-PglpF, x1 galTK-PglpF |
| Strain X14 | MDO, x1 lacY_mut9-PglpF, x1 galTK-PglpF |
| Strain X15 | MDO, x1 lacY_mut10-PglpF, x1 galTK-PglpF |
| Strain X16 | MDO, x1 lacY_mut11-PglpF, x1 galTK-PglpF |

### Strain Performance Testing

The import of LNT-II and specifically the production of the tetrasaccharide LNT or LNnT can be measured by applying a fed-batch cultivation protocol, where glucose is being slowly released by invertase-mediated enzymatic cleavage of the provided sucrose. In such strain performance assay, LNT-II is also added to the medium as acceptor oligosaccharide for the generation of LNT or LNnT. A detailed protocol of the assay is provided below. Samples generated using the protocol below can be analyzed by HPLC to quantify the levels of the formed oligosaccharide.

The strains described in the present example can be screened in 96 deep well plates using a 4-day protocol. During the first 24 hours, precultures are grown to high densities and subsequently transferred to a medium that allows the induction of gene expression and product formation. More specifically, during day 1, fresh precultures are prepared using a basal minimal medium supplemented with magnesium sulphate, thiamine and glucose. The precultures are incubated for 24 hours at 34° C. and 1000 rpm shaking and then further transferred to a new basal minimal medium (BMM, pH 7.5) to start the main culture. The new BMM is supplemented with magnesium sulphate, thiamine, a bolus of 0.1 g/L glucose, IPTG (50 mg/mL) and a bolus of between 20 and 200 g/L, LNT-II. Moreover, 37.5 g/L sucrose is provided as carbon source, accompanied by the addition of sucrose hydrolase (invertase), so that glucose is released at a rate suitable for carbon limited growth. The main cultures are incubated for 72 hours at 28° C. and 1000 rpm shaking.

### Example 2: Import of LNT by E. coli DH1 K12 Cells to Produce Complex HMOs with an LNT-Core

### Strain Design

In the present example a modified E. coli DH1 K12 strain is engineered to produce pentasaccharides including lacto-N-fucopentaose I (LNFP-1), lacto-N-fucopentaose II (LNFP-II), lacto-N-fucopentaose V (LNFP-V), sialyl-lacto-N-tetraose a (LST-a) and sialyl-lacto-N-tetraose b (LST-b), or even the hexasaccharides lacto-N-difucohexaose I (LNDFH-1), lacto-N-difucohexaose II (LNDFH-II), disialyllacto-N-tetraose (DSLNT) and lacto-N-hexaose (LNH). LNT can be added to the culture medium of a strain that expresses an MFS or ABC transporter protein originating from a Gram+ bacteria, and in particular members of the Bifidobacterium, Roseburia and Eubacterium species (Table 5) and an α-1,2- and/or α-1,4-, or α-1,3- or α-1,3/4-fucosyltransferase, or an α-2,3- and/or α-2,6-sialyltransferase, or a β-1,6-acetylglucosaminyl-transferase and/or a β-1,4-galactosyltransferase (Table 6) enabling the transfer of one or two glycosyl residues to the LNT acceptor oligosaccharide imported into the cell by the transporter.

TABLE 5
List of transporter proteins of Gram+ origin that can be expressed
for the import of the precursor oligosaccharide molecule LNT. The original
loci encoding ABC transporters are composed of three to four genes. For
ease of reference each transporter has been given a transporter ID (TP ID)

| | | | | SEQ | Expected | |
|---|---|---|---|---|---|---|
| TP | TP | | | ID | oligosaccharide | |
| ID | type | Gene Name | GenBank ID | NO: | transport | Origin |
| 1 | MFS | BIon_0247 | ACJ51375.1 | 2 | LN(n)T | B. infantis ATCC |
| | | | | | | 15697 = JCM 1222 |
| 2 | MFS | BIon_0431 | ACJ51548.1 | 6 | oligosaccharide | B. infantis ATCC |
| | | | | | | 15697 = JCM 1222 |
| 3 | MFS | BIon_0788 | ACJ51891.1 | 7 | oligosaccharide | B. infantis ATCC |
| | | | | | | 15697 = JCM 1222 |
| 4 | MFS | BIon_2400 | ACJ53455.1 | 27 | oligosaccharide | B. infantis ATCC |
| | | | | | | 15697 = JCM 1222 |
| 5 | ABC | BIon_2341 | ACJ53399.1 | 20 | LN(n)T, tri- | B. infantis ATCC |
| | | | | | lactosamine, di- | 15697 = JCM 1222 |
| | | BIon_2342 | ACJ53400.1 | 21 | lactosamine, | B. infantis ATCC |
| | | | | | branched tri- | 15697 = JCM 1222 |
| | | BIon_2343 | ACJ53401.1 | 22 | lactosamine | B. infantis ATCC |
| | | | | | | 15697 = JCM 1222 |
| | | BIon_2344 | ACJ53402.1 | 23 | | B. infantis ATCC |
| | | | | | | 15697 = JCM 1222 |
| 6 | ABC | BIon_2345 | ACJ53403.1 | 24 | LN(n)T, tri- | B. infantis ATCC |
| | | | | | lactosamine, di- | 15697 = JCM 1222 |
| | | BIon_2346 | ACJ53404.1 | 25 | lactosamine, | B. infantis ATCC |
| | | | | | branched tri- | 15697 = JCM 1222 |
| | | BIon_2347 | ACJ53405.1 | 26 | lactosamine, | B. infantis ATCC |
| | | | | | dimeric Lewis X | 15697 = JCM 1222 |
| 7 | ABC | BIon_0883 | ACJ51983.1 | 8 | LN(n)T, LNB, | B. infantis ATCC |
| | | | | | GNB, Lewis a, | 15697 = JCM 1222 |
| | | BIon_0884 | ACJ51984.1 | 9 | Lewis Le^{x}, | B. infantis ATCC |
| | | | | | type 1 blood | 15697 = JCM 1222 |
| | | BIon_0885 | ACJ51985.1 | 10 | group H- | B. infantis ATCC |
| | | | | | trisaccharide | 15697 = JCM 1222 |
| | | BIon_0886 | ACJ51986.1 | 11 | | B. infantis ATCC |
| | | | | | | 15697 = JCM 1222 |
| 8 | ABC | BIon_2177 | ACJ53238.1 | 15 | LN(n)T, LNB, | B. infantis ATCC |
| | | | | | GNB, dimeric | 15697 = JCM 1222 |
| | | BIon_2176 | ACJ53237.1 | 14 | LNB, LNO, LNFO, | B. infantis ATCC |
| | | | | | LST-b, galactosyl- | 15697 = JCM 1222 |
| | | BIon_2175 | ACJ53236.1 | 13 | lactosamine | B. infantis ATCC |
| | | | | | | 15697 = JCM 1222 |
| 9 | ABC | RHOM_04095 | AEN95941.1 | 35 | LNT | R. hominis A2-183 |
| | | RHOM_04100 | AEN95942.1 | 36 | | R. hominis A2-183 |
| | | RHOM_04105 | AEN95943.1 | 37 | | R. hominis A2-183 |
| 10 | ABC | HMPREF0373_02960 | ERK42291.1 | 38 | LNT | E. ramulus ATCC |
| | | | | | | 29099 |
| | | HMPREF0373_02961 | ERK42292.1 | 39 | | E. ramulus ATCC |
| | | | | | | 29099 |
| | | HMPREF0373_02962 | ERK42293.1 | 40 | | E. ramulus ATCC |
| | | | | | | 29099 |
| 11 | ABC | Bbr_0527/IntP1 | ABE95224.1 | 28 | LN(n)T, LNB | B. breve UCC2003 |
| | | Bbr_0528/IntP2 | ABE95225.1 | 29 | | B. breve UCC2003 |
| | | Bbr_0530/IntS | ABE95227.1 | 30 | | B. breve UCC2003 |
| | | Bbr_0531 | ABE95228.1 | 50 | | B. breve UCC2003 |

LNB: lacto-N-biose,
GNB: galacto-N-biose,
LNO: iso-lacto-N-octaose,
LNFO: iso-lacto-N-fucosyl-octaose

TABLE 6
List of glycosyltransferases that can be expressed by a complex HMO-producing strain
that also expresses at least one LNT importer of Gram+ origin (strain Y).
The table indicated the expected complex HMO product to be produced by the glycosyltransferase.
Some products require that two glycosyl transferases are expressed

| Protein | | | | Complex HMO |
|---|---|---|---|---|
| Name | Enzyme Type | GenBank ID | Origin | product |
| FutC | α-1,2-fucosyltransferase | WP_080473865.1 | Helicobacter | LNFP-I |
| | | | pylori ATCC | |
| | | | 26695 | |
| Smob | α-1,2-fucosyltransferase | WP_126455392.1 | Sulfuriflexus | LNFP-I |
| | | | mobilis | |
| FucT2_HpUA802 | α-1,2-fucosyl-transferase | AAC99764.1 | Helicobacter | LNFP-I, |
| | | | pylori | LNDFH-I |
| FucT2_EcO126t | α-1,2-fucosyl-transferase | ABE98421.1 | Escherichia coli | LNFP-I, |
| | | | | LNDFH-I |
| FucT2_Hm12198 | α-1,2-fucosyl-transferase | CBG40460.1 | Helicobacter | LNFP-I, |
| | | | mustelae 12198 | LNDFH-I |
| FucT2_Pm9515 | α-1,2-fucosyl-transferase | ABM71599.1 | Brochlorococcus | LNFP-I, |
| | | | marinus str. MIT | LNDFH-I |
| | | | 9515 | |
| FucT2_HpF57 | α-1,2-fucosyl-transferase | BAJ59215.1 | Helicobacter | LNDFH-I, |
| | | | pylori F57 | LNDFH-I |
| Mtun | α-1,2-fucosyl-transferase | WP_031437198.1 | Methylobacter | LNFP-I, |
| | | | tundripaludum | LNDFH-I |
| FucTIII | α-1,3/4-fucosyltransferase | AY450598.1 | Helicobacter | LNFP-II, |
| | | | pylori strain | LNFP-V, |
| | | | DSM 6709 | LNDFH-II |
| FutA | α-1,3-fucosyltransferase | WP_000487428.1 | Helicobacter | LNFP-V, |
| | | | pylori ATCC | LNDFH-II |
| | | | 26695 | |
| FucT | α-1,3-fucosyltransferase | AAB81031.1 | Helicobacter | LNFP-V, |
| | | | pylori NCTC | LNDFH-II |
| | | | 11639 | |
| FucT6_3_Bf | α-1,3-fucosyltransferase | CAH09151.1 | Bacteroides | LNFP-V, |
| | | | fragilis NCTC | LNDFH-II |
| | | | 9343 | |
| FucT7_3_Bf | α-1,3-fucosyltransferase | CAH09495.1 | Bacteroides | LNFP-V, |
| | | | fragilis NCTC | LNDFH-II |
| | | | 9343 | |
| FucT_3_Am | α-1,3-fucosyltransferase | ACD04596.1 | Akkermansia | LNFP-V, |
| | | | muciniphila ATCC | LNDFH-II |
| | | | BAA-835 | |
| MAMA_R764 | α-1,3-fucosyltransferase | AGC02224.1 | Acanthamoeba | LNFP-V, |
| | | | polyphaga | LNDFH-II |
| | | | moumouvirus | |
| Mg791 | α-1,3-fucosyltransferase | AEQ33441.1 | Megavirus | LNFP-V, |
| | | | chiliensis | LNDFH-II |
| Moumou_00703 | α-1,3-fucosyltransferase | YP_007354660.1 | Acanthamoeba | LNFP-V, |
| | | | polyphaga | LNDFH-II |
| | | | moumouvirus | |
| Ccol | α-2,3-sialyltransferase | WP_075498955.1 | Campylobacter | LST-a |
| | | | coli | |
| PM70 | α-2,3-sialyltransferase | AAK03258.1 | Pasteurella | LST-a |
| | | | multocida subsp. | |
| | | | multocida str. | |
| | | | Pm70 | |
| Pts6-119 | α-2,6-sialyltransferase | AB500947.1 | Photobacterium | LST-b |
| | | | leiognathi | |
| | | | JT-SHIZ-119 | |
| Pts6-224 | α-2,6-sialyltransferase | BAF92026.1 | Photobacterium | LST-b |
| | | | sp JT-ISH-224 | |
| Ccol | α-2,3-sialyltransferase | WP_075498955.1 | Campylobacter | DSLNT |
| | | | coli | |
| Pts6-224 | α-2,6-sialyltransferase | BAF92026.1 | Photobacterium | |
| | | | sp JT-ISH-224 | |
| Csp2 | β-1,6-N- | WP_22844786.1 | Chryseobacterium | LNH |
| | acetylglucosaminyltransferase | | sp. KBW03 | |

### Strain Construction

During strain construction, the genome of the platform strain MO that is well-suited for HMO production (WO2019/123324, see also table 4 in example 1) can be engineered using methods that are well-known in the art (e.g., gene gorging by Herring & Blattner 2004 Conditional lethal amber mutations in essential Escherichia coli genes. J Bacteriol 186:2673-2681), so that a few novel expression cassettes are integrated at non-essential genomic loci: a) an expression cassette containing a constitutive or inducible promoter (e.g., Plac, PglpF, Ptac, Pbad, PosmY) controlling the expression of the coding sequence of a gene encoding an ABC or MFS transporter from Gram+ bacteria (Table 5) and a terminator sequence (e.g., T1), b) an expression cassette containing a constitutive or inducible promoter (e.g., Plac, PglpF, Ptac, Pbad, PosmY), controlling the expression of the coding sequence of a gene encoding a given glycosyltransferase (Table 6) and a terminator sequence (e.g., T1) and optionally c) an expression cassette containing a constitutive or inducible promoter (e.g., Plac, PglpF, Ptac, Pbad, PosmY), controlling the expression of the coding sequence of a gene encoding another glycosyltransferase (Table 6) and a terminator sequence (e.g., T). The resulting strain is hereby denoted as strain Y (Table 7).

Exemplary genotypes for this example are provided in Table 7 below.

TABLE 7
Exemplary genotypes of strains described in the present example

| | | Complex HMO |
|---|---|---|
| Strain ID | Genotype | product |
| Strain Y1 | MDO, x1 TP1-PglpF, x1 smob-PglpF | LNFP-I |
| Strain Y2 | MDO, x1 TP2-PglpF, x1 smob-PglpF | LNFP-I |
| Strain Y3 | MDO, x1 TP3-PglpF, x1 smob-PglpF | LNFP-I |
| Strain Y4 | MDO, x1 TP4-PglpF, x1 smob-PglpF | LNFP-I |
| Strain Y5 | MDO, x1 TP5-PglpF, x1 smob-PglpF | LNFP-I |
| Strain Y6 | MDO, x1 TP6-PglpF, x1 smob-PglpF | LNFP-I |
| Strain Y7 | MDO, x1 TP7-PglpF, x1 smob-PglpF | LNFP-I |
| Strain Y8 | MDO, x1 TP8-PglpF, x1 smob-PglpF | LNFP-I |
| Strain Y9 | MDO, x1 TP9-PglpF, x1 smob-PglpF | LNFP-I |
| Strain Y10 | MDO, x1 TP10-PglpF, x1 smob-PglpF | LNFP-I |
| Strain Y11 | MDO, x1 TP11-PglpF, x1 smob-PglpF | LNFP-I |
| Strain Y12 | MDO, x1 TP1-PglpF, x1 futA-PglpF | LNFP-V, LNDFH-II |
| Strain Y13 | MDO, x1 TP2-PglpF, x1 futA-PglpF | LNFP-V, LNDFH-II |
| Strain Y14 | MDO, x1 TP3-PglpF, x1 futA-PglpF | LNFP-V, LNDFH-II |
| Strain Y15 | MDO, x1 TP4-PglpF, x1 futA-PglpF | LNFP-V, LNDFH-II |
| Strain Y16 | MDO, x1 TP5-PglpF, x1 futA-PglpF | LNFP-V, LNDFH-II |
| Strain Y17 | MDO, x1 TP6-PglpF, x1 futA-PglpF | LNFP-V, LNDFH-II |
| Strain Y18 | MDO, x1 TP7-PglpF, x1 futA-PglpF | LNFP-V, LNDFH-II |
| Strain Y19 | MDO, x1 TP8-PglpF, x1 futA-PglpF | LNFP-V, LNDFH-II |
| Strain Y20 | MDO, x1 TP9-PglpF, x1 futA-PglpF | LNFP-V, LNDFH-II |
| Strain Y21 | MDO, x1 TP10-PglpF, x1 futA-PglpF | LNFP-V, LNDFH-II |
| Strain Y22 | MDO, x1 TP11-PglpF, x1 futA-PglpF | LNFP-V, LNDFH-II |

### Strain Performance Testing

The import of LNT and specifically the production of penta- or hexa-saccharides of interest can be measured by applying a fed-batch cultivation protocol, where glucose is being slowly released by invertase-mediated enzymatic cleavage of the provided sucrose. In such strain performance assay, LNT is also added to the medium. A detailed protocol of the assay is provided below. Samples generated using the protocol below can be analyzed by HPLC to quantify the levels of the formed oligosaccharides.

The strains described in the present example can be screened in 96 deep well plates using a 4-day protocol. During the first 24 hours, precultures are grown to high densities and subsequently transferred to a medium that allows the induction of gene expression and product formation. More specifically, during day 1, fresh precultures are prepared using a basal minimal medium supplemented with magnesium sulphate, thiamine and glucose. The precultures are incubated for 24 hours at 34° C. and 1000 rpm shaking and then further transferred to a new basal minimal medium (BMM, pH 7.5) to start the main culture. The new BMM is supplemented with magnesium sulphate, thiamine, a bolus of 0.1 g/L glucose, IPTG (50 mg/mL) and a bolus of between 20 and 200 g/L LNT. Moreover, 37.5 g/L sucrose is provided as carbon source, accompanied by the addition of sucrose hydrolase (invertase), so that glucose is released at a rate suitable for carbon-limited growth. The main cultures are incubated for 72 hours at 28° C. and 1000 rpm shaking.

### Example 3: Import of LNnT by E. coli DH1 K12 Cells to Produce Complex HMOs with an LNnT-Core

### Strain Design

In the present example a modified E. coli DH1 K12 strain is engineered to produce pentasaccharides including lacto-N-fucopentaose III (LNFP-III), lacto-N-fucopentaose VI (LNFP-VI) and sialyl-lacto-N-tetraose c (LST-c), or even the hexasaccharides lacto-N-difucohexaose III (LNDFH-III), p-lacto-N-neohexaose (pLNnH), p-lacto-N-hexaose I (pLNH-1) and lacto-N-neohexaose (LNnH). LNnT can be added to the culture medium of a strain that expresses an MFS or ABC transporter protein originating from a Gram+ bacteria, and in particular members of the Bifidobacterium species (Table 8) and an α-1,3- or α-1,3/4-fucosyltransferase, or an α-2,6-sialyltransferase, or a β-1,6-acetylglucosaminyl-transferase and a β-1,4-galactosyltransferase, or a β-1,3-acetylglucosaminyl-transferase and a β-1,4-galactosyltransferase, or a β-1,3-acetylglucosaminyl-transferase and a β-1,3-galactosyltransferase (Table 9) enabling the transfer of one or two glycosyl residues to the LNnT acceptor oligosaccharide imported into the cell by the transporter.

TABLE 8
List of transporter proteins of Gram+ origin that can be expressed
for the import of the precursor oligosaccharide molecule LNnT. The
ABC transporters are composed of three to four genes. For ease of
reference each transporter has been given a transporter ID (TP ID)

| | | | | SEQ | Expected | |
|---|---|---|---|---|---|---|
| TP | TP | | | ID | oligosaccharide | |
| ID | type | Gene Name | GenBank ID | NO: | transport | Origin |
| 1 | MFS | BIon_0247 | ACJ51375.1 | 2 | LN(n)T | B. infantis ATCC |
| | | | | | | 15697 = JCM 1222 |
| 2 | MFS | BIon_0431 | ACJ51548.1 | 6 | oligosaccharide | B. infantis ATCC |
| | | | | | | 15697 = JCM 1222 |
| 3 | MFS | BIon_0788 | ACJ51891.1 | 7 | oligosaccharide | B. infantis ATCC |
| | | | | | | 15697 = JCM 1222 |
| 4 | MFS | BIon_2400 | ACJ53455.1 | 27 | oligosaccharide | B. infantis ATCC |
| | | | | | | 15697 = JCM 1222 |
| 5 | ABC | BIon_2341 | ACJ53399.1 | 20 | LN(n)T, tri- | B. infantis ATCC |
| | | | | | lactosamine, di- | 15697 = JCM 1222 |
| | | BIon_2342 | ACJ53400.1 | 21 | lactosamine, | B. infantis ATCC |
| | | | | | branched tri- | 15697 = JCM 1222 |
| | | BIon_2343 | ACJ53401.1 | 22 | lactosamine | B. infantis ATCC |
| | | | | | | 15697 = JCM 1222 |
| | | BIon_2344 | ACJ53402.1 | 23 | | B. infantis ATCC |
| | | | | | | 15697 = JCM 1222 |
| 6 | ABC | BIon_2345 | ACJ53403.1 | 24 | LN(n)T, tri- | B. infantis ATCC |
| | | | | | lactosamine, di- | 15697 = JCM 1222 |
| | | BIon_2346 | ACJ53404.1 | 25 | lactosamine, | B. infantis ATCC |
| | | | | | branched tri- | 15697 = JCM 1222 |
| | | BIon_2347 | ACJ53405.1 | 26 | lactosamine, | B. infantis ATCC |
| | | | | | dimeric Lewis X | 15697 = JCM 1222 |
| 7 | ABC | BIon_0883 | ACJ51983.1 | 8 | LN(n)T, LNB, | B. infantis ATCC |
| | | | | | GNB, Lewis a, | 15697 = JCM 1222 |
| | | BIon_0884 | ACJ51984.1 | 9 | Lewis Le^{x}, type | B. infantis ATCC |
| | | | | | 1 blood group | 15697 = JCM 1222 |
| | | BIon_0885 | ACJ51985.1 | 10 | H-trisaccharide | B. infantis ATCC |
| | | | | | | 15697 = JCM 1222 |
| | | BIon_0886 | ACJ51986.1 | 11 | | B. infantis ATCC |
| | | | | | | 15697 = JCM 1222 |
| 8 | ABC | BIon_2177 | ACJ53238.1 | 15 | LN(n)T, LNB, | B. infantis ATCC |
| | | | | | GNB, dimeric | 15697 = JCM 1222 |
| | | BIon_2176 | ACJ53237.1 | 14 | LNB, LNO, | B. infantis ATCC |
| | | | | | LNFO, LST-b, | 15697 = JCM 1222 |
| | | BIon_2175 | ACJ53236.1 | 13 | galactosyl- | B. infantis ATCC |
| | | | | | lactosamine | 15697 = JCM 1222 |
| 11 | ABC | Bbr_0527/IntP1 | ABE95224.1 | 28 | LN(n)T, LNB | B. breve UCC2003 |
| | | Bbr_0528/IntP2 | ABE95225.1 | 29 | | B. breve UCC2003 |
| | | Bbr_0530/IntS | ABE95227.1 | 30 | | B. breve UCC2003 |
| | | Bbr_0531 | ABE95228.1 | 50 | | B. breve UCC2003 |
| 12 | ABC | Bbr_1554/nahS | ABE96225.1 | 31 | LNnT | B. breve UCC2003 |
| | | Bbr_1558 | ABE96228.1 | 32 | | B. breve UCC2003 |
| | | Bbr_1559 | ABE96229.1 | 33 | | B. breve UCC2003 |
| | | Bbr_1560 | ABE96230.1 | 34 | | B. breve UCC2003 |

LNB: lacto-N-biose,
GNB: galacto-N-biose,
LNO: iso-lacto-N-octaose,
LNFO: iso-lacto-N-fucosyl-octaose

TABLE 9
List of glycosyltransferases that can be expressed by a complex HMO-producing strain
that also expresses at least one LNnT importer of Gram+ origin (strain Z).
The table indicates the expected complex HMO product to be produced by the glycosyltransferase.
Some products require that two glycosyl transferases are expressed

| | | | | Complex |
|---|---|---|---|---|
| Protein | | | | HMO |
| Name | Enzyme Type | GenBank ID | Origin | product |
| FucT | α-1,3-fucosyltransferase | AAB81031.1 | Helicobacter | LNFP-III, |
| | | | pylori NCTC | LNDFH-III |
| | | | 11639 | |
| FutA | α-1,3-fucosyltransferase | WP_000487428.1 | Helicobacter | LNFP-VI, |
| | | | pylori ATCC | LNDFH-III |
| | | | 26695 | |
| CafC | α-1,3-fucosyltransferase | WP_007483358.1 | Bacteroides | LNDFH-III |
| | | | nordii | |
| HAC1268 | α-2,6-sialyltransferase | STP03859.1 | Helicobacter | LST-c |
| | | | acinonychis str. | |
| | | | Sheeba | |
| Csp2 | β-1,6-N- | WP_22844786.1 | Chryseobacterium | LNnH |
| | acetylglucosaminyltransferase | | sp. KBW03 | |
| GalT | β-1,4-galactosyl transferase | WP_001262061.1 | Helicobacter | |
| | | | pylori | |
| LgtA | β-1,3-N- | WP_033911473.1 | Neisseria | pLNnH |
| | acetylglucosaminyltransferase | | meningitidis | |
| | | | 053442 | |
| LgtA | β-1,3-N- | WP_033911473.1 | Neisseria | pLNH-I |
| | acetylglucosaminyltransferase | | meningitidis | |
| | | | 053442 | |
| GalTK | β-1,3-galactosyl transferase | WP_111735921.1 | Helicobacter | |
| | | | pylori ATCC | |
| | | | 43504 | |

### Strain Construction

During strain construction, the genome of the platform strain MOO that is well-suited for HMO production (WO2019/123324, see also table 4 in example 1) can be engineered using methods that are well-known in the art (e.g., gene gorging by Herring & Blattner 2004 Conditional lethal amber mutations in essential Escherichia coli genes. J Bacteriol 186:2673-2681), so that a few novel expression cassettes are integrated at non-essential genomic loci: a) an expression cassette containing a constitutive or inducible promoter (e.g., Plac, PglpF, Ptac, Pbad, PosmY) controlling the expression of the coding sequence of a gene encoding an ABC or MFS transporter from Gram+ bacteria (Table 8) and a terminator sequence (e.g., T1), b) an expression cassette containing a constitutive or inducible promoter (e.g., Plac, PglpF, Ptac, Pbad, PosmY), controlling the expression of the coding sequence of a gene encoding a given glycosyltransferase (Table 9) and a terminator sequence (e.g., T1) and optionally c) an expression cassette containing a constitutive or inducible promoter (e.g., Plac, PglpF, Ptac, Pbad, PosmY), controlling the expression of the coding sequence of a gene encoding another glycosyltransferase (Table 9) and a terminator sequence (e.g., T1). The resulting strain is hereby denoted as strain Z (Table 10).

Exemplary genotypes for this example are provided in Table 10 below.

TABLE 10
Exemplary genotypes of strains relevant for the present example

| Strain | | Complex HMO |
|---|---|---|
| ID | Genotype | product |
| Strain Z1 | MDO, x1 TP1-PglpF, x1 fucT-PglpF | LNFP-III, LNDFH-III |
| Strain Z2 | MDO, x1 TP2-PglpF, x1 fucT-PglpF | LNFP-III, LNDFH-III |
| Strain Z3 | MDO, x1 TP3-PglpF, x1 fucT-PglpF | LNFP-III, LNDFH-III |
| Strain Z4 | MDO, x1 TP4-PglpF, x1 fucT-PglpF | LNFP-III, LNDFH-III |
| Strain Z5 | MDO, x1 TP5-PglpF, x1 fucT-PglpF | LNFP-III, LNDFH-III |
| Strain Z6 | MDO, x1 TP6-PglpF, x1 fucT-PglpF | LNFP-III, LNDFH-III |
| Strain Z7 | MDO, x1 TP7-PglpF, x1 fucT-PglpF | LNFP-III, LNDFH-III |
| Strain Z8 | MDO, x1 TP8-PglpF, x1 fucT-PglpF | LNFP-III, LNDFH-III |
| Strain Z9 | MDO, x1 TP11-PglpF, x1 fucT-PglpF | LNFP-III, LNDFH-III |
| Strain Z10 | MDO, x1 TP12-PglpF, x1 fucT-PglpF | LNFP-III, LNDFH-III |
| Strain Z11 | MDO, x1 TP1-PglpF, x1 futA-PglpF | LNFP-VI, LNDFH-III |
| Strain Z12 | MDO, x1 TP2-PglpF, x1 futA-PglpF | LNFP-VI, LNDFH-III |
| Strain Z13 | MDO, x1 TP3-PglpF, x1 futA-PglpF | LNFP-VI, LNDFH-III |
| Strain Z14 | MDO, x1 TP4-PglpF, x1 futA-PglpF | LNFP-VI, LNDFH-III |
| Strain Z15 | MDO, x1 TP5-PglpF, x1 futA-PglpF | LNFP-VI, LNDFH-III |
| Strain Z16 | MDO, x1 TP6-PglpF, x1 futA-PglpF | LNFP-VI, LNDFH-III |
| Strain Z17 | MDO, x1 TP7-PglpF, x1 futA-PglpF | LNFP-VI, LNDFH-III |
| Strain Z18 | MDO, x1 TP8-PglpF, x1 futA-PglpF | LNFP-VI, LNDFH-III |
| Strain Z19 | MDO, x1 TP11-PglpF, x1 futA-PglpF | LNFP-VI, LNDFH-III |
| Strain Z20 | MDO, x1 TP12-PglpF, x1 futA-PglpF | LNFP-VI, LNDFH-III |

### Strain Performance Testing

The import of LNT and specifically the production of penta- or hexa-saccharides of interest can be measured by applying a fed-batch cultivation protocol, where glucose is being slowly released by invertase-mediated enzymatic cleavage of the provided sucrose. In such strain performance assay, LNnT is also added to the medium. A detailed protocol of the assay is provided below. Samples generated using the protocol below can be analyzed by HPLC to quantify the levels of the formed oligosaccharides.

The strains described in the present example can be screened in 96 deep well plates using a 4-day protocol. During the first 24 hours, precultures are grown to high densities and subsequently transferred to a medium that allows the induction of gene expression and product formation. More specifically, during day 1, fresh precultures are prepared using a basal minimal medium supplemented with magnesium sulphate, thiamine and glucose. The precultures are incubated for 24 hours at 34° C. and 1000 rpm shaking and then further transferred to a new basal minimal medium (BMM, pH 7.5) to start the main culture. The new BMM is supplemented with magnesium sulphate, thiamine, a bolus of 0.1 g/L glucose, IPTG (50 mg/mL) and a bolus of between 20 and 200 g/L LNnT. Moreover, 37.5 g/L sucrose is provided as carbon source, accompanied by the addition of sucrose hydrolase (invertase), so that glucose is released at a rate suitable for carbon-limited growth. The main cultures are incubated for 72 hours at 28 2C and 1000 rpm shaking.

### Example 4: Import of 2′FL, 3FL and/or LNFP-1 by E. coli DH K12 Cells to Produce Complex HMOs with an FL-Core

### Strain Design

In the present example a modified E. coli DH1 K12 strain is engineered to produce the tetrasaccharides difucosyllactose (DFL) and 3-fucosyl-3′-sialyllactose (FSL), or even the hexa-saccharide lacto-N-difucohexaose I (LNDFH-2). 2-FL, 3FL and/or LNFP-I can be added to the culture medium of a strain that expresses an MFS or ABC transporter protein originating from a Gram+ bacteria, and in particular members of the Bifidobacterium species (Table 11) and an a-1,2-, or α-1,4-, or α-1,3- or α-1,3/4-fucosyltransferase, or an α-2,3-sialyltransferase (Table 12) enabling the transfer of a single glycosyl residue to the 2-FL, 3FL and/or LNFP-2 acceptor oligosaccharide imported into the cell by the transporter.

TABLE 11
List of transporter proteins of Gram+ origin that can be expressed for the
import of a fucosylated precursor oligosaccharide molecule comprising of 3 to
5 monosaccharides. The ABC transporters are composed of three to four genes.
For ease of reference each transporter has been given a transporter ID (TP ID)

| | | | | SEQ | Expected | |
|---|---|---|---|---|---|---|
| TP | TP | | | ID | oligosaccharide | |
| ID | type | Gene Name | GenBank ID | NO: | transport | Origin |
| 2 | MFS | BIon_0431 | ACJ51548.1 | 6 | oligosaccharide | B. infantis ATCC |
| | | | | | | 15697 = JCM 1222 |
| 3 | MFS | BIon_0788 | ACJ51891.1 | 7 | oligosaccharide | B. infantis ATCC |
| | | | | | | 15697 = JCM 1222 |
| 4 | MFS | BIon_2400 | ACJ53455.1 | 27 | oligosaccharide | B. infantis ATCC |
| | | | | | | 15697 = JCM 1222 |
| 13 | MFS | BIon_0962 | ACJ52061.1 | 12 | Fucosylated-HMO | B. infantis ATCC |
| | | | | | | 15697 = JCM 1222 |
| 14 | MFS | BIon_2307 | ACJ53365.1 | 19 | Fucosylated-HMO | B. infantis ATCC |
| | | | | | | 15697 = JCM 1222 |
| 15 | ABC | BIon_0341/ | ACJ51465.1/ | 3 | 2′-FL, 3-FL, DFL, | B. infantis ATCC |
| | | BIon_2204 | ACJ53264.1 | 18 | LNFP-I | 15697 = JCM 1222 |
| | | BIon_0342/ | ACJ51466.1/ | 4 | | B. infantis ATCC |
| | | BIon_2203 | ACJ53263.1 | 17 | | 15697 = JCM 1222 |
| | | BIon_2202 | ACJ53262.1 | 16 | | B. infantis ATCC |
| | | | | | | 15697 = JCM 1222 |
| 16 | ABC | BIon_0341/ | ACJ51465.1/ | 3 | 2′-FL, 3-FL, DFL, | B. infantis ATCC |
| | | BIon_2204 | ACJ53264.1 | 18 | LNFP-I | 15697 = JCM 1222 |
| | | BIon_0342/ | ACJ51466.1/ | 4 | | B. infantis ATCC |
| | | BIon_2203 | ACJ53263.1 | 17 | | 15697 = JCM 1222 |
| | | BIon_0343 | ACJ51467.1 | 5 | | B. infantis ATCC |
| | | | | | | 15697 = JCM 1222 |
| 17 | ABC | BBKW_1838/ | BAQ29973.1/ | 44 | 2′-FL, 3-FL, DFL, | B. kashiwanohense |
| | | BKAS_1807 | KFI63925.1 | 43 | LNFP-I | JCM 15439 |
| | | BBKW_1839/ | BAQ29974.1/ | 45 | | B. kashiwanohense |
| | | BKAS_1806 | KFI63924.1 | 52 | | JCM 15439 |
| | | BBKW_1840/ | BAQ29975.1/ | 46 | | B. kashiwanohense |
| | | BKAS_1805 | KFI63923.1 | 41 | | JCM 15439 |
| 18 | ABC | BIFPSEUDO_03224/ | BAR04453.1 | 47 | 2′-FL, 3-FL, DFL, | B. |
| | | BBPC_1775 | | | LNFP-I | pseudocatenulatum |
| | | | | | | JCM 1200 |
| | | BIFPSEUDO_03225/ | BAR04454.1 | 48 | | B. |
| | | BBPC_1776 | | | | pseudocatenulatum |
| | | | | | | JCM 1200 |
| | | BIFPSEUDO_03226/ | BAR04455.1 | 49 | | B. |
| | | BBPC_1777 | | | | pseudocatenulatum |
| | | | | | | JCM 1200 |

TABLE 12
List of glycosyltransferases that can be expressed by a complex
HMO-producing strain that also expresses at least one FL (2′FL,
3FL, LNFP-I) importer of Gram+ origin (strain T)

| | | | | Complex |
|---|---|---|---|---|
| Protein | | | | HMO |
| Name | Enzyme Type | GenBank ID | Origin | product |
| FutC | α-1,2- | WP_080473865.1 | Helicobacter pylori | DFL |
| | fucosyltransferase | | ATCC 26695 | |
| wbgL | α-1,2- | ADN43847 | E. coli O126 | DFL |
| | fucosyltransferase | | | |
| FutA | α-1,3- | WP_000487428.1 | Helicobacter pylori | DFL |
| | fucosyltransferase | | ATCC 26695 | |
| FucT | α-1,3- | AAB81031 | Helicobacter pylori | DFL |
| | fucosyltransferase | | NCTC 11639 | |
| FucTIII | α-1,3/4- | AY450598.1 | Helicobacter pylori | LNDFH-I |
| | fucosyltransferase | | strain DSM 6709 | |
| Ccol | α-2,3- | WP_075498955.1 | Campylobacter coli | FSL |
| | sialyltransferase | | | |

### Strain Construction

During strain construction, the genome of the platform strain MOO that is well-suited for HMO production (WO2019/123324, see also table 4 in example 1) can be engineered using methods that are well-known in the art (e.g., gene gorging by Herring & Blattner 2004 Conditional lethal amber mutations in essential Escherichia coli genes. J Bacteriol 186:2673-2681), so that a few novel expression cassettes are integrated at non-essential genomic loci: a) an expression cassette containing a constitutive or inducible promoter (e.g., Plac, PglpF, Ptac, Pbad, PosmY) controlling the expression of the coding sequence of a gene encoding an ABC or MFS transporter from Gram+ bacteria (Table 11) and a terminator sequence (e.g., T1), b) an expression cassette containing a constitutive or inducible promoter (e.g., Plac, PglpF, Ptac, Pbad, PosmY), controlling the expression of the coding sequence of a gene encoding a given glycosyltransferase (Table 12) and a terminator sequence (e.g., T1) and optionally c) an expression cassette containing a constitutive or inducible promoter (e.g., Plac, PglpF, Ptac, Pbad, PosmY), controlling the expression of the coding sequence of a gene encoding another glycosyltransferase (Table 12) and a terminator sequence (e.g., T1). The resulting strain is hereby denoted as strain T (Table 13).

Exemplary genotypes for this example are provided in Table 13 below.

TABLE 13
Exemplary genotypes of strains relevant for the present example

| Strain | | Complex HMO |
|---|---|---|
| ID | Genotype | product |
| Strain T1 | MDO, x1 TP2-PglpF, x1 futA-PglpF | DFL |
| Strain T2 | MDO, x1 TP3-PglpF, x1 futA-PglpF | DFL |
| Strain T3 | MDO, x1 TP4-PglpF, x1 futA-PglpF | DFL |
| Strain T4 | MDO, x1 TP13-PglpF, x1 futA-PglpF | DFL |
| Strain T5 | MDO, x1 TP14-PglpF, x1 futA-PglpF | DFL |
| Strain T6 | MDO, x1 TP15-PglpF, x1 futA-PglpF | DFL |
| Strain T7 | MDO, x1 TP16-PglpF, x1 futA-PglpF | DFL |
| Strain T8 | MDO, x1 TP17-PglpF, x1 futA-PglpF | DFL |
| Strain T9 | MDO, x1 TP18-PglpF, x1 futA-PglpF | DFL |
| Strain T10 | MDO, x1 TP2-PglpF, x1 futC-PglpF | DFL |
| Strain T11 | MDO, x1 TP3-PglpF, x1 futC-PglpF | DFL |
| Strain T12 | MDO, x1 TP4-PglpF, x1 futC-PglpF | DFL |
| Strain T13 | MDO, x1 TP13-PglpF, x1 futC-PglpF | DFL |
| Strain T14 | MDO, x1 TP14-PglpF, x1 futC-PglpF | DFL |
| Strain T15 | MDO, x1 TP15-PglpF, x1 futC-PglpF | DFL |
| Strain T16 | MDO, x1 TP16-PglpF, x1 futC-PglpF | DFL |
| Strain T17 | MDO, x1 TP17-PglpF, x1 futC-PglpF | DFL |
| Strain T18 | MDO, x1 TP18-PglpF, x1 futC-PglpF | DFL |
| Strain T19 | MDO, x1 TP2-PglpF, x1 fucTIII-PglpF | LNDFH-I |
| Strain T20 | MDO, x1 TP3-PglpF, x1 fucTIII-PglpF | LNDFH-I |
| Strain T21 | MDO, x1 TP4-PglpF, x1 fucTIII-PglpF | LNDFH-I |
| Strain T22 | MDO, x1 TP13-PglpF, x1 fucTIII-PglpF | LNDFH-I |
| Strain T23 | MDO, x1 TP14-PglpF, x1 fucTIII-PglpF | LNDFH-I |
| Strain T24 | MDO, x1 TP15-PglpF, x1 fucTIII-PglpF | LNDFH-I |
| Strain T25 | MDO, x1 TP16-PglpF, x1 fucTIII-PglpF | LNDFH-I |
| Strain T26 | MDO, x1 TP17-PglpF, x1 fucTIII-PglpF | LNDFH-I |
| Strain T27 | MDO, x1 TP18-PglpF, x1 fucTIII-PglpF | LNDFH-I |

### Strain Performance Testing

The import of LNT and specifically the production of penta- or hexa-saccharides of interest can be measured by applying a fed-batch cultivation protocol, where glucose is being slowly released by invertase-mediated enzymatic cleavage of the provided sucrose. In such strain performance assay, 2′-FL or 3-FL and/or LNFP-1 is also added to the medium. A detailed protocol of the assay is provided below. Samples generated using the protocol below can be analyzed by HPLC to quantify the levels of the formed oligosaccharides.

The strains described in the present example can be screened in 96 deep well plates using a 4-day protocol. During the first 24 hours, precultures are grown to high densities and subsequently transferred to a medium that allows the induction of gene expression and product formation. More specifically, during day 1, fresh precultures are prepared using a basal minimal medium supplemented with magnesium sulphate, thiamine and glucose. The precultures are incubated for 24 hours at 34° C. and 1000 rpm shaking and then further transferred to a new basal minimal medium (BMM, pH 7.5) to start the main culture. The new BMM is supplemented with magnesium sulphate, thiamine, a bolus of 0.1 g/L glucose, IPTG (50 mg/mL) and a bolus of between 20 and 200 g/L 2′-FL or 3-FL and/or LNFP-I. Moreover, 37.5 g/L sucrose is provided as carbon source, accompanied by the addition of sucrose hydrolase (invertase), so that glucose is released at a rate suitable for carbon-limited growth. The main cultures are incubated for 72 hours at 28° C. and 1000 rpm shaking.

### Example 5 Import of LNT-II by Heterologous Transporter Proteins or LacY Mutant Variants

The present example set out to test selected importers from table 1 and 2 for their ability to import LNT-II.

Genetically modified cells expressing β-1,4 galactosyltransferase GalT from Helicobacter pylori and optionally a high-copy number plasmid bearing gene(s) encoding heterologous transporter protein(s) or a LacY mutant variant were screened in a deep well assay for their ability to import LNT-II, which was substantiated by the formation of the tetrasaccharide, LNnT, at levels that were higher than the control strain (MP1, that does not express a heterologous transporter protein or a LacY mutant variant).

### Strains

The strains (genetically modified cells) constructed in the present application were based on Escherichia coli K-12 DH1 with the genotype: F⁻, λ⁻, gyrA96, recA1, relA1, endA1, thi-1, hsdR17, supE44. Additional modifications were made to the E. coli K-12 DH1 strain to generate the MDO strain with the following modifications: lacZ: deletion of 1.5 kbp, lacA: deletion of 0.5 kbp, nanKETA: deletion of 3.3 kbp, melA: deletion of 0.9 kbp, wcaJ: deletion of 0.5 kbp, mdoH: deletion of 0.5 kbp, and insertion of Plac promoter upstream of the gmd gene.

Methods of inserting gene(s) of interest into the genome of E. coli are well known to the person skilled in the art. Insertion of genetic cassettes into the E. coli chromosome can be done using gene gorging (see e.g., Herring and Blattner 2004 J. Bacteriol. 186: 2673-81 and Warming et al 2005 Nucleic Acids Res. 33(4): e36) with specific selection marker genes and screening methods.

To test the ability of at least one of each transporter protein type to import the trisaccharide LNT-II, the MDO strain was further engineered to express a β-1,4 galactosyltransferase (GalT from Helicobacter pylori, homologous to NCBI Accession nr. WP_001262061.1) and selected importer proteins of the present invention. Such MDO-derived strains should be able to produce LNnT when fed with the trisaccharide LNT-II during cultivation.

The MDO strain was modified by chromosomally integrating a single copy of the galT gene under the control of a PglpF promoter. Furthermore the cell was transformed with a high-copy (>300 copies per cell) plasmid (pUC origin of replication) bearing the gene(s) encoding a transporter of any of the above-mentioned transporter types under control of the PglpF promoter (SEQ ID NO: 70). All the heterologous gene(s) encoding for the glycosyltransferase GalT and/or a transporter protein were codon optimized for expression in E. coli. The background strain, i.e., MDO strain expressing the GalT enzyme, was named MP1. This strain was then transformed with plasmids pUC57-lacY_Y236H-PglpF-amp or pUC57-BBPC_1775-1777-PglpF-amp or pUC57-Bbr_0527-0528-0530-0531-PglpF-amp or pUC57-lacY_A177VS306T-PglpF-amp or pUC57-Blon_0962-PglpF-amp to construct the strains MP2, MP3, MP4, MP5 or MP6, respectively. The genotypes of these strains are provided in Table 15 below.

TABLE 15
Genotypes of the strains, capable of importing
LNT-II, used in the present example

| | | Importer* |
|---|---|---|
| Strain | | na SEQ |
| ref | Genotype | ID NO |
| MDO | F−λ− ΔendA1 ΔrecA1 ΔrelA1 | — |
| | ΔgyrA96 Δthi-1 glnV44 hsdR17(rK−mK−) | |
| | ΔlacZ wcaF::Plac ΔnanKETA ΔlacA | |
| | ΔmelA ΔwcaJ ΔmdoH | |
| MP1 | MDO, 1x galT-PglpF | — |
| MP2 | MP1, pUC57-lacY_Y236H-PglpF-amp | 76 |
| MP3 | MP1, pUC57- BBPC_1775-1777-PglpF-amp | 54 |
| MP4 | MP1, pUC57-Bbr_0527-0528-0530-0531-PglpF-amp | 55 |
| MP5 | MP1, pUC57-lacY_A177VS306T-PglpF-amp | 77 |
| MP6 | MP1, pUC57-BIon_0962-PglpF-amp | 67 |

*The importer proteins were expressed from a high-copy plasmid (>300 copies per cell), namely pUC57, and a relatively strong promoter, namely PglpF. The nucleic acid sequence (na) inserted into the plasmid and encoding the transporter protein is indicated as importer na. SEQ ID NO.

### Deep Well Assay

Strains MP1 to MP6 were tested for their ability to import LNT-II that was added in the cultivation medium in deep well assays. The ability to import LNT-II was confirmed by HPLC analysis of the collected cultivation samples, where LNnT measurements at levels that are higher than the strain MP1 (i.e., control strain that does not express a heterologous transporter protein or a LacY mutant variant) was indicative of introduced ability to internalize LNT-II.

The Deep well assay was performed as originally described to Lv et al (Bioprocess Biosyst Eng 20 (2016) 39:1737-1747) and optimized for the purposes of the current invention. More specifically, the strains disclosed in the present example were screened in 96 deep well plates using a 4-day protocol. During the first 24 hours, precultures were grown to high densities (OD600 up to 5) and subsequently transferred to a medium that allowed induction of gene expression and product formation. The assay was performed with replicates of four.

More specifically, during day 1, fresh precultures were prepared using a basal minimal medium (BMM) (pH 7,0) supplemented with magnesium sulphate (0.12 g/L), thiamine (0.004 g/L) and glucose (5.5 g/L). Basal Minimal medium had the following composition: NaOH (1 g/L), KOH (2.5 g/L), KH2PO4 (7 g/L), NH4H2PO4 (7 g/L), Citric acid (0.5 g/l), trace mineral solution (5 mL/L). The trace mineral stock solution contained; ZnSO4*7H2O 0.82 g/L, Citric acid 20 g/L, MnSO4*H2O 0.98 g/L, FeSO4*7H2O 3.925 g/L, CuSO4*5H2O 0.2 g/L. The pH of the Basal Minimal Medium was adjusted to 7.0 with 5 N NaOH and autoclaved. The precultures were incubated for 24 hours at 34° C. and 1000 rpm shaking and then further transferred to 0.75 mL of a new BMM (pH 7.5) to start the main culture. The new BMM was supplemented with magnesium sulphate (0.12 g/L), thiamine (0.02 g/L), a bolus of glucose solution (0.1-0.15 g/L) and a bolus of LNT-II solution (20 g/L) Moreover, a 20% stock solution of sucrose (40-45 g/L) was provided as carbon source, accompanied by the addition of a sucrose hydrolase, so that glucose was released at a rate suitable for carbon-limited growth and similar to that of a typical fed-batch fermentation process. The main cultures were incubated for 72 hours at 28° C. and 1000 rpm shaking. For the analysis of total broth, the 96 well plates were boiled at 100° C., subsequently centrifuged, and finally the supernatants were analyzed by HPLC and the concentration of LNT-II and LNnT was precisely measured and reported.

The genetically modified strains generated as described in the strain section above, expressing proteins of the Major Facilitator Superfamily (MFS) (i.e., strain MP6), or ATP-binding cassette transporters (ABC transporters) (i.e., strains MP3 and MP4) or porter proteins (i.e., mutant variants of the E. coli LacY symporter—strains MP2 and MP5) were screened in a in a fed-batch deep well assay setup as described above. The concentration of LNT-II and LNnT was precisely measured and reported. It is noteworthy that only the two above-mentioned sugars were detected, i.e., no additional compounds were formed in the chosen experimental setup, indicating that no HMO by-products were formed.

From the raw data it was observed that bacterial cells not containing any recombinant importers (control strain, MP1) form some LNnT. This could be attributed to cell lysis during cultivation (i.e., the a β-1,4 galactosyltransferase is released from the cell and performs glycosylation in the culture medium) or simply to the innate ability of E. coli cells to import low amounts of LNT-II.

To account for the LNnT production in the control cells, we initially calculated the average and standard deviations (stdv) using the LNnT concentration values reported for strains MP1 to MP6. The average LNnT values of each strain were then adjusted using the corresponding calculated stdvs for each strain. Specifically, we added the calculated stdv to the average LNnT value for the strain MP1 (to maximize the effect that background LNnT measurements have in our conclusions), and we subtracted the calculated stdv from the average LNnT value for the strains MP2, MP3, MP4, MP5 and MP6. With this calculation the beneficial effect that an importer protein has was minimized to secure that there was no overlap between the standard deviation of the control (MP1) and the results of MP2 to MP6.

Using the above-mentioned adjusted average LNnT values, we then calculated the % difference of the adjusted average LNnT value of a strain relative to the adjusted average LNnT value of strain MP1, which was set to 0%.

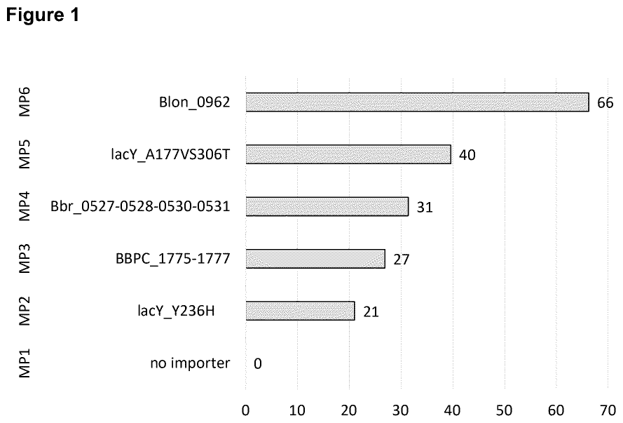

The results of the executed deep well assay experiments, in which the ability of different types of importers to internalize LNT-II (with LNnT formation as a read-out) are shown in FIG. 1. From the data presented in FIG. 1, it can be seen that bacterial cells expressing a recombinant LNnT importer such as an MFS or ABC-type of transporter or even a mutant LacY variant, which is fed with LNT-II, produce LNnT which is seemingly formed due to LNT-II import alone, since any import due to cell lysis or LNT-II import by native E. coli transporters was subtracted during the calculation of the LNnT formation relative to the control. Specifically, the strain expressing the MFS importer Blon_0962 (MP6) produces at least 66% more LNnT than the control strain (MP1). Moreover, the strains MP2 and MP5 expressing LacY mutant variants also show marked increase in LNnT formation compared to the control strain MP1, namely at least 21% and 40%, respectively. Finally, the BBPC_1775-1777 and Bbr_0527-0528-0530-0531 and ABC-transporter systems for sugars is seemingly also able to import LNT-II, with the strains MP3 and MP4 being able to form at least 27% and 31%, respectively, more LNnT than the control strain MP1.

1. A genetically modified cell comprising a recombinant nucleic acid sequence and/or a cluster of recombinant nucleic acid sequences encoding a transporter protein and/or a cluster of proteins capable of importing an acceptor oligosaccharide of at least three monosaccharide units into said cell, wherein said transporter protein and/or cluster of proteins is selected from the group consisting of mutated lactose permease, as shown in table 2, and ABC-importers or MFS importers from a gram-positive bacterium, as shown in table 1.

2. The genetically modified cell according to claim 1, wherein the cluster of transporter proteins capable of importing an acceptor oligosaccharide of at least three monosaccharide units into said cell is an ABC importer selected from the group consisting of:
a. Blon2177, 2176 and 2175 (TP8 in table 1) comprising three sub-units with the amino acid sequences comprising or consisting of SEQ ID NO: 13, 14 and 15 or amino acid sequences with at least 80% identity to SEQ ID: 13, 14 and 15;
b. RHOM_04095, 04100, 04105 (TP9 in table 1) comprising three sub-units with the amino acid sequences comprising or consisting of SEQ ID NO: 35, 36 and 37 or amino acid sequences with at least 80% identity to SEQ ID: 35, 36 and 37;
c. BBPC_1775, 1776, 1777 (TP18 in table 1) comprising three sub-units with the amino acid sequences comprising or consisting of SEQ ID NO: 47, 48 and 49 or amino acid sequences with at least 80% identity to SEQ ID: 47, 48 and 49;
d. Bbr_0527, 0528, 0530, 0531 (TP11 in table 1). comprising four sub-units with the amino acid sequences comprising or consisting of SEQ ID NO: 28, 29, 30 and 50 or amino acid sequences with at least 80% identity to SEQ ID: 28, 29, 30 and 50;
e. HMPREF0373_02960, 0373_02961, 0373_02962 (TP10 in table 1) comprising three sub-units with the amino acid sequences comprising or consisting of SEQ ID NO: 38, 39 and 40 or amino acid sequences with at least 80% identity to SEQ ID: 38, 39 and 40; and
f BBKW_1838, 1839, 1840 (TP17 in table 1) comprising three sub-units with the amino acid sequences comprising or consisting of SEQ ID NO: 44, 45 and 46 or amino acid sequences with at least 80% identity to SEQ ID: 44, 45 and 46 or with the amino acid sequences comprising or consisting of SEQ ID NO: 41, 42 and 43 or amino acid sequences with at least 80% identity to SEQ ID: 41, 42 and 43.

3. The genetically modified cell according to claim 1, wherein the transporter proteins capable of importing an acceptor oligosaccharide of at least three monosaccharide units into said cell is an MFS transporter selected from the group consisting of:
g. Blon:0247 (TP1 in table 1) comprising or consisting of an amino acid sequence of SEQ ID NO: 2 or an amino acid sequence with at least 80% identity to SEQ ID: 2;
h. Blon_0431 (TP2 in table 1 comprising or consisting of an amino acid sequence of SEQ ID NO: 6 or an amino acid sequence with at least 80% identity to SEQ ID: 6;
i. Blon_0788 (TP3 in table 1) comprising or consisting of an amino acid sequence of SEQ ID NO: 7 or an amino acid sequence with at least 80% identity to SEQ ID: 7;
j. Blon_0962 (TP13 in table 1) comprising or consisting of an amino acid sequence of SEQ ID NO: 12 or an amino acid sequence with at least 80% identity to SEQ ID: 12;
k. Blon_2307 (TP14 in table 1) comprising or consisting of an amino acid sequence of SEQ ID NO: 19 or an amino acid sequence with at least 80% identity to SEQ ID: 19; and
l. Blon_2400 (TP4 in table 1) comprising or consisting of an amino acid sequence of SEQ ID NO: 27 or an amino acid sequence with at least 80% identity to SEQ ID: 27.

4. The genetically modified cell according to claim 1, wherein the mutated lactose permease is selected from a lactose permease of SEQ ID NO: 1 or a lactose permease with 90% identity to SEQ ID NO: 1, wherein the lactose permease has one or more mutations selected from the group consisting of Y236N, Y236H, S306T, A177V, H322N, I303F, Y236H+S306T, 177V+Y236H, A177V+1303F, A177V+H322N, A177V+S306T or A177V+Y236N+S306T and wherein the mutation is at the corresponding position in SEQ ID NO: 1.

5. The genetically modified cell according to claim 1, wherein the cell further comprises at least one recombinant nucleic acid sequence encoding at least one glycosyltransferase capable of transferring a glycosyl residue from a glycosyl donor to said acceptor oligosaccharide to synthesize a oligosaccharide product having at least four monosaccharide units.

6. The genetically modified cell according to claim 5, wherein the glycosyltransferase is selected from the group consisting of fucosyltransferases, galactosyltransferases, glucosaminyltransferases, sialic acid transferases, N-acetylglucosaminyl transferases and N-acetylglucosaminyl transferases.

7. The genetically modified cell according to claim 5, wherein genetically modified cell contains one and/or two recombinant nucleic acid sequence encoding one and/or two glycosyltransferases.

8. The genetically modified cell according claim 5, wherein the cell comprise one or more pathways to produce nucleotide-activated sugar selected from the group consisting of glucose-UDP-GlcNac, GDP-fucose, UDP-galactose, UDP-glucose, UDP-N-acetylglucosamine, UDP-N-acetylgalactosamine and CMP-N-acetylneuraminic acid.

9. The genetically modified cell according to claim 1, wherein the cell further comprises a nucleic acid sequence encoding a MFS transporter protein capable of exporting the human milk oligosaccharide product having at least four monosaccharide units into the extracellular medium.

10. The genetically modified cell according to claim 1, wherein the MFS transporter protein capable of exporting the human milk oligosaccharide product having at least four monosaccharide units is Vag.

11. The genetically modified cell according to claim 1, wherein the genetically modified cell does not express a functional lactose importer.

12. The genetically modified cell according to claim 1, wherein said modified cell is selected from the group consisting of Escherichia coli, Corynebacterium glutamicum, Lactococcus lactis, Bacillus subtilis, Streptomyces lividans, Pichia pastoris and Saccharomyces cerevisiae.

13. A method for producing an oligosaccharide having at least four monosaccharide units, said method comprising
m. culturing a genetically modified cell according to claim 1 comprising:
i. a recombinant nucleic acid sequence and/or a cluster of recombinant nucleic acid sequences encoding a transporter protein and/or a cluster of proteins capable of importing an acceptor oligosaccharide of at least three monosaccharide units into said cell, and
ii. at least one recombinant nucleic acid encoding at least one glycosyltransferase capable of transferring a glycosyl residue from a glycosyl donor to said acceptor oligosaccharide to synthesize a oligosaccharide product having at least four monosaccharide units,
wherein the recombinant nucleic acid sequence and/or the cluster of recombinant nucleic acid sequences encoding a transporter protein and/or a cluster of proteins capable of importing said acceptor oligosaccharide of at least three monosaccharide units is selected from the group consisting of mutated lactose permease from table 2 and ABC-importers or MFS importers from a gram-positive bacterium, as shown in table 1,
n. supplying an acceptor oligosaccharide of at least three monosaccharide units to the culture medium,
o. allowing said genetically modified cell to internalize the acceptor oligosaccharide and produce an oligosaccharide having at least four monosaccharide units.

14. (canceled)

15. The method according to claim 13, wherein the acceptor oligosaccharide having at least three monosaccharide units is LNTII and the acceptor oligosaccharide having at least four monosaccharide units is LNT or LNnT.

16. The method according to claim 13, wherein the acceptor oligosaccharide having at least three monosaccharide units is 2′FL or 3FL and the acceptor oligosaccharide having at least five monosaccharide units is LNFP-I.

17. The method according to claim 13, wherein the oligosaccharide having at least four monosaccharide units produced by the method is a human milk oligosaccharide (HMO).

18. The method according to claim 17, wherein the human milk oligosaccharide (HMO) produced by the cell has four monosaccharide units and is selected from the group consisting of LNT, LNnT, DFL and SFL.

19. The method according to claim 17, wherein the human milk oligosaccharide (HMO) produced by the cell has five monosaccharide units and is selected from the group consisting of LNFP-I, LNFP-II, LNFP-III, LNFP-V, LNFP-VI, LST-a, LST-b and LST-c.

20. The method according to claim 17, wherein the human milk oligosaccharide (HMO) produced by the cell has six monosaccharide units and is selected from the group consisting of LNH, LNnH, pLNnH, pLNH-I, DSLNT, LNDFH-I, LNDFH-II and LNDFH-III.

21. The method according to claim 13, wherein the culture medium in which the cultivation is conducted does not contain lactose.
